(19) United States
(12) Patent Application Publication
(10) Pub. No.: US 2025/0001031 A1
(43) Pub. Date: Jan. 2, 2025

(54) PARTICLE COUNTER AND METHOD FOR COUNTING PARTICLES IN A STERILIZATION TUNNEL OF A MEDICAMENT FILLING SYSTEM

(71) Applicant: Hoffmann-La Roche Inc., Little Falls, NJ (US)

(72) Inventors: Mario Zepert, Heiligkreuzsteinach (DE); Patrick Juelly, Brühl (DE)

(21) Appl. No.: 18/292,865

(22) PCT Filed: Jul. 26, 2022
(86) PCT No.: PCT/EP2022/070899

§ 371 Date: Jan. 26, 2024

(30) Foreign Application Priority Data
Jul. 28, 2021 (WO) PCT/EP2021/071122

(51) Int. Cl. A61L 2/28

(52) U.S. Cl. CPC A61L 2/28; A61L 2202/16

(57) ABSTRACT

A particle counter (120) for counting particles in a sterilisation tunnel (110) of a medicament filling system (112) is proposed. The sterilisation tunnel (110) comprises at least one conveyor belt (114). The particle counter (120) comprises:
at least one probe (122) connectable to a particle counter (174) for receiving particles in the sterilisation tunnel (110);
at least one scanner (176) with at least one probe holder (178) for fastening the probe (122), wherein the scanner (176) comprises:
   at least one transverse runner (180) with at least one linear guide (182), wherein the linear guide (182) is configured to guide the probe holder (178) transversely, in particular substantially vertically, with respect to a transport direction (116) of the conveyor belt (114) of the sterilisation tunnel (110);
   at least one bogie (184), wherein the transverse runner (180) is fastened on the bogie (184), wherein the bogie (184) is configured to move the linear guide (182) in the transport direction (116) of the conveyor belt (114); and
   at least one controller (186), in particular a controller (186) connected to the bogie (184), wherein the controller (186) is configured to control a movement of the scanner (176).

Also proposed are a sterilisation tunnel (110) of a medicament filling system (112), a method for counting particles in a sterilisation tunnel (110) of a medicament filling system (112) by means of the particle counter (120), and a computer program and a computer program product.

### TECHNICAL AREA

The invention relates to a particle counting device for particle counting in a sterilisation tunnel of a medicament filling system, a sterilisation tunnel of a medicament filling system, a use of the particle counting device, and a method for particle counting in a sterilisation tunnel of a medicament filling system. The devices and the method according to the present invention can be used, for example, in medical and/or pharmaceutical systems in which special requirements are placed on air purity, for example in clean rooms in which medicament filling systems are located, and in which it is therefore necessary to check the filter systems used to keep the systems clean. Alternatively and/or additionally, however, other possible uses are also conceivable, such as in clean rooms for semiconductor production and/or food production.

### TECHNICAL BACKGROUND

In pharmaceutical plants for the production and/or filling of mediciaments, the production and/or filling of liquid medicaments generally takes place in rooms that are kept sterile, in particular in clean rooms. Filling vessels for liquid medicaments can include, for example, glass injection bottles, vials and/or glass syringes. When filling the medicaments into the vessels, these must generally be free of particles and germs. The cleaning and sterilisation processes can be identical for the different vessels and include, among other things, sterilisation in sterilisation tunnels.

For example, hot air sterilisation tunnels can be used to depyrogenate the vessels before filling them with medicaments. Depyrogenation includes, in particular, sterilisation of the vessels with dry heat at 160 to 400° C. In general, a virtually particle-free laminar airflow is required. The particle-free nature of the airflow can be achieved using filters installed in hot air sterilisation tunnels, for example HEPA filters. The quality of the installed filters can be checked at regular intervals using so-called leak penetration tests, in order to be able to record any excess particle quantities in the airflow. For example, a filter surface can be scanned in a meandering manner using a measuring probe.

Such leak penetration tests can, for example, be part of regular maintenance of the sterilisation tunnels as part of “standard operating procedures” (SOP) and include so-called DEHS tests, where DEHS (di-ethyl-hexyl-sebacate) denotes the aerosol used for testing. In a leak penetration test, an aerosol is generally applied in front of the filter to be examined and a particle concentration is determined in front of the filter, in particular on the side of the untreated air. On the opposite side of the filter, especially on a clean air side, the filter surface can then be searched for any increased particle passages using a probe. In general, the probe is guided manually in the leak penetration test. The funnel-shaped probe can be inserted into the sterilisation tunnel on an extendable pipeline. With the help of the pipeline, an inspector can manually guide the probe along a conveyor belt under the air outlet of the filter in a predetermined time, with a predetermined path and a predetermined speed. The testing process and the associated parameters are generally defined in SOPs.

Via the funnel-shaped probe and the pipeline, the air flowing out of the filters can be fed by a particle counter, which can determine the concentration of the particles contained in the air. If a particle concentration is found to be above a threshold specified in the SOP, it can be assumed that the filter is permeable and/or leaking. If the particle concentration is determined again at this position, the filter can be classified as functional if the particle concentration is not increased and the filter can be classified as non-functional in the case of an increased particle concentration. Non-functional filters generally require complex replacement of the filters.

However, known devices and methods for carrying out leak penetration tests have numerous technical challenges. In particular, current manual measurement methods generally have poor reproducibility because the results of the manual methodology depend significantly on the inspector carrying out the test. For example, difficulties arise with regard to maintaining specified test speeds. In addition, depending on the depth of the sterilisation tunnel, a longer tube length may be required for the measuring probe. For example, in some sterilisation tunnels, filter surfaces have to be examined at a distance of up to 5 m, which represents a particular challenge with a hand-held probe. In addition, when determining a particle concentration increased at certain points, it can be a challenge to determine the exact position and find it again. This can, in particular, involve increased time expenditure. More time-consuming examinations of the filter system in the filling systems generally also result in less availability of the filling systems for the actual filling of the medicaments.

In Patrick Jülly's bachelor's thesis “Concept development for a semi-automatic scanning robot for particle counting in the sterilisation tunnel” in the Department of Economics and Technology Management at the Wilhelm Büchner University in Darmstadt, a scanning robot for particle counting in the sterilisation tunnel is described.

### OBJECT OF THE INVENTION

It would therefore be desirable to provide a particle counting device for particle counting in a sterilisation tunnel of a medicament filling system, a sterilisation tunnel of a medicament filling system, a use of the particle counting device, and a method for particle counting in a sterilisation tunnel of a medicament filling system, which at least largely avoid the disadvantages of known devices, uses and methods. In particular, a reproducible examination of filters in sterilisation tunnels should be made possible, which is also economical in terms of time and costs.

### General Description of the Invention

The problem is addressed by a particle counting device for particle counting in a sterilisation tunnel of a medicament filling system, a sterilisation tunnel of a medicament filling system, a use of the particle counting device, and a method for particle counting in a sterilisation tunnel of a medicament filling system with the features of the independent patent claims. Advantageous developments, which can be implemented individually or in any combination, are presented in the dependent claims.

Below, the terms “have,” “consist of,” “comprise,” or “include,” or any grammatical variations thereof, are used non-exclusively. Accordingly, these terms can refer both to situations in which, in addition to the features introduced by these terms, no further features are present, or to situations in which one or more further features are present. For example, the expression “A has B”, “A consists of B”, “A comprises B” or “A includes B” can refer to the situation in which, apart from B, no other element is present in A (i.e. to a situation in which A consists exclusively of B), as well as to the situation in which, in addition to B, one or more other elements are present in A, for example element C, elements C and D or even further elements.

Furthermore, it should be noted that the terms “at least one” and “one or more” as well as grammatical variations of these terms, when used in connection with one or more elements or features and intended to express that the element or feature can be provided singly or in multiples, are generally used only once, for example when the feature or element is introduced for the first time. If the feature or element is subsequently mentioned again, the corresponding term “at least one” or “one or more” is generally no longer used, without limiting the possibility that the feature or element can be provided singly or in multiples.

Furthermore, the terms “preferably”, “in particular”, “for example” or similar terms are used below in connection with optional features, without this limiting alternative embodiments. Thus, features introduced by these terms are optional features and these features are not intended to limit the scope of protection of the claims and, in particular, the independent claims. Thus, as those skilled in the art will recognise, the invention can also be implemented using other embodiments. Similarly, features introduced with “in an embodiment of the invention” or with “in an exemplary embodiment of the invention” are understood to be optional features, without this being intended to limit alternative embodiments or the scope of protection of the independent claims. Furthermore, these introductory expressions should not affect any possibilities of combining the features introduced by them with other features, whether they be optional or non-optional features.

In a first aspect of the present invention, a particle counting device for counting particles in a sterilisation tunnel of a medicament filling system is proposed. The sterilisation tunnel comprises at least one conveyor belt. The particle counting device comprises at least one probe, which can be connected to a particle counter, for receiving particles in the sterilisation tunnel. Furthermore, the particle counting device comprises at least one scanner with at least one probe holder for mounting the probe. The scanner includes at least one transverse runner with at least one linear guide. The linear guide is configured to guide the probe holder transversely, in particular essentially perpendicularly, to a transport direction of the conveyor belt of the sterilisation tunnel. Furthermore, the scanner includes at least one bogie. The transverse runner is mounted on the bogie. The bogie is configured to move the linear guide in the transport direction of the conveyor belt. Furthermore, the scanner comprises at least one controller, in particular a controller connected to the bogie, wherein the controller is configured to control a movement of the scanner.

The bogie can be configured to move itself and the transverse runner, in particular the transverse runner with the probe, in a two-dimensional space. The particle counting device can, in particular, in each case comprise a drive for guiding the probe holder transversely, in particular essentially perpendicularly, to the transport direction of the conveyor belt of the sterilisation tunnel and for moving the bogie along the transport direction of the conveyor belt. Both drives can be moved respectively with the help of a motor, as is explained in more detail below. In particular, the particle counting device can be designed such that the movement of the bogie is independent of guiding the probe holder.

The term “medicament filling system” as used herein is a broad term which should be given its ordinary and current meaning as understood by those skilled in the art. The term is not limited to a specific or adapted meaning. The term can, without limitation, refer in particular to any system that is configured to fill one or more medicaments into vessels. The vessels can be, for example, vials, in particular injection bottles, or syringes, in particular glass syringes. The medicament filling system can in particular be set up and operated in a clean room. The medicament filling system can in particular include the sterilisation tunnel, which is described in more detail below. The sterilisation tunnel can be configured to ensure that the vessels are free of particles and/or germs. Furthermore, the medicament filling system can include at least one washer, at least one filling system and/or at least one inspection machine. The washer can be configured to clean the vessels with water for injection (WFI). The medicament filling system can be configured to transport the vessels from the washer to the conveyor belt of the sterilisation tunnel. Optionally, the medicament filling system can include at least one freeze-drying system. The freeze-drying system can be configured to freeze-dry filled medicaments after filling, in particular to ensure a shelf life of the filled medicaments.

The term “sterilisation tunnel” as used herein is a broad term which should be given its ordinary and common meaning as understood by those skilled in the art. The term is not limited to a specific or adapted meaning. The term can, without limitation, refer in particular to a device which is configured to free one or more objects that pass through the sterilisation tunnel, for example medicament containers, from microscopic contamination or to at least partially eliminate such contamination. The sterilisation tunnel can, in particular, be configured to sterilise the objects. Sterilisation can in particular refer to a process in which the objects are completely or partially freed from adhering germs and/or in which a reduction in germs occurs on and/or in the objects. This germ reduction can take place, for example, through heat treatment and/or through chemical treatment of the objects, for example through treatment with a disinfectant gas and/or with superheated steam. Germ reduction preferably takes place by heat treatment in the sterilisation tunnel.

The term “conveyor belt” as used herein is a broad term which should be given its ordinary and common meaning as understood by those skilled in the art. The term is not limited to a specific or adapted meaning. The term can, without limitation, refer in particular to a device which is configured to transport at least one other device or at least one other element, for example one or more vessels to be filled, and/or to drive a movement of the other device or other element. The conveyor belt can in particular comprise at least one drive element, for example at least one drive element running in a circuit through the sterilisation tunnel. In particular, the conveyor belt can be at least partially permeable to air, for example in such a way that at least part of the supply air supplied to the conveyor belt can pass through the conveyor belt. For example, the conveyor belt includes a wire mesh. The wire mesh can in particular ensure the necessary air permeability, flexibility and heat resistance. The transport can, for example, take place continuously or discontinuously or in cycles, so that, for example, continuously operating sterilisation tunnels or cycled sterilisation tunnels can be used. The transport direction can, for example, be a main direction of movement of the vessels to be filled within the sterilisation tunnel. The transport direction can be fixed or can also change, for example locally or over time. For example, the transport direction can be directed from an inlet of the sterilisation tunnel towards an outlet. The transport direction can, for example, be a main direction of movement of the vessels within the sterilisation tunnel. The transport direction can be fixed or can also change, for example locally or over time. For example, the transport direction can be directed from an entrance of the sterilisation tunnel towards an exit of the sterilisation tunnel. The transport direction can therefore also be referred to as the conveying direction.

The term “particulate filter” as used herein is a broad term that should be given its ordinary and common meaning as understood by those skilled in the art. The term is not limited to a specific or adapted meaning. The term can, without limitation, refer in particular to a device which is configured to at least partially separate suspended matter, alternatively also referred to as “particles”, from at least one medium, in particular from at least one gaseous medium such as air, which flows through the particulate filter. The at least partial separation can in particular involve a complete removal of the suspended matter from the medium flowing through or, alternatively, a reduction in a concentration of the suspended matter in the medium flowing through, for example by at least 85%, preferably by at least 95%, particularly preferably by at least 99.95%, of a concentration of suspended matter with a particle size in the range of 0.1 μm to 0.3 μm. The particulate filter can, in particular, separate one or more suspended materials from the medium flowing through the particulate filter, for example bacteria, viruses, pollen, dust, aerosols and/or smoke particles. The particulate filter can comprise at least one filter selected from the group consisting of: an EPA (Efficient Particulate Air) filter; an HEPA (High Efficiency Particulate Air) filter; a ULPA (Ultra Low Penetration Air) filter. Particularly preferably, the particulate filter can comprise at least one HEPA filter.

The term “particle counting device” as used herein is a broad term which should be given its ordinary and common meaning as understood by those skilled in the art. The term is not limited to a specific or adapted meaning. The term can, without limitation, refer in particular to a device which enables particle counting in a sterilisation tunnel of a medicament filling system. The particle counting device can, in particular, be used for a particle counting process. During the particle counting process, the particle counting device can, in particular, have the task of guiding a probe in the sterilisation tunnel. The particle counting device can be configured for automated, in particular partially or fully automated, guidance of the probe in the sterilisation tunnel. Alternatively and/or additionally, the particle counting device can be configured to supply to a particle counter a gaseous medium, for example air, with the particles contained therein and to be counted.

The particle counting device can be configured to use the probe to scan areas of different sizes below a particulate filter of the sterilisation tunnel on a predetermined path at a predetermined speed. The probe can be configured to absorb the air flowing through the particulate filter and to direct it to the particle counter connected to the probe. The particle counter can, as is explained in more detail below, be configured to count and/or measure the particles present in the filtered air.

The particle counting device can further comprise the at least one particle counter which can be connected to the probe. The term “particle counter” as used herein is a broad term which should be given its ordinary and common meaning as understood by those skilled in the art. The term is not limited to a specific or adapted meaning. The term can, without limitation, refer in particular to a device which is configured for the quantitative and/or qualitative recording of particles in a gaseous medium, in particular in air. The particle counter can in particular refer to a device which is configured for counting particles in the gaseous medium. The particle counter can be configured to optically record the particles in the gaseous medium. For example, the particle counter can comprise at least one light source, at least one measuring cell which contains at least part of the gaseous medium, and at least one photodetector which can detect the light emitted by the light source and scattered and/or diffracted by the particles contained in the gaseous medium. Based on the signal detected by the photodetector, the particles contained in the gaseous medium can be recorded qualitatively and/or quantitatively. The gaseous medium can flow through the measuring cell of the particle counter continuously or, alternatively, the measuring cell can be filled discontinuously with the gaseous medium to be examined. The particle counter can therefore continuously or discontinuously record the particles in the gaseous medium. The particle counter can, in particular, record a number, a size and/or a concentration of the particles in the gaseous medium. The particle counter can record particles with a size in the range from 10 nm to 1000 μm, preferably in the range from 100 nm to 100 μm, particularly preferably in the range from 0.3 μm to 10 μm. Accordingly, the term “particle counting”, alternatively also referred to as the “particle count”, can in principle refer to any process for the quantitative and/or qualitative recording of particles in a gaseous medium, particularly in air.

In particular, the particle counter can be designed as a stationary particle counter and the particle counter and the probe can be connected to one another by means of at least one pipeline, in particular a flexible pipeline. The pipeline can be part of the particle counter and/or part of the particle counting device. The probe and the pipeline can be configured to suck in air and supply it to the particle counter. The particle counter can, in particular, be configured to count and/or measure particles. If a measured particle count and/or a measured particle concentration exceeds a defined value, it can be concluded that there is permeability, in particular a leak, in the particulate filter. Measurements can then be carried out more precisely at the position of the particle counting device on the conveyor belt with the measured increased particle concentration or particle count. If a new measurement does not reveal an increased particle concentration or particle count, the particulate filter can be classified as functional. However, if an increased particle concentration or particle count is repeatedly measured, the particulate filter must be replaced, which is a complex process.

The term “probe” as used herein is a broad term which should be given its ordinary and common meaning as understood by those skilled in the art. The term is not limited to a specific or adapted meaning. The term can, without limitation, refer in particular to a device that is configured to transmit information and/or objects. In particular, the probe can be configured for object transmission. For example, the probe can be configured to receive particles at a first position and to deliver them to a second position that is different from the first position. Thus, in particular, the probe can make it possible to count particles at the first position while the particles are recorded at the second position. Alternatively and/or additionally, direct recording of the particles at the first position and information transmission of a particle count result to the second position may also be possible. The probe can, in particular, be an isokinetic probe. The term “isokinetic probe” substantially refers to any probe that is configured to receive a sample, in particular particles, from flowing fluids. In particular, the fluid flowing into the isokinetic probe can have a velocity which corresponds to a velocity of a fluid in the immediate vicinity of the isokinetic probe. As a result, falsification of a particle count of the fluid flowing into the isokinetic probe can be avoided or at least reduced during particle uptake.

The term “scanner” as used herein is a broad term that should be given its ordinary and common meaning as understood by those skilled in the art. The term is not limited to a specific or adapted meaning. The term can, without limitation, refer in particular to any device which is configured to scan an area, in particular a two-dimensional area, in a systematic and/or regular manner. In particular, the area can be an area within the sterilisation tunnel, in particular an area below the at least one particulate filter, in particular below at least one filter surface of the particulate filter, of the sterilisation tunnel. The scanner can preferably be configured to scan the area below the at least one particulate filter in at least partially overlapping paths, as is explained in more detail below. Furthermore, the scanner can preferably be configured to scan a travel path with a meandering pattern by alternating movements of the probe transversely and parallel to the transport direction, as is explained in more detail below.

As stated above, the scanner includes the at least one probe holder. The probe holder can be configured for mounting the probe. The term “probe holder” as used herein is a broad term that should be given its ordinary and common meaning as understood by those skilled in the art. The term is not limited to a specific or adapted meaning. The term can, without limitation, refer in particular to any device that is configured to mount a probe of any design to a component of the scanner. In particular, the probe holder can be configured to fasten the probe on a guide carriage of the linear guide. The probe holder itself can be mounted on the component of the scanner, in particular on the guide carriage. The probe holder can therefore have at least one recess in which the probe holder can be at least partially received. Furthermore, the recess can be configured to at least partially receive the pipeline. In particular, the probe holder can have at least one groove for receiving the probe. Furthermore, the probe holder can have at least one clamping plate, which is configured to fix the probe. Other configurations are certainly also conceivable. Furthermore, the probe holder can be made at least partly of polyoxymethylene (POM). This can result in a reduced weight of the particle counting device. Other materials are certainly also conceivable.

As stated above, the scanner includes at least one transverse runner with at least one linear guide. The term “transverse runner” as used herein is a broad term that should be given its ordinary and common meaning as understood by those skilled in the art. The term is not limited to a specific or adapted meaning. The term can, without limitation, refer in particular to a component of the particle counting device which is configured to guide at least one component of the particle counting device transversely, in particular essentially perpendicularly, to the transport direction of the conveyor belt. The transverse runner can therefore also be referred to as a transverse axis. The transverse runner has at least one linear guide, which is explained in more detail below.

The term “linear guide” as used herein is a broad term that should be given its ordinary and common meaning as understood by those skilled in the art. The term is not limited to a specific or adapted meaning. The term can, without limitation, refer in particular to any device which is configured to enable a rectilinear, guided movement of a component from one point to another point. The linear guide can in particular be configured to move the probe transversely to the transport direction of the sterilisation tunnel in a straight line and at a constant speed. The linear guide can be configured to limit six degrees of freedom of the component, in particular three translational degrees of freedom and three rotational degrees of freedom, to one, in particular single, translational degree of freedom of the component. The linear guide can comprise at least one guide rail, in particular a profiled guide rail, in particular a T-shaped guide rail, or a round shaft. Furthermore, the linear guide can comprise at least one guide carriage, in particular at least one guide carriage mounted on the guide rail or the round shaft. The probe can be mountable or mounted on the guide carriage, in particular by means of the probe holder. The linear guide can therefore be configured to guide the probe. The linear guide can in particular have at least one slide bearing, in particular for the guide carriage. The slide bearing can be designed to be lubricant-free. However, other bearings such as ball or roller bearings are certainly also conceivable.

The T-shaped guide rail makes it substantially possible for the guide carriage with floating bearing to be designed in the direction transverse, in particular perpendicular, to the transport direction of the conveyor belt and/or in the transport direction of the conveyor belt. The floating bearing allows the guide carriage to have some play in the selected direction. This makes it possible to compensate for manufacturing tolerances in the design. Without floating bearings, the system might be rigid, which would mean the guide carriage might tilt, for example. In particular, the guide rail can be configured as a floating bearing, in particular as a floating bearing in a direction transverse, in particular perpendicular, to the transport direction of the conveyor belt. This makes it possible for the guide carriage to compensate for small differences in height, in particular in an overall system. A length of the guide carriage can correspond to a flange width of a motor.

The linear guide can in particular be a Drylin® T miniature linear guide (Igus, Germany) made of hard anodised aluminium. The Drylin® T miniature linear guide can have a total height of 16 mm, a guide carriage length of 42 mm, a guide carriage width of 32 mm and a rail length that can be individually adjusted. The Drylin® T miniature linear guide can therefore have a low overall height and slide bearings. A length of the guide carriage may be less than or equal to a flange size of a stepper motor. The Drylin® T miniature linear guide can be maintenance-free and lubricant-free. The Drylin® T miniature linear guide can have polymeric high-performance sliding elements with good wear and friction properties. Furthermore, the Drylin® T miniature linear guide can have a T-shaped guide rail. Contamination by oils and fats is strictly avoided because no lubricant is necessary. Dirt or dust particles strictly cannot adhere. According to the manufacturer, this system is insensitive to water, chemicals, heat and shock. Requirements in terms of freedom from lubricants and cleanability can therefore be met. The length of the guide rails can be individually adapted to different tunnel widths of the sterilisation tunnel.

The linear guide can be configured to guide the probe transversely to the transport direction of the sterilisation tunnel. The guide carriage of the linear guide can, in particular, be configured for mounting all components that are necessary for generating and carrying out the linear movement, and the probe holder, as is explained in more detail below. In particular, the probe holder can be mounted on the guide carriage.

The linear guide, in particular the guide rail, can be mounted on a base plate, in particular on a base plate made of aluminium, in particular by means of at least one screw connection. The base plate can be designed to be interchangeable. If the guide rail shows wear, it can be replaced at any time if necessary. The linear guide can be mounted on the bogie using the base plate. In particular, the base plate can be mounted on the bogie by means of at least one connection selected from the group consisting of: at least one screw connection, at least one click connection, at least one tension lever connection. The screw connection can, in particular, include knurled screws and/or cylinder head screws. In particular, the base plate can comprise a plurality of boreholes, particularly in order to mount the transverse runner on the bogie. Due to the high number of transverse runners that need to be changed every year, around six times per year, it can be advantageous to mount the transverse runner on the bogie with knurled screws. This makes it by all means possible to create a compact construction. This means that a tool-free change is certainly possible and the transverse runner can be firmly but releasably connected to the bogie. The base plate can be adjusted, particularly in order to save weight, by notching out material that is not required. Further embodiments for mounting the transverse runner to the bogie are certainly also conceivable, such as click systems or tension levers.

The linear guide can have at least one drive, in particular a linear drive. The drive can be configured to move the guide carriage on the guide rail. The drive can be configured to scan an entire conveyor belt width of the conveyor belt of the sterilisation tunnel. The drive may be selected from the group consisting of: a spindle drive; a toothed belt drive, a rack and pinion drive. Other embodiments are certainly also conceivable.

The toothed belt drive can in particular have at least one toothed belt and at least two toothed belt wheels. One of the toothed belt wheels can be configured to be driven by a motor. The toothed belt can be configured for guiding over the toothed belt wheels. An object mounted on the toothed belt, for example a carriage, can be moved in this way. The toothed belt can have a plurality of teeth. Furthermore, the toothed belt wheels can each have a plurality of teeth. A shape of the teeth of the toothed belt can be adapted to a shape of the teeth of the toothed belt wheel. This results in a positive power transmission. With the right tooth shape, backlash-free drive can substantially be achieved. Changing the direction of rotation of the toothed belt wheels can achieve linear movement of the toothed belt, and thus the carriage, in both directions. The running of a toothed belt drive is generally quiet. A slip-free and synchronous movement can be substantially transmitted in a shock-absorbing manner and with a low preload. A travel speed of the guide carriage can be controlled via a rotational speed of a motor. The toothed belt drive structure can be very small in height and is certainly suitable for quick positioning of smaller loads. Lubrication is not at all necessary. Toothed belt drives are certainly already available on the market as a complete unit. This can certainly simplify a construction.

The spindle drive can have at least one spindle. Furthermore, the guide carriage and the guide rail can be components of the spindle drive. The guide carriage can, in particular, have an internal thread that is compatible with a thread of the spindle. The spindle can be configured to be driven by a motor and a rotation of the spindle can be converted into a linear movement of the guide carriage by interlocking of the threads. The guide rail can prevent the guide carriage from rotating about an axis of the spindle. The spindle can, in particular, be a ball screw or a trapezoidal spindle. A direction of movement of the guide carriage can be controlled by a direction of rotation of the motor. A pitch of the thread can indicate an advance of the guide carriage per spindle revolution. A greater pitch can result in a greater travel speed per revolution. The spindle drive can certainly generate considerably louder running noises than the toothed belt drive and may certainly require lubrication.

Preferably, the linear guide can have at least one rack and pinion drive. The rack and pinion drive can have at least one rack and at least one spur gear. With a rack and pinion drive, a rotating movement of the spur gear is converted into a linear movement. There are, in principle, two options here. On the one hand, the spur gear can be designed to be stationary with a motor as a drive unit and configured to drive a linearly mounted rack. The linearly mounted rack can be configured to move depending on the direction of rotation of the gear. On the other hand, the rack can be fixed and configured to move the spur gear and a drive unit linearly, in particular along the rack. In particular, the drive unit with the spur gear can be mounted on the guide carriage of the linear guide. This flexibility is certainly a major advantage of rack and pinion drives. An entire length of the rack can certainly also be used as a travel path. Due to the functionality described, the rack and pinion drive can certainly be constructed compactly and, if the material is selected appropriately, it can generally be operated without lubrication. The rack and pinion drive is substantially positive, slip-free, and can achieve a high degree of efficiency if manufactured precisely. Like the toothed belt drive, the rack and pinion drive is generally quiet.

The rack can, in particular, have a round cross-section. The rack can, in particular, be configured to provide additional guidance for the probe holder. The round cross-section can certainly simplify production for the additional guidance. The rack can certainly be shortened to required lengths. The rack can, in particular, be made of an austenitic stainless steel. In particular, the rack can be made of an austenitic stainless steel with a diameter of 10 mm, a delivery length of 1000 mm, a weight of 560 g and a modulus of elasticity E of 200,000 N/mm² and a module m of 1, wherein the module m corresponds to a toothing dimension for gears. The material has the material number 1.4305 according to EN 10027-2:1992-09.

As stated above, depending on the tunnel type, the rack can be shortened to the required length. Starting from the largest required length of the transverse runner, a deflection f of the rack can be calculated. This makes it possible to check how the rack deflects under its own weight. The knowledge gained in this way can be taken into account when designing the transverse runner. A tunnel type of sterilisation tunnel with a conveyor belt width of 800 mm can be used for the calculation. The greatest possible deflection of the rack is certainly to be expected here. To calculate the deflection f, the following formula can be used:

$$f = \frac{F * {l}^{3}}{384 * E * I} \qquad ( 1 )$$

Thus, F corresponds to a weight of the rack, l to a length of a distributed load and/to a second moment of area.

The rack can be clamped on two sides on two rack holders, each 10 mm wide. The following length of the distributed load/can thus result:

$$l = 800 mm - 2 * 10 mm = 780 mm \qquad ( 2 )$$

A weight m of the shortened rack can be determined in an additional calculation:

$$m = \frac{8560 g * 780 mm}{1000 mm} = 436.8 g = 0.4368 kg \qquad ( 3 )$$

The weight force F of the rack can be calculated as follows:

$$F = m * g = 0.4 \; 368 kg * 9.81 \frac{m}{{s}^{2}} = 4.287 N \qquad ( 4 )$$

The second moment of area I (TBB) can be determined as follows:

$$I = \frac{π * {d}^{4}}{64} \frac{π * {( 10 mm )}^{4}}{64} = 490.87 {mm}^{4} \qquad ( 5 )$$

The determined values can be used in formula (1) to determine the deflection f of the rack:

$$f = \frac{4.287 N * {( 780 mm )}^{3}}{382 * 200000 \frac{N}{{mm}^{2}} * 4 90.87 {mm}^{4}} = 0.0 \; 54 mm \qquad ( 6 )$$

The determined value of deflection f is 0.054 mm at the longest length. The guide carriage with the floating bearing in the direction transverse to the transport direction of the conveyor belt can, in principle, easily compensate for this difference. The deflection of the rack due to its own weight is generally so small that it generally does not need to be taken into account when designing the transverse runner. With shorter lengths of the transverse runner, it can generally be assumed that the rack will deflect less.

The transverse runner can also have at least one, preferably at least two, rack mounts. The rack mount can be configured to fix the rack, particularly on the base plate of the transverse runner. The rack mount can, in particular, be made of aluminium. Other embodiments are certainly also conceivable. The rack mount can, in particular, have an upper part and a lower part. The lower part can be configured to be fixed on the base plate, in particular by means of at least one screw connection. In particular, as stated above, the transverse runner can have two of the rack mounts and the lower parts can each be arranged at ends of the guide rail. The upper part can be configured to be screwed to the lower part and can further be configured to fix the rack, particularly in such a way that rotation and/or displacement of the rack is avoided or at least reduced.

As stated above, the rack and pinion drive can in particular be configured to move the guide carriage over the rack, which can in particular be fixed on the base plate of the transverse runner, by means of a rotary movement of the spur gear. The spur gear can in particular be made of polyoxymethylene (POM). Compared to a spur gear made of metal, running noise can generally be minimised and lubrication can certainly be dispensed with. In addition, the spur gear made of polyoxymethylene (POM) can certainly have a comparatively low weight and comparatively low manufacturing costs.

The spur gear can, in particular, have 19 teeth. Furthermore, the spur gear can have a module m of 1, wherein the module m corresponds to a toothing dimension for gears. This results in the following values for a pitch diameter d_{z} and a distance travelled l_{z} for a full revolution of the gear:

$${d}_{z} = m * z \qquad ( 7 )$$

$${d}_{z} = 1 * 19 = 19 mm \qquad ( 8 )$$

$${l}_{z} = π * {d}_{z} \qquad ( 9 )$$

$${l}_{z} = π * 19 mm = 56.69 mm \qquad ( 10 )$$

Thus, a motor connected to the rack and pinion drive can perform one revolution per second to maintain a required particle counting speed of 5.9 cm/s. This value can be used to program the motor.

The spur gear can be clamped as a non-positive connection with a set screw with a cutting ring on a motor shaft of the motor of the rack and pinion drive. The motor shaft can have a flattening. The flattening on the motor shaft can offer the set screw a comparatively larger area in order to generate a comparatively higher contact pressure. When tightening the set screw, a burr may appear on a surface of the motor shaft. Flattening of the motor shaft means that dismantling the spur gear is, in principle, not hindered by the created burr. Slippage of the motor shaft is also generally hindered.

By precisely positioning the spur gear relative to the rack, the teeth of the spur gear can, in principle, mesh with the teeth of the rack in the best possible way. This generally reduces any play between interlocking teeth, at least as far as possible. An optimal centre distance a of rack and gear is calculated as follows, wherein do corresponds to a partial circle line and d corresponds to a diameter of the rack:

$${d}_{0} = \frac{d}{2} * m \qquad ( 11 )$$

$${d}_{0} = \frac{10 mm}{2} * 1 = 5 mm \qquad ( 12 )$$

$$a = {d}_{0} + \frac{{d}_{z}}{2} \qquad ( 13 )$$

$$a = 5 mm + \frac{19 mm}{2} = 14.5 mm \qquad ( 14 )$$

A mounting bracket, in particular made of aluminium, can be mounted, in particular screwed, to threaded holes in the guide carriage. The stepper motor can also be mounted, in particular screwed, on the mounting bracket and can be configured to position the spur gear with the centre distance a from the rack. The rack and pinion drive thereby functions largely correctly. By rotating the motor shaft and thus the spur gear, the guide carriage can move on the guide rail. Boreholes can be provided on the mounting bracket for mounting the probe holder.

By using a rack and pinion drive with the properties shown above, with skillful selection and arrangement of the components, the guide carriage with the probe can, in principle, be moved over almost the entire length of the rack, and thus an entire conveyor belt width. The rack and pinion drive is generally particularly suitable here because the rack length can be set variably, regardless of most components. With little effort and by adjusting the length of a few components, such as the rack, suitable transverse runners can generally be designed for any type of tunnel.

The drive may include at least a first motor, wherein the drive includes at least a first motor selected from the group consisting of: a servo motor; a stepper motor. Other types of motors are certainly also conceivable. The terms “first motor” and “second motor” are to be viewed as pure descriptions, without specifying a sequence or ranking and, for example, without excluding the possibility that a plurality of types of first motors or second motors or exactly one type can be provided. Furthermore, additional motors, for example one or more third motors, may be present.

The term “motor” as used herein is a broad term that should be given its ordinary and common meaning as understood by those skilled in the art. The term is not limited to a specific or adapted meaning. The term can, without limitation, refer in particular to any engine that is configured to perform mechanical work, in particular by converting a form of energy, such as thermal, chemical, hydraulic, pneumatic or electrical energy, into kinetic energy.

The servo motor can in particular be a synchronous servo motor. The servo motor can, together with a servo regulator, form a servo drive. The synchronous servo motor can, in particular, include a stator with copper wire windings and a rotor with permanent magnets. The permanent magnets can be configured to form a constant magnetic field around the rotor. The servo regulator can be configured to supply the stator with alternating current, which creates a second magnetic field, in particular a rotating field. The rotating field can exert a force on the magnetic field of the rotor, which rotates synchronously with the rotating field. Through a change in the current frequency, the speed of the rotating field and thus the rotor can change. The magnitude of the current can be used to determine the magnitude of the electromagnetic force and thus the rotor torque. A higher-level control unit can pass on a speed and a target position of the motor to the servo motor via the servo regulator. The motor can be configured to return current actual values, speeds and positions to the servo regulator. If there are deviations, the speed and current can be readjusted via the control unit. Servo motors generally have high dynamics, high positioning accuracy and a high overload capacity within a wide speed range. Further features of the servo motor include high speed accuracy, a short acceleration time, a short torque response time, a high stall torque and a small mass moment of inertia. In addition, they generally have a compact design and are certainly low in weight in relation to their performance.

The stepper motor can, in particular, be selected from the group consisting of: a permanently excited stepper motor, a reluctance stepper motor; a hybrid stepper motor. The stepper motor can preferably be a hybrid stepper motor. The hybrid stepper motor can, in principle, combine the advantages of the permanently excited stepper motor and the reluctance stepper motor. The hybrid stepper motor can, in particular, be configured to achieve very small step angles. The stepper motor, particularly the hybrid stepper motor, can comprise at least one rotor, in particular at least one permanent magnet, in particular at least one cylindrical permanent magnet with axial polar alignment. Furthermore, the stepper motor, in particular the hybrid stepper motor, can have at least one stator field, in particular a fixed stator field, with a plurality of stator coils, for example eight stator coils. The stepper motor, in particular the hybrid stepper motor, can be configured to rotate the rotor by a defined angle or by a defined step by alternately controlled stator coils.

At least two rotor shells with a large number of teeth, for example 50 teeth each, which are twisted against each other by one tooth width, can be arranged around the permanent magnet, one behind the other and firmly connected to the rotor. The rotor shells can be configured to accept magnetisation of the permanent magnet. The stator field can also have a large number of teeth, for example 48 teeth. The stator coils of the stator field can be energised one after the other, offset by 45 degrees per step, which creates a changing electromagnetic field. The magnetised rotor shells can align their teeth with the electromagnetic field of the stator coils. Due to the different number of teeth in the stator field and the rotor shells, the rotor rotates, for example by 1.8° per step.

The first motor can, in particular, comprise a first motor driver. The terms “first motor driver” and “second motor driver” are to be viewed as pure descriptions, without specifying a sequence or ranking and, for example, without excluding the possibility that a plurality of types of first motor drivers or second motor drivers or exactly one type can be provided. Furthermore, additional motor drivers, for example one or more third motor drivers, may be present. The first motor driver can be configured to transmit signals, in particular signals from the controller, to the first motor and/or to supply the first motor with a voltage. For this purpose, the first motor driver can be connected to a mains adaptor, for example. The mains adaptor can be configured to convert a voltage to a voltage required by the first motor.

In particular, the drive of the linear guide can include at least one NEMA 17 stepper motor (Stepperonline, China) and a related stepper motor driver. The NEMA 17 stepper motor can have a manufacturer part number 17HS24-2104S, a holding torque M1 of 0.65 Nm, a step angle of 1.8°, a flange dimension of 42 mm, a length of 60 mm and a weight of 500 g. The NEMA 17 stepper motor is generally low in weight for its size and has a large selection of different holding torques. If the selected holding torque is not sufficient to move the guide carriage with the probe holder and the probe, it is certainly possible to replace the stepper motor with a more powerful stepper motor without having to change anything in the design of the transverse runner. Despite its small size, the Nema17 stepper motor can also be suitable for generating the power required to drive the linear guide of the transverse runner. A stepper motor with a smaller holding torque can certainly also be used. This can certainly mean a weight saving for the transverse runner. With a flange dimension of 42 mm, NEMA 17 stepper motors are generally only slightly wider than a diameter of the probe, which can be 36 mm, for example. This means that the probe is generally only minimally restricted by the motor when reaching external positions, for example by 3 mm in each case. The motor can generally also specify a maximum length of the guide carriage based on its flange dimension. The drive shaft of the motor can have a milled flattening. The flattening can, in principle, make it easier to mount the spur gear on the motor shaft by means of a force-fit shaft-hub connection. Furthermore, the second motor can be a stepper motor of the OMC Stepperonline 17HS24-2104S type with a step generator of the OMC Stepperonline DM556N type.

Hybrid stepper motors generally have a high holding torque even when stationary, without overheating, and are generally small in size. For simple positioning tasks with a hybrid stepper motor, no distance measuring system is generally required because the steps can be counted. However, there is generally no monitoring of the steps and therefore there is generally no position feedback. This open control circuit is also known as an open loop system. This has the basic disadvantage that steps can be skipped in the event of external interference or overload, for example. For the travel path of the probe, this would certainly mean that the accuracy of the travel path would decrease. One possibility for position feedback is to use a stepper motor with an encoder. The encoder can be configured to count and monitor the number of steps. With such a closed control circuit, also known as a closed loop system, a suitable output stage is generally also connected between the controller and the encoder, which processes the information coming from the encoder and transmits it to the controller. This means that the controller can certainly determine, save and adjust, if necessary, the current position. However, an encoder generally increases the overall length and weight of the stepper motor. Hybrid stepper motors can, in principle, only generate full torque up to a certain speed. If the speed is increased, the torque generally decreases above a certain speed. The torque drop can be taken from a characteristic curve of the motor. If the torque is exceeded, as a fundamental rule, the motor stops.

The stepper motor can have some advantages for acceleration of the drive, in particular the linear drive. Although the maximum speed of stepper motors is certainly lower than that of servo motors, and the transmissible torque certainly also decreases with the increasing speed of stepper motors, an expected, required speed can probably be lower than 100 revolutions per minute. The shorter overall length and the lower weight of the stepper motor are certainly advantages that help in a design to make the transverse runner, particularly the transverse axis, smaller and lighter. Programming and controlling a stepper motor is certainly easier than programming and controlling a servo motor.

The 1.8° increments can result in 200 steps for one complete revolution of the motor. A route can be measured and thus programmed very well, without any need for additional monitoring, for example by limit switches or encoders. The stepper motor certainly also has good conditions in terms of costs. This helps to keep the overall costs of the particle counting device low, since the costs of a stepper motor are certainly far lower than those of a servo motor.

Another definite advantage of the stepper motor is a standardised flange size. The connection dimensions for stepper motors are basically regulated according to the National Electrical Manufacturers Association (NEMA) standard and are thus the same depending on size. The name of the stepper motors can generally be used to determine their flange size, for example, a stepper motor with the name NEMA 17 has a flange size of 42 mm, or a NEMA 23 stepper motor has a flange size of 57 mm. This standardisation has the basic advantage that motors from different manufacturers can usually be exchanged without any design changes. Stepper motors with the same flange size but different torque can also be exchanged.

One task of the transverse runner can be to guide the probe for particle measurement transversely, in particular orthogonally, to the transport direction of the conveyor belt. The probe can be mounted on the guide carriage or on the mounting bracket. The probe holder can be configured to be additionally guided through the rack. The probe holder can, in particular, be made of POM. The spur gear can also be made of POM, as stated above. This makes it possible to minimise sliding friction between the rack and the probe holder. Furthermore, weight savings can be achieved. The probe holder and the mounting bracket can be connected to one another in particular by means of a screw connection. The probe holder can have the groove into which the probe can be inserted and clamped with the clamping plate. The probe holder can, particularly for safety reasons, be configured in such a way that the probe holder surrounds the spur gear, in particular completely. This can avoid other elements being trapped between the spur gear and the rack when the spur gear is moving.

For design of the transverse runner, tunnel types of sterilisation tunnel can be analysed and important dimensions for the scanner, in particular for the transverse runner, can be incorporated. The most important dimensions are basically a maximum height of the sterilisation tunnel and a width of the conveyor belt. The sterilisation tunnel can in particular have a conveyor belt width of 600 mm to 800 mm. Furthermore, the sterilisation tunnel can have a maximum height of 160 mm to 230 mm. Furthermore, there can be between 1 and 5 particulate filters in the sterilisation tunnel. The particulate filter can have a filter length of 250 mm to 580 mm and a filter width of 600 mm to 720 mm. The dimensions listed can, in particular, be taken from existing technical drawings of the sterilisation tunnel and/or the sterilisation tunnel can be measured in situ. The maximum height and the maximum width of the transverse runner of the scanner can certainly be derived from these dimensions. The maximum height, in particular passage height, of 160 mm basically sets a limit on the overall height. In addition, the maximum overall height can be limited by separating bulkheads between different zones in the sterilisation tunnel. This may also require the particle counting device to be as flat as possible. However, it is a basic goal to make the height of the particle counting device as low as possible in order to facilitate handling on the sterilisation tunnel and the introduction of the particle counting device onto the conveyor belt. The transverse runner can certainly be adjusted to the width of the conveyor belt. Due to the different widths of the particulate filters, the scanner can have a modular design. The transverse runner can be constructed in such a way that the transverse runner can be adapted to a tunnel width of the sterilisation tunnel. In particular, a length of the rack, a length of the guide rail and a length of the base plate can be adjusted. The remaining components of the particle counting device can be independent of the tunnel type of the sterilisation tunnel. For example, a height of the transverse runner can be 80 mm and a total weight of the transverse runner can be 1870 g. In addition, connections of components of the transverse runner can have Phoenix plug-in terminals. This can contribute to a further reduction in the overall height of the particle counting device, in particular by up to 10 cm.

As stated above, the scanner comprises the bogie. The term “bogie” as used herein is a broad term that should be given its ordinary and common meaning as understood by those skilled in the art. The term is not limited to a specific or adapted meaning. The term can, without limitation, refer in particular to any part of a device which is configured to carry further components of the device. The bogie can therefore be configured as a supporting part of the device. The bogie can, in particular, have elements which enable movement of the bogie on a surface. The elements can, in particular, include one or more wheels, one or more wheel suspensions, at least one drive and/or at least one motor. The bogie can therefore also be referred to as the chassis.

The bogie can have a frame. The frame can also be referred to as the base frame. The frame can in particular be made from a metal sheet, in particular from a metal sheet made of austenitic stainless steel. The metal sheet can, in particular, have a thickness of 1 mm to 5 mm, preferably 1.5 mm to 2.5 mm and particularly preferably 2 mm. Due to its structure, the austenitic stainless steel can generally be cold-formed very well, so that the metal sheet can be bent. Thanks to a chromium content of >13.5%, the austenitic stainless steel also has good corrosion resistance and is therefore well suited for use in the pharmaceutical sector. Through well-chosen folds, a high level of sheet metal rigidity can be achieved. By welding abutting edges, the rigidity of the sheet metal can be further increased. In this way, a torsion-resistant and lightweight frame can be produced, on which further components can be mounted. Alternatively or additionally, the frame can be at least partly made of aluminium. This can contribute to reducing the weight of the particle counting device. As stated above, the transverse runner is mounted on the bogie. In particular, the transverse runner, in particular the base plate of the transverse runner, can be mounted on the frame. In particular, the transverse runner can be mounted centrally on the bogie, in particular on the frame. Furthermore, the transverse runner can be mounted to be flush with the bogie, in particular with the frame. In particular, the transverse runner can be mounted to be flush with a rear side of the bogie, in particular of the frame. Due to the small run-off surfaces before and after the sterilisation tunnel, the size can be minimised and particle counting can be carried out even without run-off zones.

The controller can in particular be mounted, in particular installed, in the middle of the bogie. The controller can in particular be mounted on a free area of the bogie. A length of the free area can, in particular, be selected such that the controller and also cable ducts, in particular for wiring, can be mounted.

As stated above, the bogie is configured to move the linear guide in the transport direction of the conveyor belt. For example, the bogie can have a crawler chassis which is configured to move the linear guide in the transport direction of the conveyor belt. However, the bogie can preferably have at least two wheels, in particular at least two drive wheels, which are configured to move the linear guide in the transport direction of the conveyor belt. A drive wheel can, in particular, be any wheel that is configured for the independent movement of a device on which the drive wheels are mounted. The drive wheels can in particular, be driven by a motor, as is explained in more detail below.

As stated above, the conveyor belt of the sterilisation tunnel can have a wire mesh. As is explained in more detail below, the wheels can, in particular, be constructed in such a way that a contact area between the wheels and the wire mesh of the conveyor belt is increased. In particular, through a suitable choice of material and the particle counting device's own weight, sufficient rolling friction can be applied to achieve slip-free movement.

As the wire mesh is usually made of at least one metal, metal-to-metal contact can be categorically avoided in the design and material selection of the wheels, as otherwise, due to a generally small contact area on the wire mesh, not enough rolling friction can generally occur between the wheels and the conveyor belt and slip-free movement basically cannot be guaranteed. A similar behaviour is assumed if the wheels are made of a thermoplastic or a thermosetting material. Here, too, there may well not be sufficient rolling friction between the material of the wheels and the conveyor belt to ensure reliable, slip-free movement.

In particular, the wheels can be at least partly made of an elastomer. The elastomer can be selected from a group consisting of: silicone, ethylene propylene diene (monomer) rubber (EPDM). These materials are approved for use in the pharmaceutical sector. However, other elastomers are certainly also conceivable. In particular, the wheels can each have one or more O-rings which are made from the elastomer. The elastomer can be configured to generate rolling friction between the wheels and the conveyor belt. Due to elastic deformability and a high coefficient of friction of the elastomers, a contact area between the O-rings and the conveyor belt can be increased. This means that slip-free operation can be possible.

The wheels can, in particular, be designed in such a way that the O-rings made of the elastomer can be mounted thereon. The wheels, in particular the drive wheels, can, in particular, be made of polyoxymethylene (POM). In particular, the wheels can have one or more grooves which are configured to receive the O-rings. A diameter of the wheels can be selected such that a low ground clearance is achieved between the frame and the conveyor belt. This means that the later overall height of the scanner can be kept to a minimum. The wheels, in particular the drive wheels, can each have a plurality of O-rings, in particular at least two, preferably at least three, preferably at least four O-rings, particularly in order to increase the contact area between the wheels and the conveyor belt. The O-rings can be arranged spaced apart from one another on at least one peripheral surface of the drive wheels. The O-rings can each be received in grooves on the peripheral surface of the drive wheels.

The drive wheels can each be mounted on an axle. A rotational movement of an axle can be transmitted positively to the drive wheels using feather keys. This can prevent the wheels from spinning. In addition, the drive wheels can be secured with a set screw, in particular to prevent them from moving on the axle.

The particle counting device can further comprise at least a second motor selected from the group consisting of: a stepper motor, a servo motor. However, other types of motors are certainly also conceivable. The second motor can be configured to drive at least one of the drive wheels. For further details on the design of the stepper motor and the servo motor, reference can be made to the above description.

Preferably, the second motor can be a stepper motor. In particular, the second motor can be a motor with a size that corresponds to a size of the first motor. In particular, the second motor can be a motor which is structurally identical to the first motor. This minimises the number of different components of the particle counting device. Furthermore, just one type of stepper motor can be kept in stock as a spare part, which generally ensures interchangeability.

The second motor can, in particular, comprise a second motor driver. The second motor driver can be configured to transmit signals to the second motor and/or to supply the second motor with a voltage. For this purpose, the second motor driver can be connected to a mains adaptor, for example. The mains adaptor can be configured to convert a voltage to a voltage required by the second motor, for example, the mains adaptor can be configured to convert a voltage from 230 V to 24 V. In particular, the particle counting device can comprise a mains adaptor which is configured to supply the first motor, the second motor, the first motor driver and the second motor driver. Alternatively, two mains adaptors can also be provided, each of which supplies the first motor and the first motor driver or the second motor and the second motor driver.

In particular, the second motor can be a NEMA 17 stepper motor (Stepperonline, China) with a related stepper motor driver. Despite its small size, the Nema17 stepper motor can be suitable for generating the power required to drive the movement of the bogie. For further details, reference is made to the description above. Furthermore, the second motor can be a stepper motor of the OMC Stepperonline 17HS24-2104S type with a step generator of the OMC Stepperonline DM556N type. The bogie can, in particular, have a drive axle. The stepper motor can be mounted on the base frame to be offset transversely, in particular at 90°, from the drive axle. The bogie can also have several bevel gears. In order to transfer the rotational movement of the stepper motor to the drive axle, bevel gears with a 2:1 transmission ratio can be selected. A first bevel gear can, for example, have a number of teeth z of 15 and a second bevel gear can have a number of teeth z of 30. The first bevel gear and the second bevel gear can each be made of POM and have a module m of 1.

The first bevel gear can be configured to be driven by the second motor and to transmit a rotational movement to a drive axle via the second bevel gear. The transmission ratio allows the speed n₁ of the second motor on the drive axle to be halved and the transmitted torque M₁ to be doubled. As a result, a stepper motor with a small holding torque can generally be used. z₁ corresponds to a number of teeth on the first bevel gear and z₂ a number of teeth on the second bevel gear.

$${n}_{2} = \frac{{n}_{1} * {z}_{1}}{{z}_{2}} \qquad ( 15 )$$

$${n}_{2} = \frac{{n}_{1} * 15}{30} = {n}_{1} * 0.5 \qquad ( 16 )$$

$${M}_{2} = {M}_{1} * \frac{{z}_{2}}{{z}_{1}} \qquad ( 17 )$$

$${M}_{2} = 0.65 Nm * \frac{30}{15} = 1.3 Nm \qquad ( 18 )$$

A torsionally rigid but angularly and transversely flexible compensating coupling can be used to transmit the torque and speed from the second motor to the shaft of the first bevel gear. This can, in principle, compensate for tolerances or misalignments in the design. A compensating coupling used can generally be backlash-free and torsionally rigid and can compensate for both radial and axial angular misalignment. Gear shafts can be held in a suitable position by a holder. Grooved ball bearings can be pressed into the holder, which support the shaft of the first bevel gear and the axle and ensure smooth running. Grooved ball bearings have the basic advantage that they do not have an increased friction torque when warming up. They also show generally little wear at low speeds and are maintenance-free. The first bevel gear can be friction-locked on the shaft using a set screw. The second bevel gear can also be mounted on the axle with a set screw and can also be secured with an adjusting ring to prevent accidental displacement on the shaft.

Furthermore, the wheels can include one or more, in particular two, rear wheels. The rear wheels can be designed to be drive-free. The rear wheels can, in particular, run smoothly. For further details of the design of the rear wheels, reference is made to the description above. The rear wheels can be made of POM. Furthermore, the rear wheels can have grooves which are configured to receive O-rings. The O-rings can be made of at least one elastomer. A diameter of the rear wheels can be selected such that the rear wheels do not represent an obstacle to the movement of the transverse runner. Ball bearings can be pressed into the rear wheels, in particular for smooth running. The rear wheels can have a locking ring which is configured to secure the ball bearing against unintentional loosening. The rear wheels can each be pushed on a shaft and mounted with a self-locking nut. The self-locking nut can be configured to prevent the self-locking nut from loosening when the rear wheel rotates. The shafts of the rear wheels can also be mounted, in particular screwed, on the bogie with self-locking nuts.

The bogie can be designed to be used in all types of sterilisation tunnels. Thanks to its compact size, the bogie is easy to handle. The frame can have welding studs, in particular welding studs arranged at a rear end, which can be configured in particular for mounting the transverse runner. The transverse runner can be detachably connected to the bogie using knurled nuts. It may be possible to easily replace the transverse runner. A height of the bogie can in particular be chosen such that it does not exceed a total height of the particle counting device of 160 mm. Furthermore, the overall height of the bogie can be kept as low as possible. The bogie with the above components can, for example, have a total height of 79 mm and a total weight of 3000 g. The bogie can have a width of 300 mm, for example. This can make it easier to handle and introduce the particle counting device into the sterilisation tunnel.

As stated above, the scanner further comprises the at least one controller which is configured to control the movement of the scanner. The movement can, in particular, be a movement of the bogie in the transport direction of the conveyor belt. Furthermore, the movement can involve guiding the probe holder transversely to the transport direction of the conveyor belt. The term “controller” as used herein is a broad term that should be given its ordinary and common meaning as understood by those skilled in the art. The term is not limited to a specific or adapted meaning. The term can, without limitation, refer in particular to a one-part or multi-part device of the particle counting device, which is configured to completely or partially control and/or regulate an operation of the particle counting device. In particular, the controller can include a programmable logic controller (PLC). In the context of the present invention, a “programmable logic controller (PLC)” basically means any device that is used to control or regulate a machine or system and is programmed on a digital basis. In particular, the controller can be configured to control and/or regulate the motor of the drive for guiding the probe holder transversely, in particular essentially perpendicularly, to the transport direction of the conveyor belt and/or the motor of the drive for moving the bogie along the transport direction of the conveyor belt. The controller can, in particular, comprise at least one data processing device, for example at least one processor. Accordingly, the controller can be implemented partly by hardware and/or, alternatively or additionally, completely or partly by software. Furthermore, the controller can include at least one volatile and/or non-volatile data memory. The controller can, in particular, be configured to control at least one of the drives. In the context of the present invention, “controlling a drive” is basically to be understood as a type of operation of the drive, in particular starting and/or stopping a movement or steps and, in particular, a change in the speed of the movement. The controller can, in particular, be configured to control the drives during operation of the particle counting device in such a way that repeatedly predeterminable movements of the bogie or guides of the probe holder are carried out with the linear guide. The controller can, in particular, be configured to transmit signals to the first motor driver of the first motor or signals to the second motor driver of the second motor. The controller can, in particular, be configured by programming, for example to control the particle counting method, which is explained in more detail below. The controller can also be configured to log data from the drives. Logging can, in particular, include saving or recording the data. In particular, the controller can include a TIA PLC S7-1200. Despite its small size and a limited number of channels, the TIA PLC S7-1200 can provide sufficiently high performance for the controller. Furthermore, the controller can include a Siemens PLC S7-1211C DC/DC/DC, and, in particular, an 8″ touch panel for operation. The Siemens PLC S7-1211C DC/DC/DC can, in particular, have a compact design. Furthermore, the controller can include a board with a microcontroller (Arduino), which can be connected to a computer, particularly via an interface, particularly via a USB interface. A program for controlling the movement of the bogie and/or guiding the probe holder may be loaded on the board. The program can, in particular, specify steps, a speed and/or a direction of rotation of the motors, in particular of the stepper motors.

Furthermore, the particle counting device can have at least one y position sensor for determining a position of the probe in one dimension in the transport direction on the conveyor belt. Furthermore, the particle counting device can have at least one x position sensor for determining a position of the probe in a dimension transverse to the transport direction, in particular essentially perpendicular to the transport direction, on the conveyor belt. The term “position sensor” generally refers to any sensor that is configured to measure a distance between an object and a reference point and/or changes in length. The position sensor can, in particular, be configured to convert a change in a path into a standard signal or to transmit it to a control device. The y position sensor can be connected to the bogie, in particular to the drive of the bogic. As stated above, the bogie can have the stepper motor and the y position sensor can include an incremental encoder of the stepper motor. The x position sensor can be connected to the linear guide, in particular to the drive of the linear guide. As stated above, the linear guide can have the stepper motor and the x position sensor can include an incremental encoder of the stepper motor. The particle counting device, in particular the controller, can be configured to count pulses of the stepper motor, in particular of the stepper motor driver, in particular by means of a forward and reverse counter. The forward and reverse counter can, in particular, be part of the controller. This means that any positions that may be conspicuous in the measurement values can be cached in a Cartesian manner during the measurement and can be approached in manual mode in a subsequent, more precise check, as is explained in more detail below.

In particular, the particle counting device, in particular the controller, can be configured to count first pulses of the first motor, in particular of the first stepper motor driver, by means of at least one first forward and reverse counter. Furthermore, the particle counting device, in particular the controller, can be configured to count second pulses of the second motor, in particular of the second stepper motor driver, by means of at least one second forward and reverse counter. In particular, the particle counting device, in particular the controller, can be configured to determine a position of the probe on the linear guide using the first pulses of the first motor and a position of the bogie on the conveyor belt using the second pulses of the second motor. In addition, the particle counting device, in particular the controller, can comprise at least one further forward and reverse counter, which is configured to count pulses of the second motor, in particular of the second stepper motor driver. The further forward and reverse counter can therefore be configured in particular to determine a path distance during a movement in the transport direction. The particle counting device, in particular the controller, can be configured to reset the further forward and reverse counter to zero after a stepwise movement of the bogie, in particular after reaching a next measuring path. Furthermore, the linear guide can have a first end stop and a second end stop and the controller can be configured to reset the first forward and reverse counter to zero when the probe is at the first end stop. In particular, the first end stop can be a left end stop.

In particular, since the first forward and reverse counter, the second further forward and reverse counter and the further forward and reverse counter are each configured to count pulses of the first stepper motor driver and the second stepper motor driver respectively, the controller can be configured to use the pulses of the first stepper motor driver or the second stepper motor driver to calculate metric data for a representation of coordinates.

To determine a position of the probe holder transverse to the transport direction, the following basically applies:

$$Pulse per revolution = π * {diameter}_{g e a r} \qquad ( 19 )$$

Thus, a ratio between pulses and Cartesian positions can be specified:

$$\frac{{x}_{Position ; Cartesian}}{{x}_{P o sition ; Pulse}} = \frac{π * {Diameter}_{g e a r}}{Pulse per revolution} \qquad ( 20 )$$

A Cartesian position can now be determined by rearranging:

$${x}_{Position ; Cartesian} = \frac{π * {Diameter}_{gear}}{Pulse per revolution} * {x}_{Position ; Pulse} \qquad ( 21 )$$

The pulse per revolution can depend on a speed as well as on the smallest possible adjustable time delay in the pulse programming. If you use a minimum time delay of 1 ms and a speed of 5 cm/s, the geometry of the driving gear results in:

$$Pulse per revolution = \frac{π * {Diameter}_{g e a r}}{2 * Speed * Pulse length} \qquad ( 22 )$$

$$Pulse per revolution = \frac{π * 19 mm}{2 * 50 \frac{mm}{s} * 0.001 s} = 596.90 \qquad ( 23 )$$

For the Cartesian position, the following results:

$${x}_{P o s i t i o n ; Cartesian} = \frac{{x}_{P o sition ; Pulse}}{6.70126 \frac{1}{mm}} \qquad ( 24 )$$

Since a bevel gear transmission, in particular a single-stage bevel gear transmission, can be installed for propulsion, as explained above, a transmission ratio can be taken into account:

$$n = \frac{{Diameter}_{g e a r}}{{Diameter}_{P i n i o n}} \qquad ( 25 )$$

With a pinion diameter of 11.96 mm, this can result in:

$$Pulse per {revolution}_{Wheel} = Pulse per {revolution}_{Motor} * n \qquad ( 26 )$$

$$Pulse per {revolution}_{Wheel} = Pulse per {revolution}_{Motor} * 1.58824 = 635.294 \qquad ( 27 )$$

To determine a position of the scanner in the transport direction, the following results:

$${y}_{Position ; Cartesian} = \frac{π * {Diameter}_{gear}}{Pulse per revolution * n} * {y}_{Position , Pulse} \qquad ( 28 )$$

$${y}_{Position ; Cartesian} = \frac{{y}_{Position ; Pulse}}{2.55975 \frac{1}{mm}} \qquad ( 29 )$$

Furthermore, the scanner can have one or several limit switches. The term “limit switch” generally refers to any device that is configured to record when a moved object has reached a defined position. In particular, the transverse runner can have at least two limit switches, in particular at least two roller limit switches, which are configured to determine the positions of the limits of the probe transverse to the transport direction of the conveyor belt. In particular, the transverse runner can have at least two snap switches, for example at least two Marquardt 1006.1501 snap switches. Furthermore, the bogie can have at least one limit switch, in particular at least one spring rod limit switch, for determining at least one position of at least one limit of the particle counting device in the transport direction.

Furthermore, the particle counting device can comprise at least one stationary user interface. The user interface can be connected to the scanner. A movement of the scanner can be controllable using the user interface. The term “interface” as used herein is a broad term that should be given its ordinary and common meaning as understood by those skilled in the art. The term is not limited to a specific or adapted meaning. The term can, without limitation, refer in particular to a device of basically any design, which is configured to receive at least one piece of information and then optionally process and/or forward it completely or partially, for example to at least one controller. The term “user interface” as used herein is a broad term that should be given its ordinary and common meaning as understood by those skilled in the art. The term is not limited to a specific or adapted meaning. The term may, without limitation, refer in particular to any interface for inputting commands and/or outputting information, and/or to a wireless or wired interface for the unidirectional or bidirectional exchange of data and/or commands between a device and at least one operator of the device. The user interface can be a communication interface, in particular a data interface, which is configured to receive data from another device and/or from a user and/or to transmit data from the user interface to external devices. The user interface can have at least one electronic interface and/or a human-machine interface, such as an input/output device such as a display, in particular a touch display, in particular an 8″ touch display, and/or a keyboard. The interface can have at least one data connection, for example a Bluetooth connection, an NFC connection or another connection. The user interface can have at least one network or be part of a network. The user interface may have at least one Internet port, at least one USB port, at least one drive, or a web interface.

In particular, the user interface can have a graphical user interface, in particular a graphical user interface with at least one touch screen. The term “graphical user interface” as used herein is a broad term that should be given its ordinary and common meaning as understood by those skilled in the art. The term is not limited to a specific or adapted meaning. The term can, without limitation, refer in particular to a form of a computer user interface which has the task of making application software on a computer operable using graphic symbols or control elements. This can be done, for example, using a mouse as a control device, with which the graphic elements are operated or selected. Alternatively or additionally, the operation can be carried out by touching a sensor screen, in particular a touch screen.

In particular, at least one travel path and/or at least one measuring position for particle counting can be predeterminable using the user interface. Furthermore, the at least one travel path and/or the at least one measuring position can be predeterminable in manual operation or in automatic operation. Furthermore, at least one speed can be predeterminable for the at least one travel path, in particular in manual mode, as is explained in more detail below. Furthermore, the user interface can be connected to the particle counter.

The graphical user interface can, in particular, comprise at least three operator interfaces, in particular at least three individual operator interfaces. The operator interfaces can each be accessed via a main menu. The main menu can also be called the basic screen. The graphical user interface can be configured in such a way that the three operator interfaces, which can also be referred to as subordinate menu items, can be reached from the main menu, in particular through a TIA-internal function, which can be referred to as “ActivateScreen”. A first operator interface can correspond to an automatic, in particular to a partially or fully automatic operation. The first operator interface can in particular include starting and stopping a defined, automatic run of the bogie and/or the probe. Furthermore, the first operator interface can include a coordinate display and/or coordinate storage. Partially or fully automatic operation can also be referred to as par-measuring operation. A second operator interface can correspond to manual operation, in particular hand operation. The second operator interface can in particular provide a separate control option for directions of movement, in particular directions of movement of the bogie and/or the probe holder. Furthermore, the second user interface can provide a run in a starting position. In addition, the second operator interface can provide one or more service functions, which can include, for example, a reset of increments or a display of digital outputs. A third operator interface may correspond to an interface for system settings. The at least three operator interfaces can each include a display of a date and a time, which can in particular be picked up from a control-internal system time. Furthermore, the at least three operator interfaces can each be configured to display occurring faults via a message text line. In particular, the third operator interface can be configured to end a runtime and, in particular, to then go to a settings area of the user interface. Furthermore, the third operator interface can comprise user management, in particular user account management. The user interface can be configured in particular to create groups for operators and administrators, in particular with respective users and their initial passwords, in particular via the TIA-internal function, in particular in advance of particle counting. One or more functions, which may only be controlled or activated by a limited group of users, can be provided with a safety function, in particular a security function, with a corresponding restriction of authorisation to a preset group.

As stated above, the second operator interface can in particular provide a separate control option for directions of movement, in particular directions of movement of the bogie and/or the probe holder. The second operator interface can be configured for manual driving of the scanner, in particular for manual guiding of the probe holder transversely to the transport direction of the conveyor belt and/or for manual driving of the bogie in the transport direction of the conveyor belt. The second operator interface can in particular have several buttons, in particular several individual buttons, which are configured to control the first motor of the linear guide and/or the second motor of the bogie, in particular in a direction of travel. In particular, the buttons can include at least a first button for controlling a forward movement of the bogie in the transport direction. Furthermore, the buttons can include at least a second button for controlling a backwards movement of the bogie contrary to the transport direction. Furthermore, the buttons can include at least a third button for controlling the probe holder transversely to the transport direction, in particular from the first end of the guide rail to the second end of the guide rail. Furthermore, the buttons can include at least a fourth button for controlling the probe holder transversely to the transport direction, in particular from the second end of the guide rail to the first end of the guide rail. Guiding the probe holder from the first end of the guide rail to the second end of the guide rail can also be referred to as moving or guiding the probe holder to the left. Furthermore, guiding the probe holder from the second end of the guide rail to the first end of the guide rail can also be referred to as moving or guiding the probe holder to the right, or vice versa. The first button, the second button and the fourth button can in particular be arranged as a D-pad. Furthermore, the second operator interface can include a home button, which can in particular be positioned in the centre of the D-pad. The home button can be used to guide the probe holder to the first end of the guide rail or to the second end of the guide rail.

In particular, the second operator interface can be configured to move the scanner back to a starting position after the particle counting method has been carried out, in particular to move the bogie back to the starting position. In particular, moving the bogie back to the starting position can include a backwards movement of the bogie into the starting position. For this reason in particular, the second operator interface can include at least one, in particular at least two, input fields for a speed. In particular, the second operator interface can include a first input field for a speed of the bogie in the transport direction and a second input field for a speed of the probe holder transverse to the transport direction. This means that the scanner can, if necessary, be moved back to the starting position more quickly than the scanner has carried out the particle counting method. Input speed values can be configured to manipulate holding times of on-switch delays or off-switch delays, which can be used for pulse generators, which will be explained in more detail later. Furthermore, the second operator interface can have at least one “back” button, by means of which the second operator interface, in particular the manual mode, switches to the main menu with a screen change and in particular ends the manual mode. Furthermore, the second operator interface can have one or more information windows, particularly as one or more test runs, for example for troubleshooting, can be carried out via the second operator interface. The information window can in particular have one or more texts, displays and buttons. The second operator interface can in particular have a service button by means of which the information window is activated. Activation can be realised in the programming in particular by means of a visibility function for screen elements. In particular, the service button can be configured to activate a bit that is used for a visibility query of elements. The information window can in particular have a close button, which can in particular be configured to deactivate the bit, whereby the elements lose their visibility. The information window can also include one or more buttons for resetting increments for the movement of the bogic and/or for guiding the probe holder. In addition, the information window can have one or more buttons for simulating end stops. In addition, the information window can have one or more displays for binary outputs of the step generator controller. Other configurations are certainly also conceivable.

As stated above, the first operator interface can in particular include starting and stopping a defined, automatic run of the bogie and/or the probe. In particular, the first operator interface can include a method for a predefined meandering path of the scanner. In particular, the travel path can comprise a meandering pattern with alternating movements of the probe transverse and parallel to the transport direction. In particular, the user interface can be configured to move the linear guide stepwise in the transport direction of the conveyor belt by means of the bogic. Furthermore, the user interface can be configured to guide the probe holder transversely, in particular essentially perpendicularly, to the transport direction by means of the transverse runner. The stepwise movement of the linear guide in the transport direction and the guidance of the probe holder transversely to the transport direction, in particular from the first end of the guide rail to the second end of the guide rail or from the second end of the guide rail to the first end of the guide rail, can take place alternately, so that the meandering pattern is created. The first operator interface can in particular include a start button which is used to start a programmed sequence of steps. Furthermore, the first operator interface can be configured to make a stop button visible, which appears in particular at a position of the first operator interface that corresponds to a position of the start button. Furthermore, the first operator interface can be configured to stop the programmed sequence of steps via the stop button and in particular to deactivate the visibility of the stop button. Furthermore, the first operator interface can have two output fields, which display a current Cartesian position of the probe. Furthermore, the first operator interface can have a “Save position” button. The first operator interface can be configured to temporarily store coordinates recorded by pressing the “Save position” button, in particular coordinates recorded at the time of pressing, in particular in a data block. This allows a counter, particularly a leakage counter, to increase a number of possible leaks. A representation of positions of possible leaks can have several, in particular five, variables, each for a position in the transport direction and for a position transverse to the transport direction. The positions can also be referred to as x and y positions. Values of these variables can be zero by default, especially if the leakage counter has a value of zero. As soon as the leakage counter is increased to the value of one, current position values will be displayed in a first pair of variables, in particular a first pair of x-y variables. However, this would certainly lead to the problem that position values of a first leak would continue to run simultaneously to a current measuring position instead of taking a snapshot of the position. This problem can be solved by using flanks. Here, as soon as the leakage counter increases to the value one, a pulse, in particular a pulse with a time span of 100 ms, can be started, which is configured so that the current position values are only written during the time span of the pulse. The representation of positions of possible leaks can in particular have a “clear” button, which is configured to reset the variables to the value zero, in particular by resetting the leak counter. As already stated, storage of up to 5 leaks can be provided in particular, since this number of abnormalities indicates a critical condition of the filter element being checked. The user interface can be configured to specifically move the probe to at least one predeterminable probe position, in particular to a stored position, and to carry out a particle count there. In this way, the saved positions of possible leaks can be checked again.

As stated above, the probe can include the probe opening, in particular the probe funnel. The user interface can be configured, in particular via the first operator interface, to carry out the stepwise movement of the linear guide in the transport direction of the conveyor belt by means of the bogie in such a way that an increment of the particle counting device in the transport direction of the conveyor belt by means of the bogie is smaller than the outer diameter of the probe opening. Further details can be found in FIG. 16 and the associated description given below it. Furthermore, the user interface can be configured to record and in particular display particle counts depending on a probe position.

Furthermore, the particle counting device can comprise at least one temperature sensor. The temperature sensor can be configured to record a temperature in the sterilisation tunnel. In particular, the temperature sensor can be configured to record a temperature of air in the sterilisation tunnel. The temperature sensor can in particular be an electrical or electronic component, which can be configured to provide an electrical signal as a measurement of the temperature.

The temperature sensor can, in particular, be mounted on the bogie. The particle counting device, in particular the user interface, can be configured to issue a warning when at least one temperature threshold, in particular a defined temperature threshold, is exceeded, in particular in the first operator interface and/or in the second operator interface. Furthermore, the particle counting device, in particular the user interface, can be configured to interrupt a movement of the linear guide in the transport direction of the conveyor belt by the bogie when the at least one temperature threshold is exceeded.

In a further aspect of the present invention, a sterilisation tunnel of a medicament filling system is proposed. The sterilisation tunnel comprises at least one conveyor belt. The conveyor belt is configured to guide at least one vessel along a transport direction of the conveyor belt. Furthermore, the sterilisation tunnel includes at least one particulate filter. Furthermore, the sterilisation tunnel comprises at least one particle counting device arranged between the particulate filter and the conveyor belt, as has already been described or is described in the following. In particular, the particle counting device can be arranged at least partially on the conveyor belt. The probe of the particle counting device has a probe opening, in particular a probe funnel, facing the particulate filter.

The sterilisation tunnel can also include at least one supply air duct. The term “supply air duct” as used herein is a broad term that should be given its ordinary and common meaning as understood by those skilled in the art. The term is not limited to a specific or adapted meaning. The term can, without limitation, refer in particular to a device which is configured to direct supply air comprising a gaseous medium, in particular air, in such a direction that the supply air can be supplied to another device. The supply air duct can be configured to supply the supply air to the sterilisation tunnel, in particular to the particulate filter. The supply air duct can, in particular, supply the air surrounding the medicament filling system to the sterilisation tunnel. The supply air duct can be configured to supply the supply air to the other device in a laminar flow.

The supply air duct can include at least one fan to suck in ambient air. The term “fan” as used herein is a broad term that should be given its ordinary and common meaning as understood by those skilled in the art. The term is not limited to a specific or adapted meaning. The term can, without limitation, refer in particular to a device which is configured to convey a gaseous medium by increasing the pressure in the gaseous medium. The fan can, for example, comprise an axially or radially rotating propeller, which increases the pressure in the gaseous medium by means of this very rotation. The fan can have a suction side and a pressure side, wherein the pressure in the gaseous medium on the pressure side can be greater than on the suction side. The gaseous medium can be conveyed in particular from the suction side to the pressure side of the fan. The gaseous medium conveyed by the fan can include air, in particular the air surrounding the medicament filling system. The fan may be selected from the group consisting of: an axial fan; a diagonal fan; a radial fan; a centrifugal fan; a tangential fan; a cross-flow fan.

The sterilisation tunnel can further comprise at least one suction device. The suction device can be configured to suck out air below the conveyor belt. The term “suction device” as used herein is a broad term that should be given its ordinary and common meaning as understood by those skilled in the art. The term is not limited to a specific or adapted meaning. The term can, without limitation, refer in particular to a device which is configured to partially remove a gaseous medium, in particular air, from a removal area and to supply it to an output area. The partial removal can, in particular, include removal of only a partial volume of the gaseous medium in the removal area, wherein the partial volume removed is dependent on an extraction rate of the suction device. The removal area can be at least partly surrounded by another device or can at least partly coincide with this other device. The suction device can thus transport the gaseous medium, for example air, from this other device, in particular by removing the gaseous medium from the removal area and supplying it to the output area. For example, the removal area of the suction device can at least partly coincide with the sterilisation tunnel, wherein the output area can be located outside the sterilisation tunnel, so that the suction device can convey air out of the sterilisation tunnel.

The sterilisation tunnel can, in particular, comprise at least three zones. A first zone can be a warm-up zone. The warm-up zone can also be referred to as the inlet. The second zone can be a sterilisation zone. The sterilisation zone can also be referred to as the hot part. A third zone can be a cooling zone. The third zone can also be referred to as the cooling zone. The conveyor belt can be configured to move, in particular at a constant speed, through the sterilisation tunnel.

The warm-up zone can be configured to slowly bring the vessels up to a temperature of the sterilisation zone. In particular, the warm-up zone can be configured to heat, in particular to heat slowly, the glass of the vessels, in particular to reduce tensions that arise in the glass and to avoid possible glass breakage. The sterilisation zone can be configured to vaporise water on and in the vessels. Furthermore, the sterilisation zone can be configured to sterilise the vessels, in particular at a temperature of over 300° C., preferably at 330° C. The cooling zone can be configured to cool, in particular to cool slowly, the vessels, in particular to a temperature of less than or equal to 60° C., in particular in order to reduce built-up tensions in the glass in a controlled manner. The medicament system can be configured to transport the vessels from the cooling zone to the filling system. The filling system can be configured to fill the vessels with one or several medicaments. Furthermore, the filling system can be configured to seal the vessels after filling. The inspection machine can be configured to examine the filled medicaments for contamination, in particular particles. The inspection machine can, in particular, be configured for a visual inspection of the medicaments. If no contamination is found, the medicament is released for packaging.

The warm-up zone can have at least a first supply air duct, at least a first fan and a first particulate filter. The first fan and the first particulate filter can be arranged in the supply air duct. The first particulate filter can also be referred to as a prefilter. The first fan can be configured to suck in ambient air, in particular ambient air from the clean room, through the first particulate filter. The first fan can also be configured to supply the sucked-in ambient air to the first particulate filter. The supply air duct can be configured to provide the filtered ambient air as a laminar flow. The filtered air can flow through a conveyor belt area of the sterilisation tunnel.

The suction device can include at least one further fan. The further fan can, in particular, be arranged below the conveyor belt. The further fan can be configured to suck out air, in particular moist air, below the conveyor belt.

The sterilisation zone can work in a circulating procedure. The sterilisation zone can have at least one second fan and at least one second particulate filter. The second fan can be configured to suck out air from below the conveyor belt and direct it into an area above the conveyor belt. The sterilisation zone can also have at least one heating unit which is configured to heat the air. The second fan can also be configured to supply the heated air, in particular hot air, to the second particulate filter. The hot air can emerge, in particular in a laminar manner, above the conveyor belt area.

The further fan can be configured to suck out air, in particular air saturated with water vapour, in the sterilisation zone, in particular to prevent the air saturated with water vapour from accumulating in the sterilisation zone. The sterilisation zone can also have at least a third particulate filter. If necessary, ambient air can flow in via the third particulate filter.

The cooling zone can have at least a third fan and at least a fourth particulate filter. The third fan can be configured to suck in ambient air, in particular cool ambient air, in particular cool clean room air, and to push it through the fourth particulate filter. The emerging laminar flow can be configured to cool the vessels.

The sterilisation tunnel can also have at least a fourth fan and at least one drain channel. The fourth fan can in particular be a fan of the cooling zone. The fourth fan can be configured to suck out air, in particular heated air, in particular air in the cooling zone, below the conveyor belt and to supply it to the exhaust air duct.

The laminar air flow in the individual zones, in particular in the warm-up zone, the sterilisation zone and the cooling zone, can flow from top to bottom. Any particles present in the sterilisation tunnel are thereby pushed downwards, which definitely ensures that no particles enter the vessels. In the sterilisation tunnel, particularly in the entire sterilisation tunnel, there can also be an overpressure, in particular a slight overpressure, in particular so that no particles can get into the sterilisation tunnel.

In a further aspect of the present invention, use of the particle counting device as has already been described or will be described below is proposed for particle counting in a sterilisation tunnel of a medicament filling system.

In a further aspect of the present invention, a method for particle counting in a sterilisation tunnel of a medicament filling system using the particle counting device is proposed, as has already been described or will be described below. The particle counting method can also be referred to as a leak test.

A fundamental distinction is drawn between two different operating states of the sterilisation tunnel. The first operating state can be described as “at rest”. When “at rest,” the entire sterilisation tunnel can be in a cold state while performing the particle counting method. In particular, the fans can be in operation to supply air to the sterilisation tunnel. However, the heater can be switched off and in particular, there can be no vessels on the conveyor belt. A second operating state can be described as “in operation”. When “in operation”, a clean room class determination can be carried out under production conditions. The sterilisation tunnel can be in a hot state. The fans for supplying air to the sterilisation tunnel can be running, the heater can be switched on and there can also be no vessels on the conveyor belt.

The particle counting method is carried out strictly in the “at rest” state of the system. The particulate filter, in particular the HEPA filter, can be acted upon at a nominal volume flow on an untreated air side. A nominal volume flow basically means a quantity of air at which the particulate filter can be used in the sterilisation tunnel under operating conditions. Air in the supply air duct in front of the particulate filter can be defined as untreated air. Air after flowing through a particulate filter can be defined as clean air.

When carrying out the particle counting method, an aerosol generator can generate a constant test aerosol with defined properties. A particle concentration can be adjusted using an adjustable flow meter with a needle valve. Di-2-ethylhexyl-sebacate (DEHS) can be used as the particle material. In front of the particulate filter, on the untreated air side, this test aerosol can be introduced via a test socket. The particle concentration on the untreated air side can be monitored by a particle counter with a dilution stage connected upstream of it. The dilution stage can reduce the sucked-in particles with a dilution factor of 1:1000. The particle counter can suck in the air with the particles, measure their size and quantity and evaluate them. A sensor of the particle counter can measure and count particles with a size of 0.3 μm to 10 μm. The dilution stage may be necessary because the particle counter can measure strictly a maximum concentration of the particles and this concentration may be exceeded without a dilution stage.

On the clean air side, the number of particles can be measured with a defined isokinetic probe, the tube of which is connected to another particle counter with a suitable hose. During measurement, the entire filter surface can be moved in steps, as is explained in more detail below. The further particle counter can suck in the emerging clean air via the probe and evaluate a measured number of particles.

As part of the particle counting method, the defined number of particles on the clean air side per measurement process can depend on the number of particles on the untreated air side. The exact ratios of the particle count on the untreated air side and the permitted particle count on the clean air side can be described and determined within the company. By checking the function of the particulate filter, it can be ensured that even with a defined increased number of particles on the untreated air side, the clean room zone within the sterilisation tunnel is not contaminated, in particular not contaminated at certain points. As part of the particle counting method, the scanner can carry out the steps listed below at the same time as the start of particle counting, in particular independently scanning an area under the particulate filter.

The method includes the steps listed below. The method may include further steps not mentioned.

The particle counting method includes the following steps:
a) a movement, in particular a stepwise movement, of the transverse runner in the transport direction of the conveyor belt by means of the bogie; and
b) guiding the probe holder transversely, in particular essentially perpendicularly, to the transport direction by means of the linear guide, preferably at a constant speed.

Steps a) and b) are carried out one after the other and repeatedly.

During step a), the bogie can be moved a defined distance in the transport direction of the conveyor belt. The distance can be called an increment. Before carrying out step a), the probe holder can be arranged at a first end of the linear guide. During step b), the probe holder can be guided from the first end to a second end of the linear guide. This can result in a meandering track of the probe holder. Furthermore, the probe can comprise a probe opening, in particular a probe funnel, with an outer diameter, wherein step a) is carried out such that an increment of the particle counting device in the transport direction of the conveyor belt by means of the bogie is smaller than the outer diameter of the probe opening. This means that the area under the particulate filter can be scanned in overlapping paths. Further details can be found in FIG. 16 and the associated description given below it. Steps a) and/or b) can, in particular, be executed manually or automatically. For further details, reference is made to the description above. In particular, the method can include particle counting by means of the particle counter. In particular, particle counting can take place during step b) or during steps a) and b).

The method can, in particular, be a computer-implemented method. The term “computer-implemented” as used herein is a broad term that should be given its ordinary and common meaning as understood by those skilled in the art. The term is not limited to a specific or adapted meaning. The term can, without limitation, refer in particular to a process that is implemented completely or partly using data processing means, in particular using at least one processor. Due to the programmed travel speed and travel path, reproducibility of particle counting can be guaranteed, even on the rearmost filter surfaces.

As stated above, the particle counting device can comprise the at least one temperature sensor. While the method is being carried out, a temperature in the sterilisation tunnel can be recorded using the at least one temperature sensor and step a) can be aborted if the temperature exceeds a defined limit.

As stated above, the linear guide can have at least one drive. The drive can have at least one first motor. Furthermore, the particle counting device can have at least one second motor, which is configured to drive the bogie.

The controller can each have at least one digital output for a motor driver release and at least one digital output for a direction of movement for the first motor and for the second motor. When carrying out step a) and/or step b), the following sequence of steps can be executed:
i. activation of the digital output for motor driver release; and
ii. generation of a pulse signal.

In particular, when carrying out step a), the digital output for motor driver release of the second motor driver of the second motor can be activated. In particular, when carrying out step b), the digital output for motor driver release of the first motor driver of the first motor can be activated.

The steps i. and ii. can be executed at different times from each other. In particular, steps i. and ii. can be executed at a time interval of 100 ms or 200 ms. Other time intervals are certainly also conceivable.

In particular, after carrying out step i. the digital output for the direction of movement is activated. In particular, the digital output for the direction of movement can be activated in step a) when the bogie moves in the transport direction, in particular with a forward movement. When the bogie moves against the transport direction, especially with a backwards movement, the digital output for the direction of movement can be deactivated. In particular, the digital output for the direction of movement in step b) can be activated when the probe holder is guided from the first end of the guide rail to the second end of the guide rail and, when guiding the probe holder from the second end of the guide rail to the first end of the guide rail, the digital output for the direction of movement can be deactivated, or vice versa.

As stated above, the particle counting device, in particular the controller, can comprise the at least one first forward and reverse counter, the at least one second forward and reverse counter and the at least one further forward and reverse counter. First pulses of the first motor can be counted using the at least one first forward and reverse counter, and second pulses of the second motor can be counted using the at least one second forward and reverse counter. A position of the probe on the linear guide can be determined by means of the first pulses of the first motor and a position of the bogie on the conveyor belt can be determined by means of the second pulses of the second motor. After carrying out step a), the further forward and reverse counter of the second motor can be reset to zero. After carrying out step b), the first forward and reverse counter of the first motor can be reset to zero when the probe is at the first end stop. If a particle count exceeds a defined limit, the position of the probe on the conveyor belt can be recorded, in particular stored, as explained in more detail above.

In a further aspect of the present invention, a computer program is proposed. When run on the controller of a particle counting device, as has already been described or will be described below, the computer program carries out the method as has already been described or will be described below.

In a further aspect of the present invention, a computer program product, comprising program code means stored on a machine-readable medium, is proposed for carrying out a method as already described or to be described below, if the program is executed on the controller of a particle counting device as already described or to be described below.

A computer program product is understood to mean the program as a tradable product. It can essentially exist in any form, for example on paper or a computer-readable data carrier, and can in particular be distributed via a data transmission network. In particular, the program code means can be stored on a computer-readable data carrier and/or a computer-readable storage medium. The terms “computer-readable data carrier” and “computer-readable storage medium” as used herein may refer in particular to non-transitory data storage, for example a hardware data storage medium on which computer-executable instructions are stored. The computer-readable data carrier or the computer-readable storage medium can in particular be or include a storage medium such as a random access memory (RAM) and/or a read-only memory (ROM).

In addition, within the scope of the present invention, a data carrier is proposed on which a data structure is stored, which can execute the method, as has already been described or will be described below, after being loaded into a working memory and/or main memory of a computer or computer network.

Finally, within the scope of the present invention, a modulated data signal is proposed which contains instructions that can be executed by a computer system or computer network for executing a method as has already been described or will be described below.

With regard to the computer-implemented aspects of the invention, one, several or even all method steps of the method according to one or more of the embodiments proposed here can be carried out using a computer or computer network. Thus, in general, any of the method steps, including providing and/or manipulating data, may be performed using a computer or computer network. In general, these steps may include any of the method steps except those steps that require manual work, such as providing samples and/or certain aspects of performing actual measurements.

The proposed devices and methods have numerous advantages over known devices and methods.

Particle measurement in the sterilisation tunnels generally ensures that the particulate filters used in them are functioning properly. With the manual guidance of the probe known from the prior art, an inspector can record an entire filter surface of a particulate filter.

However, the speed of the probe movement and the travel path are subject to greater or lesser fluctuations with each test. Due to the current manual guidance of the probe by an inspector, errors can occur, which means that possible leaks are not recorded. When measuring a particle concentration increased at certain points, the inspector also has the difficulty of finding the exact location where the deviation was initially measured. This can in turn involve increased time expenditure.

The proposed devices and methods can be used to achieve partially automated particle measurement. In particular, the scanner can be used to guide the probe semi-automatically, in particular for scanning filter surfaces of different sizes as part of the particle measurement of particulate filters in various sterilisation tunnels. The particle counting device can be used flexibly in sterilisation tunnels with different dimensions.

Partial automation of the leak test for particulate filters can save time and costs. The partial automation of the process basically ensures that the probe for particle measurement can travel over the filter surface, in particular the entire filter surface, at least largely consistently, in an optimal path and at a constant speed. On the one hand, this can lead to high quality of filled medicaments. In addition, rejects can be substantially reduced because any leaks in the particulate filter can be discovered and localised. On the other hand, the inspector can be relieved of strain, as he no longer has to wield long and cumbersome rods, in particular the pipeline.

Process automation can further lead to an improvement in the measurement process, as the measurement process can be carried out in a shorter time and under reproducible conditions. This can also increase the availability of the medicament filling system.

The probe can be guided over a filter surface of the particulate filter to be measured in a defined path and at a defined speed without slipping. Trajectories of the specified path can overlap by a certain value, but do not exceed this value. The semi-automatic guidance of the probe can still be flexibly adapted to the different types of sterilisation tunnel operated in the company. This allows the probe to be guided across the entire area. The travel path can be optimally adjusted in order to be able to reproducibly cover the entire filter surface of the particulate filter in the sterilisation tunnel and, on the other hand, to minimise the testing time and thus increase system availability.

The particle counting device is easy to handle and can be flexibly adapted to different dimensions of the different sterilisation tunnel types. The size and weight of the particle counting device can be kept as low as possible. A total height of the particle counting device can be smaller than a lowest passage height in the sterilisation tunnel, but as low as is structurally feasible. The probe can represent a highest point of the particle counting device. The components can be selected such that programming and visualisation of the travel path is possible. Additionally, operator safety can be respected to avoid injury through proper use.

In order to make handling the particle counting device easier for the inspector, the total weight of the particle counting device can be as low as possible. To reduce weight, light materials such as aluminium or plastic, e.g. polytetrafluoroethylene (PTFE) or polyoxymethylene (POM), can preferably be used. Stainless, austenitic steels can also be used, which are only used to a limited extent due to their higher weight. Since the particle counting device can be used in a production plant in the pharmaceutical sector, materials used can be cleaned with surface disinfectants, which can include, for example, isopropanol. The requirements described in Regulation (EC) No. 1935/2004 can be met. The particle counting method can be carried out at room temperature. Therefore, the heat resistance of the materials is essentially less important. Standard components can at least largely be used in order to be able to procure and replace required parts quickly and easily if necessary. This also allows manufacturing costs to be minimised.

The conveyor belts of the sterilisation tunnels can be made of a wire mesh made of stainless steel. The wire mesh can provide the conveyor belt with necessary air permeability, flexibility and heat resistance in order to function reliably under the prevailing conditions, such as high temperature. However, due to a coarse mesh structure of the conveyor belt, the resulting contact area between the scanner and the wire mesh can be very small. Accordingly, a small amount of friction can arise here. Friction can be increased by attaching O-rings to the wheels, particularly the drive wheels and/or the rear wheels.

Furthermore, the scanner can move in a slip-free manner, so that there are no or only slight deviations in the travel path between two measurements. In addition, abrasive wear on components of the scanner and between the sterilisation tunnel and the scanner is avoided or at least reduced. This can prevent particles from being introduced into the sterilisation tunnel. Components that do not require lubrication with oils or fats can also be used. In this way, contamination in the sterilisation tunnel can be strictly avoided.

The scanner can have at least two modules, the transverse runner and the bogie. The particle counting device can have a maximum height of 160 mm and a maximum width of 600 mm. In particular, the particle counting device can have a height of 116 mm. This can make it easier to handle when the inspector inserts and positions the particle counting device into the sterilisation tunnel before the measurement process.

The transverse runner can be interchangeable. This makes it possible to flexibly adapt and use the particle counting device for different tunnel widths of the sterilisation tunnel. The transverse runner can be mounted on the bogie without tools. A length of the transverse runner can be smaller than a conveyor belt width. A distance between the probe and a side boundary of the conveyor belt can be as small as possible. A travel speed of the probe of 5.9 cm/s when guided transversely to the transport direction of the conveyor belt can be achieved. Slip-free movements can be achieved. The particle counting device can have a very high repeat accuracy.

A total weight of the particle counting device of 5090 g can be achieved. This makes it possible to achieve a compact particle counting device that is easy for the inspector to handle. The transverse runner can be replaced easily by the inspector without additional tools and the particle counting device can thus be adapted to the respective tunnel width of the different sterilisation tunnel types.

With the help of the particle counting device, the particle counting method can be carried out precisely in a predetermined time on a predetermined path and at a predetermined speed. Substantially flawless reproducibility of the measurements is also possible.

In addition, by programming and visualisation of the particle counting device, in particular the scanner, the position of the probe on the conveyor belt can essentially be determined and monitored at any time.

To summarise, the following embodiments are proposed, without limiting further possible configurations:

Ausführungsform 1: Particle counting device for counting particles in a sterilisation tunnel of a medicament filling system, wherein the sterilisation tunnel comprises at least one conveyor belt, wherein the particle counting device comprises:
at least one probe that can be connected to a particle counter for receiving particles in the sterilisation tunnel;
at least one scanner with at least one probe holder for mounting the probe, wherein the scanner comprises:
   at least one transverse runner with at least one linear guide, wherein the linear guide is configured to guide the probe holder transversely, in particular essentially perpendicularly, to a transport direction of the conveyor belt of the sterilisation tunnel;
   at least one bogie, wherein the transverse runner is mounted on the bogie, wherein the bogie is configured to move the linear guide in the transport direction of the conveyor belt; and
   at least one controller, in particular a controller connected to the bogie, wherein the controller is configured to control a movement of the scanner.
      Ausführungsform 2: Particle counting device according to the preceding embodiment, wherein the controller comprises a programmable logic controller.
      Ausführungsform 3: Particle counting device according to one of the preceding embodiments, wherein the probe holder is mounted on the linear guide.
      Ausführungsform 4: Particle counting device according to one of the preceding embodiments, wherein the particle counting device further comprises at least one particle counter that can be connected to the probe.
      Ausführungsform 5: Particle counting device according to the preceding embodiment, wherein the particle counter is designed as a stationary particle counter and wherein the particle counter and the probe are connected to one another by means of at least one pipeline, in particular a flexible pipeline.
      Ausführungsform 6: Particle counting device according to one of the preceding embodiments, further comprising at least one stationary user interface, wherein the user interface is connected to the scanner and wherein a movement of the scanner is controllable by means of the user interface.
      Ausführungsform 7: Particle counting device according to the preceding embodiment, wherein the user interface has a graphical user interface, in particular a graphical user interface with at least one touch screen.
      Ausführungsform 8: Particle counting device according to one of the two preceding embodiments, wherein at least one travel path and/or measuring positions for particle counting are predeterminable by means of the user interface.
      Ausführungsform 9: Particle counting device according to the preceding embodiment, wherein the travel path comprises a meandering pattern with alternating movements of the probe transversely and parallel to the transport direction.
      Ausführungsform 10: Particle counting device according to one of the two preceding embodiments, wherein the at least one travel path and/or the measuring positions are predeterminable in manual operation or in automatic operation.
      Ausführungsform 11: Particle counting device according to the preceding embodiment, wherein furthermore at least one speed can be predetermined for the at least one travel path, in particular in manual operation.
      Ausführungsform 12: Particle counting device according to one of the six preceding embodiments, wherein the user interface is further configured to specifically move the probe to at least one predeterminable probe position and to carry out a particle count there.
      Ausführungsform 13: Particle counting device according to one of the seven preceding embodiments, wherein the user interface is configured to record and in particular display particle counts depending on a probe position, wherein, in particular, the user interface is additionally connected to a particle counter.
      Ausführungsform 14: Particle counting device according to one of the eight preceding embodiments, wherein the user interface is configured to move the linear guide stepwise in the transport direction of the conveyor belt by means of the bogie, wherein the user interface is further configured to move the probe holder transversely, in particular essentially perpendicularly, to the transport direction by means of the transverse runner.
      Ausführungsform 15: Particle counting device according to the preceding embodiment, wherein the probe comprises a probe opening, in particular a probe funnel, wherein the user interface is configured to carry out the stepwise movement of the linear guide in the transport direction of the conveyor belt by means of the bogie in such a way that an increment of the particle counting device in the transport direction of the conveyor belt by means of the bogie is smaller than the outer diameter of the probe opening.
      Ausführungsform 16: Particle counting device according to one of the preceding embodiments, wherein the particle counting device has at least one y position sensor for determining a position of the probe in a dimension in the transport direction on the conveyor belt.
      Ausführungsform 17: Particle counting device according to the preceding embodiment, wherein the y position sensor is connected to the bogie, in particular to a drive of the bogie.
      Ausführungsform 18: Particle counting device according to the preceding embodiment, wherein the bogie has a stepper motor, wherein the y position sensor comprises an incremental encoder of the stepper motor.
      Ausführungsform 19: Particle counting device according to one of the preceding embodiments, wherein the particle counting device has at least one x position sensor for determining a position of the probe in a dimension transverse to the transport direction, in particular essentially perpendicular to the transport direction, on the conveyor belt.
      Ausführungsform 20: Particle counting device according to the preceding embodiment, wherein the x position sensor is connected to the linear guide, in particular to a drive of the linear guide.
      Ausführungsform 21: Particle counting device according to the preceding embodiment, wherein the linear guide has a stepper motor, wherein the x position sensor comprises an incremental encoder of the stepper motor.
      Ausführungsform 22: Particle counting device according to one of the preceding embodiments, wherein the transverse runner comprises at least two limit switches, in particular at least two roller limit switches, for determining the positions of the limits of the probe transverse to the transport direction of the conveyor belt.
      Ausführungsform 23: Particle counting device according to one of the preceding embodiments, wherein the bogie has at least one limit switch, in particular at least one spring rod limit switch, for determining at least one position of at least one limit of the particle counting device in the transport direction.
      Ausführungsform 24: Particle counting device according to one of the preceding embodiments, wherein the particle counting device further comprises at least one temperature sensor, wherein the temperature sensor is configured to record a temperature in the sterilisation tunnel.
      Ausführungsform 25: Particle counting device according to the preceding embodiment, wherein the particle counting device is configured to issue a warning when at least one temperature threshold is exceeded.
      Ausführungsform 26: Particle counting device according to the preceding embodiment, wherein the particle counting device is configured to interrupt a movement of the linear guide in the transport direction of the conveyor belt by the bogie when the at least one temperature threshold is exceeded.
      Ausführungsform 27: Particle counting device according to one of the preceding embodiments, wherein the linear guide is mounted on a base plate, wherein the linear guide is mounted on the bogie by means of the base plate.
      Ausführungsform 28: Particle counting device according to the preceding embodiment, wherein the base plate is made of aluminium.
      Ausführungsform 29: Particle counting device according to the preceding embodiment, wherein the base plate is mounted on the bogie by means of at least one connection selected from the group consisting of: at least one screw connection, at least one click connection, at least one tension lever connection.
      Ausführungsform 30: Particle counting device according to one of the preceding embodiments, wherein the linear guide has a slide bearing.
      Ausführungsform 31: Particle counting device according to one of the preceding embodiments, wherein the linear guide has a guide rail, in particular a T-shaped guide rail, and a guide carriage mounted on the guide rail, wherein the probe can be attached to the guide carriage.
      Ausführungsform 32: Particle counting device according to the preceding embodiment, wherein the guide rail is configured as a floating bearing, in particular as a floating bearing in a direction transverse to the transport direction of the conveyor belt.
      Ausführungsform 33: Particle counting device according to one of the preceding embodiments, wherein the linear guide has at least one drive selected from the group consisting of: a spindle drive; a toothed belt drive; a rack and pinion drive, wherein the linear guide has at least one rack and pinion drive, wherein the rack and pinion drive has at least one rack and at least one spur gear.
      Ausführungsform 34: Particle counting device according to the preceding embodiment, wherein the rack has a round cross-section.
      Ausführungsform 35: Particle counting device according to one of the two preceding embodiments, wherein the rack is made of an austenitic stainless steel.
      Ausführungsform 36: Particle counting device according to one of the three preceding embodiments, wherein the rack is arranged centrally above the linear guide.
      Ausführungsform 37: Particle counting device according to one of the four preceding embodiments, wherein the spur gear is made of polyoxymethylene (POM).
      Ausführungsform 38: Particle counting device according to one of the five preceding embodiments, wherein the drive comprises at least a first motor selected from the group consisting of: a servo motor; a stepper motor.
      Ausführungsform 39: Particle counting device according to the preceding embodiment, wherein the drive comprises the rack and pinion drive comprising the rack and the spur gear, wherein the spur gear is clamped on a shaft of the first motor, wherein the first motor is mounted on the guide carriage with a mounting bracket.
      Ausführungsform 40: Particle counting device according to one of the nine preceding embodiments, wherein the probe holder is mounted on the guide carriage.
      Ausführungsform 41: Particle counting device according to one of the preceding embodiments, wherein the probe holder is made of polyoxymethylene (POM).
      Ausführungsform 42: Particle counting device according to one of the preceding embodiments, wherein the probe holder has at least one groove for receiving the probe, wherein the probe holder further has at least one clamping plate, wherein the clamping plate is configured to fix the probe.
      Ausführungsform 43: Particle counting device according to one of the preceding embodiments, wherein the transverse runner is mounted centrally on the bogie.
      Ausführungsform 44: Particle counting device according to one of the preceding embodiments, wherein the bogie is made of at least one austenitic stainless steel.
      Ausführungsform 45: Particle counting device according to one of the preceding embodiments, wherein the bogie has at least two drive wheels.
      Ausführungsform 46: Particle counting device according to the preceding embodiment, wherein the drive wheels are made of polyoxymethylene (POM).
      Ausführungsform 47: Particle counting device according to one of the two preceding embodiments, wherein the drive wheels each have several O-rings which are arranged spaced apart from one another on at least one peripheral surface of the drive wheels.
      Ausführungsform 48: Particle counting device according to the preceding embodiment, wherein the O-rings are each received in grooves in the peripheral surface of the drive wheels.
      Ausführungsform 49: Particle counting device according to one of the four preceding embodiments, wherein the drive wheels are each mounted on an axle.
      Ausführungsform 50: Particle counting device according to one of the five preceding embodiments, wherein the particle counting device comprises at least a second motor selected from the group consisting of: a stepper motor, a servo motor, wherein the second motor is configured to drive at least one of the drive wheels.
      Ausführungsform 51: Sterilisation tunnel of a medicament filling system, wherein the sterilisation tunnel comprises:
at least one conveyor belt, wherein the conveyor belt is configured to guide at least one vessel along a transport direction of the conveyor belt;
at least one particulate filter; and
at least one particle counting device arranged between the particulate filter and the conveyor belt according to one of the preceding embodiments, wherein the probe of the particle counting device has a probe opening, in particular a probe funnel, facing the particulate filter.
   Ausführungsform 52: Sterilisation tunnel according to the preceding embodiment, wherein the sterilisation tunnel further comprises at least one supply air duct, wherein the supply air duct comprises at least one fan to suck in ambient air.
   Ausführungsform 53: Sterilisation tunnel according to one of the two preceding embodiments, wherein the sterilisation tunnel further comprises at least one suction device, wherein the suction device is configured to suck out air from below the conveyor belt.
   Ausführungsform 54: Use of the particle counting device according to one of the preceding embodiments relating to a particle counting device for particle counting in a sterilisation tunnel of a medicament filling system.
   Ausführungsform 55: Method for counting particles in a sterilisation tunnel of a medicament filling system using the particle counting device according to one of the preceding embodiments relating to a particle counting device, wherein the method comprises the following steps:
a) a movement, in particular a stepwise movement, of the linear guide in the transport direction of the conveyor belt by means of the bogie;
b) guiding the probe holder transversely, in particular essentially perpendicularly, to the transport direction by means of the linear guide;
   wherein steps a) and b) are carried out one after the other and repeatedly.
   Ausführungsform 56: Method according to the preceding embodiment, wherein before carrying out step a) the probe holder is arranged at a first end of the linear guide, wherein in step b) the probe holder is guided from the first end to a second end of the linear guide.
   Ausführungsform 57: Method according to one of the two preceding embodiments, wherein a temperature in the sterilisation tunnel is further recorded during the execution of the method by means of at least one temperature sensor, wherein step a) is aborted if the temperature exceeds a defined limit.
   Ausführungsform 58: Method according to one of the three preceding embodiments, wherein steps a) and/or b) are carried out in manual operation.
   Ausführungsform 59: Method according to one of the four preceding embodiments, wherein steps a) and/or b) are carried out automatically.
   Ausführungsform 60: Method according to one of the five preceding embodiments, wherein the linear guide has at least one drive, wherein the drive has at least a first motor, wherein the particle counting device further has at least one second motor, wherein the second motor is configured to drive the bogie, wherein the controller for the first motor and for the second motor each has at least one digital output for a direction of movement and at least one digital output for a motor driver release, wherein the following sequence of steps is carried out when step a) and/or step b) is performed:
i. activation of the digital output for motor driver release; and
ii. generation of a pulse signal.
   Ausführungsform 61: Method according to the preceding embodiment, wherein steps i. and ii. are carried out at different times from each other.
   Ausführungsform 62: Method according to one of the two preceding embodiments, wherein, after carrying out step i., the digital output for the direction of movement is activated.
   Ausführungsform 63: Method according to one of the four preceding embodiments, wherein first pulses of the first motor are counted by means of at least a first forward and reverse counter, wherein second pulses of the second motor are counted by means of at least a second forward and reverse counter, wherein a position of the probe on the linear guide is determined by means of the first pulses of the first motor and a position of the bogie on the conveyor belt is determined by means of the second pulses of the second motor.
   Ausführungsform 64: Method according to the preceding embodiment, wherein the linear guide has a first end stop and a second end stop, wherein after carrying out step b), the first forward and reverse counter of the first motor is reset to zero when the probe is at the first end stop.
   Ausführungsform 65: Method according to one of the ten preceding embodiments, wherein, if a particle count exceeds a defined limit, the position of the probe on the conveyor belt is recorded.
   Ausführungsform 66: Method according to one of the eleven preceding embodiments, wherein the probe comprises a probe opening, in particular a probe funnel, with an outer diameter, wherein step a) is carried out such that an increment of the particle counting device in the transport direction of the conveyor belt by means of the bogie is smaller than that outer diameter of the probe opening.
   Ausführungsform 67: Computer program, wherein the computer program, when run on the controller of a particle counting device according to one of the preceding embodiments relating to a particle counting device, executes a method according to one of the preceding embodiments relating to a method.
   Ausführungsform 68: Computer program product with program code means stored on a machine-readable carrier in order to carry out a method according to one of the preceding embodiments relating to a method, if the program is executed on the controller of a particle counting device according to one of the preceding embodiments relating to a particle counting device.

### BRIEF DESCRIPTION OF THE FIGURES

Further details and features arise from the following description of exemplary embodiments, in particular in connection with the dependent claims. The respective features can be implemented individually or in combination with one another. The invention is not restricted to the exemplary embodiments. The exemplary embodiments are shown schematically in the figures. The same reference numbers in the individual figures designate the same or functionally identical elements or elements that correspond in terms of their functions.

In detail, the figures show the following:

FIG. 1 shows a side view of an embodiment of a sterilisation tunnel of a medicament filling system;

FIG. 2 shows a schematic structure of a leak test in a sterilisation tunnel;

FIGS. 3 to 14 show different views of an embodiment of a particle counting device or parts thereof;

FIG. 15 shows a schematic plan view of a particle counting device in a sterilisation tunnel;

FIG. 16 shows geometric dimensions of a particle counting device in a sterilisation tunnel;

FIGS. 17A to 17C show an exemplary sequence of steps for a particle counting method according to the invention (FIGS. 17A and 17C) and an exemplary time diagram of control signals (FIG. 17B);

FIGS. 18A to 18F show various operator interfaces of a user interface (FIGS. 18A, 18B, 18D, 18E and 18F) and a pulse representation (FIG. 18C); and

FIGS. 19 to 20 show exemplary embodiments of programming of a controller.

### DESCRIPTION OF THE EXEMPLARY EMBODIMENTS

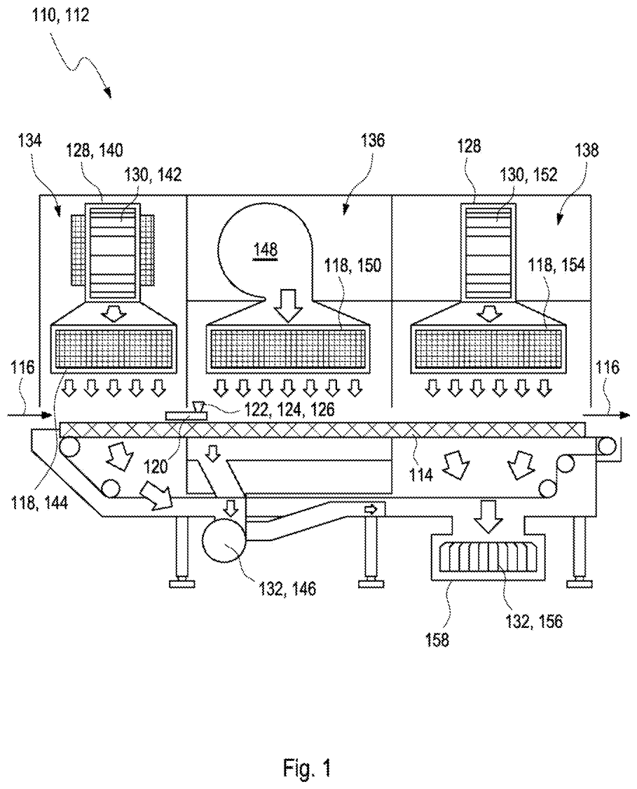

FIG. 1 shows a side view of an embodiment of a sterilisation tunnel 110 of a medicament filling system 112. The sterilisation tunnel 110 includes at least one conveyor belt 114. The conveyor belt 114 is configured to guide at least one vessel (not shown in FIG. 1) along a transport direction 116 of the conveyor belt 114. The transport direction 116 of the conveyor belt 114 is indicated by arrows 116 in FIG. 1.

Furthermore, the sterilisation tunnel 110 includes at least one particulate filter 118 and at least one particle counting device 120 arranged between the particulate filter 118 and the conveyor belt 114, as is described in more detail below. In FIG. 1, the particle counting device 120 is only shown schematically. For a detailed description of the particle counting device 120, reference is made to the description of FIGS. 3 to 14. As shown in FIG. 1, the particle counting device 120 can, in particular, be arranged at least partly on the conveyor belt 114. A probe 122 of the particle counting device 120 has a probe opening 124, in particular a probe funnel 126, facing the particulate filter 118.

The sterilisation tunnel 110 can also include at least one supply air duct 128. The supply air duct 128 can include at least one fan 130 to suck in ambient air. The sterilisation tunnel 110 can further include at least one suction device 132. The suction device 132 can be configured to suck out air below the conveyor belt 114.

As shown in FIG. 1, the sterilisation tunnel 110 can in particular comprise at least three zones. A first zone can be a warm-up zone 134. The warm-up zone 134 can also be referred to as the inlet. The second zone may be a sterilisation zone 136. The sterilisation zone 136 can also be referred to as the hot part. A third zone can be a cooling zone 138. The third zone can also be referred to as the cooling zone 138. The conveyor belt 114 can be configured to move, in particular at a constant speed, through the sterilisation tunnel 110, in particular through the one or more zones of the sterilisation tunnel 110.

The warm-up zone 134 can be configured to slowly bring the vessels up to a temperature of the sterilisation zone 136. In particular, the warm-up zone 134 can be configured to heat, in particular to heat slowly, the glass of the vessels, in particular to reduce tensions that arise in the glass and to avoid possible glass breakage. The sterilisation zone 136 can be configured to vaporise water on and in the vessels. Furthermore, the sterilisation zone 136 can be configured to sterilise the vessels, in particular at a temperature of over 300° C., preferably at 330° C. The cooling zone 138 can be configured to cool, in particular to cool slowly, the vessels, in particular to a temperature of less than or equal to 60° C., in particular in order to reduce built-up tensions in the glass in a controlled manner. The medicament filling system 112 can be configured to transport the vessels from the cooling zone 138 into a filling system, which, however, is not shown in FIG. 1.

The warm-up zone 134 can have at least a first supply air duct 140, at least a first fan 142 and a first particulate filter 144. The first fan 142 and the first particulate filter 144 can be arranged in the supply air duct 128, 140. The first particulate filter 144 can also be referred to as a prefilter. The first fan 142 can be configured to suck in ambient air, in particular ambient air from the clean room, through the first particulate filter 144. The first fan 142 can also be configured to supply the sucked-in ambient air to the first particulate filter 144. The supply air duct 128, 140 can be configured to provide the filtered ambient air as a laminar flow. The filtered air can flow through a conveyor belt area of the sterilisation tunnel 110.

The suction device 132 can include at least one further fan 146. The further fan 146 can in particular be arranged below the conveyor belt 114. The further fan 146 can be configured to suck out air, in particular moist air, below the conveyor belt 114.

The sterilisation zone 136 can work in a circulating procedure. The sterilisation zone 136 can have at least one second fan 148 and at least one second particulate filter 150. The second fan 148 can be configured to suck out air from below the conveyor belt 114 and direct it into an area above the conveyor belt 114. The sterilisation zone 136 can also have at least one heating unit (not shown in FIG. 1), which is configured to heat the air. The second fan 148 can also be configured to supply the heated air, in particular hot air, to the second particulate filter 150. The hot air can emerge, in particular in a laminar flow, above the conveyor belt area.

The further fan 146 can be configured to suck out air, in particular air saturated with water vapour, in the sterilisation zone 136, in particular to prevent the air saturated with water vapour from accumulating in the sterilisation zone 136. The sterilisation zone 136 can also have at least a third particulate filter, wherein the third particulate filter is not shown in FIG. 1. If necessary, ambient air can flow in via the third particulate filter.

The cooling zone 138 can have at least a third fan 152 and at least a fourth particulate filter 154. The third fan 152 can be configured to suck in ambient air, in particular cool ambient air, in particular cool clean room air, and to push it through the fourth particulate filter 154. The emerging laminar flow can be configured to cool the vessels.

The sterilisation tunnel 110 may further have at least a fourth fan 156 and at least one drain channel 158. The fourth fan 156 can, in particular, be a fan of the cooling zone 138. The fourth fan 156 can be configured to suck out air, in particular heated air, in particular air in the cooling zone 138, below the conveyor belt 114 and to supply it to the exhaust air duct 158.

The laminar air flow in the individual zones, in particular in the warm-up zone 134, the sterilisation zone 136 and the cooling zone 138, can flow from top to bottom. Any particles present in the sterilisation tunnel 110 are thereby pushed downwards, which definitely ensures that no particles enter the vessels. In the sterilisation tunnel 110, particularly in the entire sterilisation tunnel 110, there can also be an overpressure, in particular a slight overpressure, in particular so that no particles can get into the sterilisation tunnel 110.

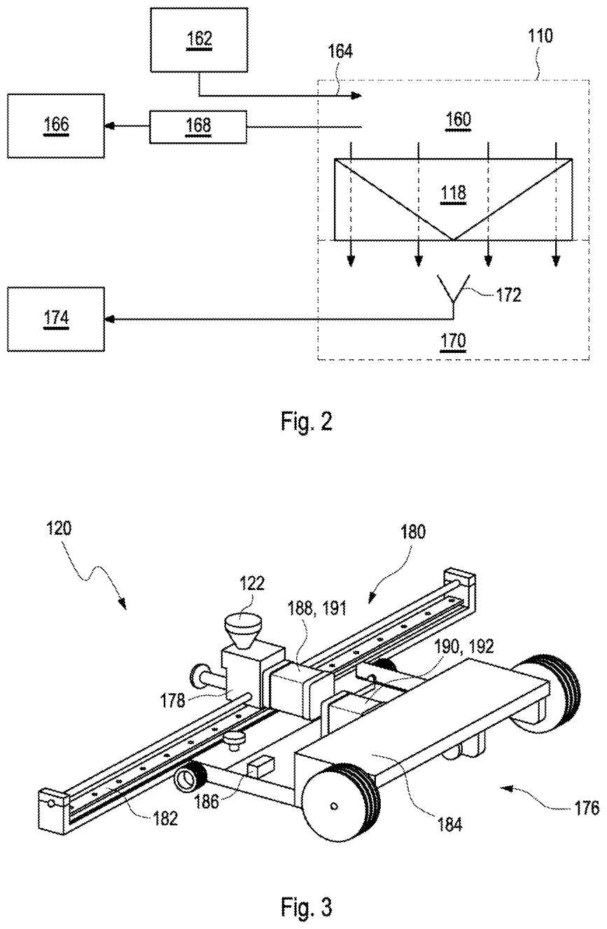

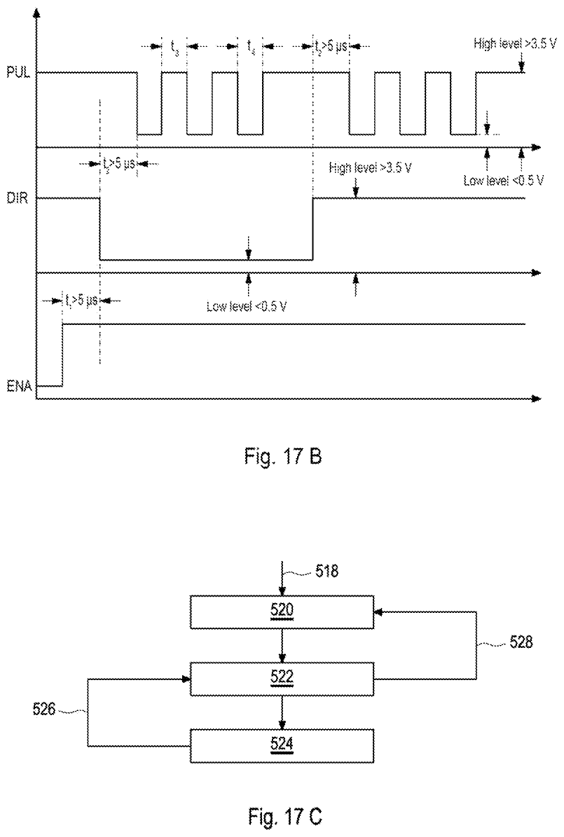

FIG. 2 shows a schematic structure of a leak test in a sterilisation tunnel 110. Setup can be carried out in particular in a method for particle counting in a sterilisation tunnel 110, as is described in more detail below, for example in FIGS. 17A to 17C. The leak test can be carried out in a first and/or a second operating state of the sterilisation tunnel 110. The first operating state can in particular be described as “at rest”. When “at rest,” the entire sterilisation tunnel 110 may be in a cold state while performing the particle counting method. In particular, the fans 130 can be in operation to supply air to the sterilisation tunnel 110. However, a heater can be switched off and in particular there can be no vessels on the conveyor belt 114. The second operating state can be described as “in operation”. When “in operation”, a clean room class determination can be carried out under production conditions. The sterilisation tunnel 110 can be in a hot state. The fans 130 for supplying air to the sterilisation tunnel 110 can be running, the heater can be switched on and there can also be no vessels on the conveyor belt 114.

The particle counting method is carried out strictly in the “at rest” state of the system. The particulate filter 118, in particular the HEPA filter, can be acted upon at a nominal volume flow on an untreated air side 160. A nominal volume flow basically means a quantity of air at which the particulate filter 118 can be used in the sterilisation tunnel 110 under operating conditions. Air in the supply air duct 128 in front of the particulate filter 118 can be defined as untreated air. Air after flowing through a particulate filter 118 can be defined as clean air.

When carrying out the particle counting method, an aerosol generator 162 can generate a constant test aerosol with defined properties. A particle concentration can be adjusted using an adjustable flow meter with a needle valve. Di-2-ethylhexyl-sebacate (DEHS) can be used as the particle material. In front of the particulate filter 118, on the untreated air side 160, this test aerosol can be introduced via a test socket 164. The particle concentration on the untreated air side 160 can be monitored by a particle counter 166 with a dilution stage 168 connected upstream of it. The dilution stage 168 can reduce the sucked-in particles with a dilution factor of 1:1000. The particle counter 166 can suck in the air with the particles, measure their size and quantity and evaluate them. A sensor of the particle counter 166 can measure and count particles with a size of 0.3 μm to 10 μm. The dilution stage 168 may be necessary because the particle counter 166 can measure strictly a maximum concentration of the particles and this concentration can be exceeded without a dilution stage.

On a clean air side 170, the number of particles can be measured with a defined isokinetic probe 172, which can be, for example, the probe 122 of the particle counting device 120, the tube of which is connected to a further particle counter 174 with a suitable hose. During measurement, the entire filter surface can be moved in steps, as is explained in more detail below. The further particle counter 174 can suck in the emerging clean air via the probe and evaluate a measured number of particles.

As part of the particle counting method, the defined number of particles on the clean air side per measurement process can depend on the number of particles on the untreated air side 160. The exact ratios of the particle count on the untreated air side 160 and the permitted particle count on the clean air side 170 can be described and determined within the company. By checking the function of the particulate filter 118, it can be ensured that even with a defined increased number of particles on the untreated air side 160, the clean room zone within the sterilisation tunnel 110 is not contaminated, in particular not contaminated at certain points.

FIGS. 3 to 14 show different views of an embodiment of the particle counting device 120 or parts thereof. FIG. 3 shows a perspective view of the particle counting device 120. The particle counting device 120 for counting particles in the sterilisation tunnel 110 of a medicament filling system 112, wherein the sterilisation tunnel 110, as exemplified in FIG. 1, comprises the at least one conveyor belt 114, comprises the at least one probe 122 that can be connected to the particle counter 174 for receiving particles in the sterilisation tunnel 110. Furthermore, the particle counting device 120 comprises at least one scanner 176 with at least one probe holder 178 for mounting the probe 122. The scanner 176 includes at least one transverse runner 180 with at least one linear guide 182. The linear guide 182 is configured to guide the probe holder 178 transversely, in particular essentially perpendicularly, to the transport direction 116 of the conveyor belt 114 of the sterilisation tunnel 110. Furthermore, the scanner 176 includes at least one bogie 184. The transverse runner 180 is mounted on the bogie 184. The bogie 184 is configured to move the linear guide 182 in the transport direction 116 of the conveyor belt 114. Furthermore, the scanner 176 includes at least one controller 186, in particular a controller 186 connected to the bogie 184, wherein the controller 186 is configured to control a movement of the scanner 176.

The bogie 184 can be configured to move itself and the transverse runner 180, in particular the transverse runner 180 with the probe 122, in a two-dimensional space. The particle counting device 120 can, in particular, in each case comprise a drive for guiding the probe holder 178 transversely (indicated by reference number 188), in particular essentially perpendicularly, to the transport direction 116 of the conveyor belt 114 of the sterilisation tunnel 110 and for moving the bogie 184 (indicated by reference number 190) along the transport direction 116 of the conveyor belt 114. Both drives 188, 190 can be respectively moved with the aid of a motor, for example with a first motor 191 and a second motor 192, as is explained in more detail below. In particular, the particle counting device 120 can be designed such that the movement of the bogie 184 is independent of any guidance of the probe holder 178.

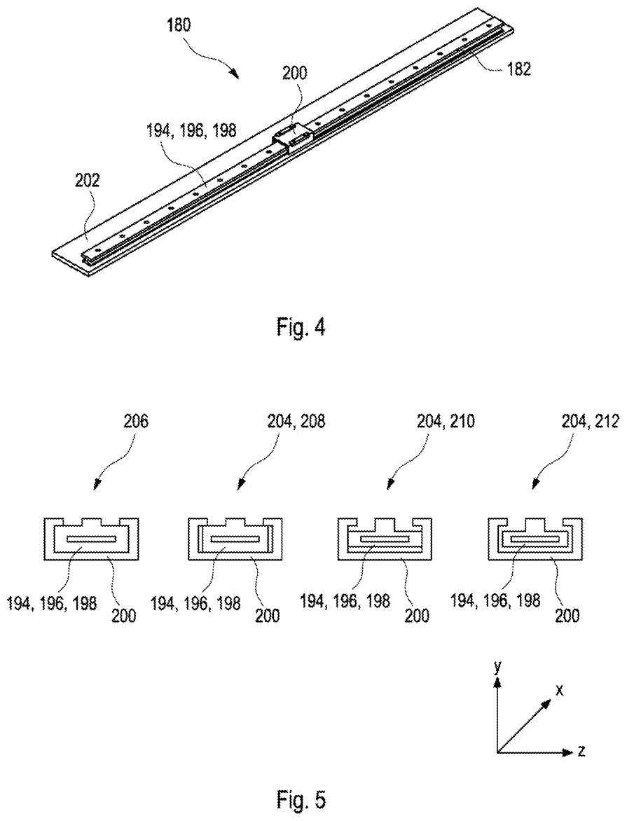

FIG. 4 shows a perspective detailed view of the transverse runner 180. As can be seen in FIG. 4, the linear guide 182 of the transverse runner 180 can comprise at least one guide rail 194, in particular a profiled guide rail 196, in particular a T-shaped guide rail 198. Alternatively and/or additionally, the linear guide 182 can also include a round shaft. Furthermore, the linear guide 182 can comprise at least one guide carriage 200, in particular at least one guide carriage 200 mounted on the guide rail 194 or the round shaft. The probe 122 can be mountable or mounted on the guide carriage 200, in particular by means of the probe holder 178. The linear guide 182 can therefore be configured to guide the probe 122. The linear guide 182, in particular the guide rail 194, can be mounted on a base plate 202, in particular on a base plate 202 made of aluminium, in particular by means of a screw connection. The base plate 202 can be designed to be interchangeable. If the guide rail 194 shows wear, it can be replaced at any time if necessary. The linear guide 182 can be mounted on the bogie 184 using the base plate 202.

The linear guide 182 can in particular have at least one slide bearing, in particular for the guide carriage 200. The slide bearing can be designed to be lubricant-free. However, other bearings such as ball or roller bearings are certainly also conceivable. The T-shaped guide rail 198 makes it entirely possible for the guide carriage 200 with floating bearing 204 to be designed in the direction transverse, in particular perpendicular, to the transport direction 116 of the conveyor belt 114 and/or in the transport direction 116 of the conveyor belt 114. Possible embodiments of the floating bearing 204 are shown in FIG. 5. No floating bearing 206, a floating bearing in the z direction 208, a floating bearing in the y direction 210 and a floating bearing in the yz direction 212 are shown therein.

In this exemplary embodiment, the linear guide 182 comprises the floating bearing in the y direction 210. However, the other floating bearings 204 shown are also possible. The floating bearing 204 allows the guide carriage 200 to have some play in the selected direction. This makes it possible to compensate for manufacturing tolerances in the design. Without floating bearings 204, the system might be rigid, which would mean the guide carriage 200 might tilt, for example. In particular, the guide rail 194 can be configured as a floating bearing 204, in particular as a floating bearing 204 in a direction transverse, in particular perpendicular, to the transport direction 116 of the conveyor belt 114 (indicated by reference number 210). This allows the guide carriage 200 to be able to compensate for small differences in height, in particular in an overall system. A length of the guide carriage 200 can correspond to a flange width of a motor 192.

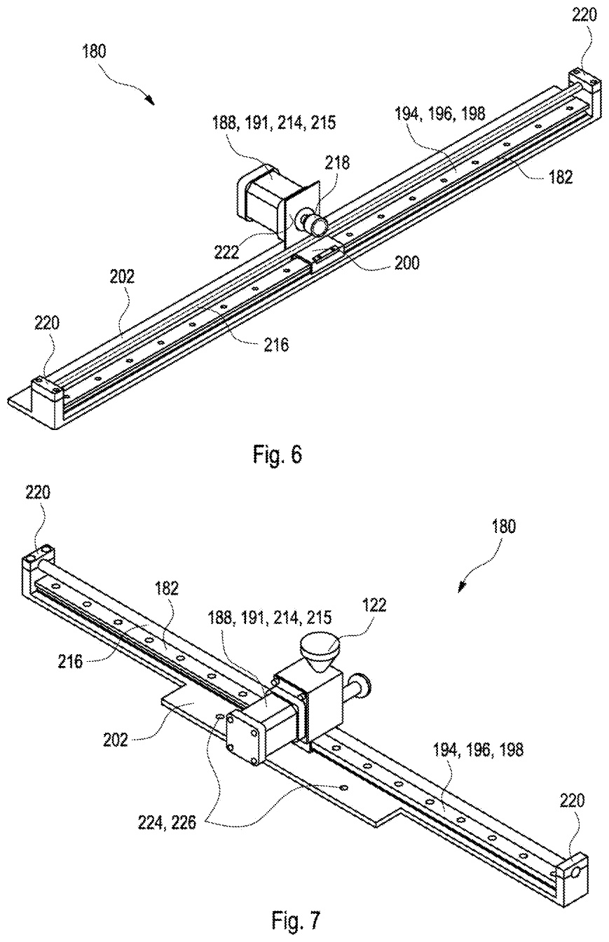

FIG. 6 shows a further perspective detailed view of the transverse runner 180. As stated above, the particle counting device 120 may include a drive 188 for guiding the probe holder 178. For example, the linear guide 182 can have at least one drive 188, in particular a linear drive. The drive 188 can be configured to move the guide carriage 200 on the guide rail 194. The drive 188 can be configured to scan an entire conveyor belt width of the conveyor belt 114 of the sterilisation tunnel 110. As can be seen in FIG. 6, the drive 188 can include a rack and pinion drive 214. However, other embodiments, such as a spindle drive and/or a toothed belt drive, are certainly also conceivable. The drive 188 can, in particular, comprise a stepper motor 215.

The rack and pinion drive 214 can have at least one rack 216 and at least one spur gear 218. The transverse runner 180 can also have at least one, preferably at least two, rack mounts 220. The rack mount 220 can be configured to fix the rack 216, in particular on the base plate 202 of the transverse runner 180. The rack mount 220, can in particular, be made of aluminium. The rack and pinion drive 214 can, in particular, be configured to move the guide carriage 200 over the rack 216, which can in particular be fixed on the base plate 202 of the transverse runner 180, by means of a rotary movement of the spur gear 218. The spur gear 218 can in particular be made of polyoxymethylene (POM). A mounting bracket 222, in particular made of aluminium, can be mounted, in particular screwed, to threaded holes in the guide carriage 200. The stepper motor 215 can also be mounted, in particular screwed, on the mounting bracket 222.

Another perspective detailed view of the transverse runner 180 is shown in FIG. 7. As can be seen in FIG. 7, the base plate 202 can be mounted on the bogie 184 by means of at least one screw connection 224. However, other types of connection, such as a click connection and/or a tension lever connection, are also possible.

The screw connection 224 can, in particular, include knurled screws and/or cylinder head screws. In particular, the base plate 202 can comprise a plurality of boreholes 226, in particular in order to mount the transverse runner 180 on the bogie 184. Due to the high number of transverse runners 180 that need to be changed every year, around six times per year, it can be advantageous to mount the transverse runner 180 on the bogie 184 with knurled screws. This makes it possible to create a compact construction. This means that a tool-free change is certainly possible and the transverse runner 180 can be firmly but releasably connected to the bogie 184. As shown in FIG. 7, the base plate 202 can be adjusted, in particular in order to save weight, by notching out material that is not required. Other embodiments for mounting the transverse runner 180 on the bogie 184 are certainly also conceivable, such as click systems or tension levers.

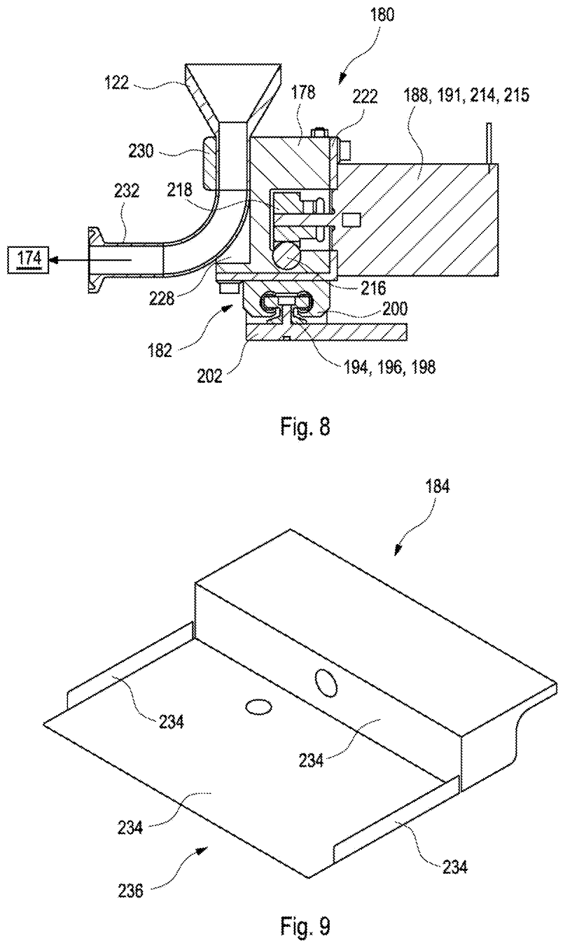

In FIG. 8, the transverse runner 180 is shown in a sectional side view. As can be seen in this view, the probe holder 178 can have at least one groove 228 for receiving the probe 122.

Furthermore, the probe holder 178 can have at least one clamping plate 230, which is configured to fix the probe 122. Alternatively and/or additionally, the probe holder 178 can be mounted on the guide carriage 200.

Furthermore, it can be seen in FIG. 8 that the rack 216 can, in particular, have a round cross-section. The rack 216 can be arranged centrally above the linear guide 182.

As indicated schematically in FIG. 8, the particle counting device 120 can further comprise the at least one particle counter 174 that can be connected to the probe 122. In particular, the particle counter 174 can be designed as a stationary particle counter and the particle counter 174 and the probe 122 can be connected to one another by means of at least one pipeline 232, in particular a flexible pipeline. The pipeline 232 can be part of the particle counter 174 and/or part of the particle counting device 120.

In FIG. 9, the bogie 184 of the particle counting device 120 is shown in a perspective view. The bogie 184 can, in particular, have a frame 234. The frame 234 can also be referred to as the base frame. The frame 234 can, in particular, be made from a metal sheet, in particular from a metal sheet made of austenitic stainless steel. The metal sheet can, in particular, have a thickness of 1 mm to 5 mm, preferably 1.5 mm to 2.5 mm and particularly preferably 2 mm. As stated above, the transverse runner 180 is mounted on the bogie 184. In particular, the transverse runner 180, in particular the base plate 202 of the transverse runner 180, can be mounted on the frame 234. In particular, the transverse runner 180 can be mounted centrally on the bogie 184, in particular on the frame 134. Furthermore, the transverse runner 180 can be mounted to be flush with the bogie, in particular with the frame 234. In particular, the transverse runner 180 can be mounted to be flush with a rear side 236 of the bogie 184, in particular of the frame 234. Due to small run-off surfaces before and after the sterilisation tunnel 110, the size can be minimised and particle counting can be carried out even without run-off zones.

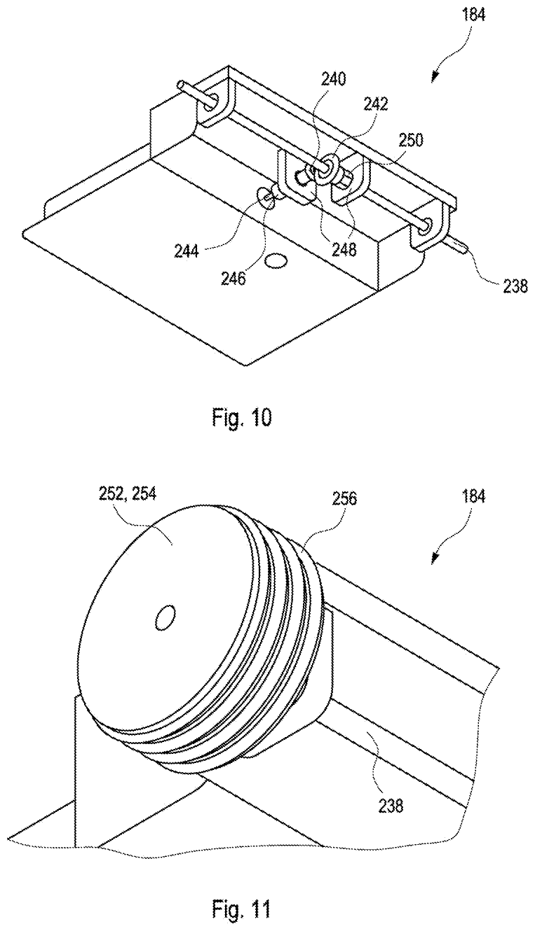

FIG. 10 shows another perspective view of the bogie 184. The second motor 192 of the particle counting device 120 can, for example, comprise at least one stepper motor. However, other types of motors, such as servo motors, for example, are certainly also conceivable. In particular, the second motor 192 can be a NEMA 17 stepper motor (Stepperonline, China) with a related stepper motor driver. Despite its small size, the Nema17 stepper motor can be suitable for generating the power required to drive the movement of the bogie 184. For further details, reference is made to the description above.

The bogie 184 can, in particular, have a drive axle, which is also referred to as an axle 238. The stepper motor can be mounted on the base frame to be offset transversely, in particular at 90°, from the drive axle. The bogie 184 can also have several bevel gears. In order to transmit the rotational movement of the stepper motor to the drive axle, bevel gears with a 2:1 transmission ratio can be selected. A first bevel gear 240 can, for example, have a number of teeth z of 15 and a second bevel gear 242 can have a number of teeth z of 30. The first bevel gear 240 and the second bevel gear 242 can each be made of POM and have a module m of 1.

The first bevel gear 240 can be configured to be driven by the second motor 192 and to transmit a rotational movement to the drive axle via the second bevel gear 242. The transmission ratio allows the speed n₁ of the second motor 192 on the drive axle to be halved and the transmitted torque M₁ to be doubled. As a result, a stepper motor with a small holding torque can generally be used.

A torsionally rigid but angularly and transversely flexible compensating coupling 246 can be used to transmit the torque and speed from the second motor 192 to the shaft 244 of the first bevel gear 240. This can, in principle, compensate for tolerances or misalignments in the design. A compensating coupling 246 used can generally be backlash-free and torsionally rigid and can compensate for both radial and axial angular misalignment. Gear shafts can be held in a suitable position by a holder 248. Grooved ball bearings can be pressed into the holder 248, which support the shaft 244 of the first bevel gear 240 and the axle 238 and ensure smooth running. Grooved ball bearings have the basic advantage that they do not have an increased friction torque when warming up. They also show generally little wear at low speeds and are maintenance-free. The first bevel gear 240 can be friction-locked on the shaft 244 using a set screw. The second bevel gear 242 can also be mounted on the axle 238 with a set screw and can also be secured with an adjusting ring 250 to prevent accidental displacement on the axle 238.

As stated above, the bogie 184 is configured to move the linear guide 182 in the transport direction 116 of the conveyor belt 114. As can be seen in a perspective view in FIG. 11, the bogie 184 can have at least two wheels 252, in particular at least two drive wheels 254, which are configured to move the linear guide 182 in the transport direction 116 of the conveyor belt 114. In this exemplary embodiment, the second motor 192 can be configured to drive at least one of the drive wheels 254, in particular via the axle 238.

In particular, the wheels 252 can be at least partly made of an elastomer. The elastomer can be selected from a group consisting of: silicone, ethylene propylene diene (monomer) rubber (EPDM). However, other elastomers are certainly also conceivable. In particular, the wheels 252 can each have one or more O-rings 256 made of the elastomer. The elastomer can be configured to generate rolling friction between the wheels 252 and the conveyor belt 114. Due to elastic deformability and a high coefficient of friction of the elastomers, a contact area between the O-rings 256 and the conveyor belt 114 can be increased. This means that slip-free operation can be possible.

The wheels 252 can, in particular, be designed in such a way that the O-rings 256 made of the elastomer can be mounted thereon. The wheels 252, in particular the drive wheels 254, can, in particular, be made of polyoxymethylene (POM). In particular, the wheels 252 can have one or more grooves which are configured to receive the O-rings. A diameter of the wheels 252 can be selected such that a low ground clearance is achieved between the frame 234 and the conveyor belt 114. This means that the later overall height of the scanner 176 can be kept to a minimum. The wheels 252, in particular the drive wheels 254, can each have several O-rings 256. In this exemplary embodiment, the wheels 252 preferably have at least four O-rings 256, in particular to increase the contact area between the wheels 252 and the conveyor belt 114. The O-rings 256 can be arranged spaced apart from one another on at least one peripheral surface of the drive wheels 254. The O-rings 256 can each be received in grooves on the peripheral surface of the drive wheels 254.

The drive wheels 254 can each be mounted on the axle 238. A rotational movement of the axle 238 can be transmitted positively to the drive wheels 254 using feather keys. This can prevent the wheels 252 from spinning. In addition, the drive wheels 254 can be secured with a set screw, in particular to prevent them from shifting on the axle 238.

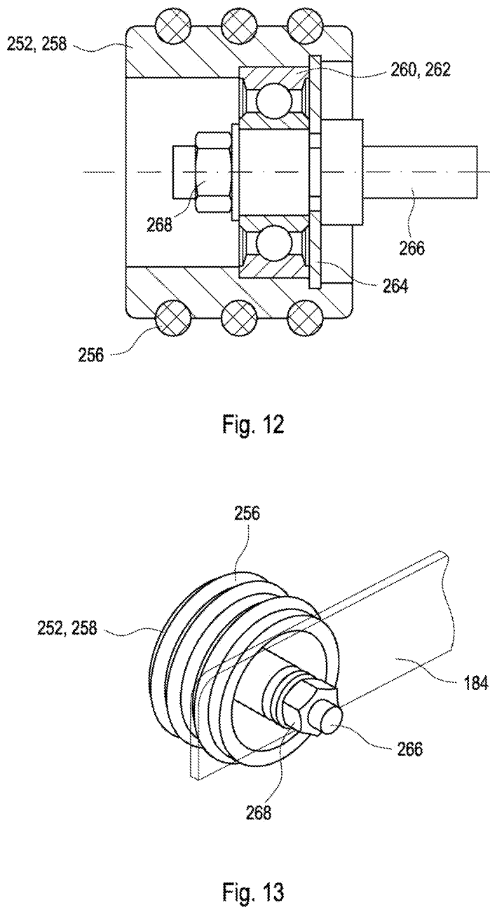

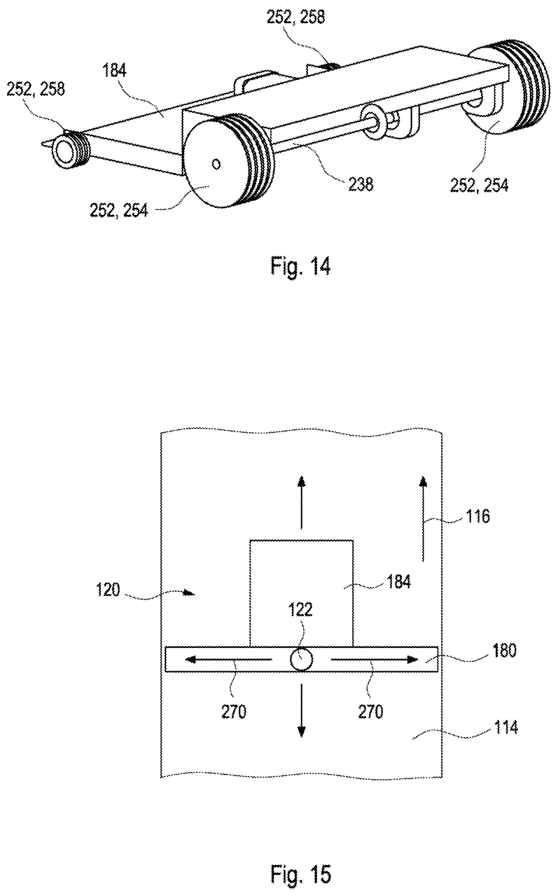

The wheels 252 can further include one or more, in particular two, rear wheels 258. A sectional view of one of the rear wheels 258 is shown in FIG. 12. A perspective view of the rear wheels 258 is shown in FIG. 13. The bogie 184 including the wheels 252, in particular including the drive wheels 254 and the rear wheels 258, can be seen in a perspective view in FIG. 14.

The rear wheels 258 can be designed to be drive-free. The rear wheels 258 can, in particular, run smoothly. For further details of the design of the rear wheels 258, reference is made to the above description, in particular to the description of the drive wheels 254. The rear wheels 258 can be made of POM. Furthermore, the rear wheels 258 can have grooves which can be configured to receive O-rings 256. The O-rings 256 may be made of at least one elastomer. A diameter of the rear wheels 258 can be selected such that the rear wheels 258 do not represent an obstacle to the movement of the transverse runner 180. Ball bearings 260, in particular grooved ball bearings 262, can be pressed into the rear wheels 258, in particular for smooth running. The rear wheels 258 can have a locking ring 264, which is configured to secure the ball bearing 260, in particular the grooved ball bearing 262, against unintentional loosening. The rear wheels 258 can each be pushed onto a shaft 266 and mounted with a self-locking nut 268. The self-locking nut 268 can be configured to prevent the self-locking nut 268 from loosening when the rear wheel 258 rotates. The shafts 266 of the rear wheels 258 can also be mounted, in particular screwed, on the bogie 184 with self-locking nuts 268.

FIG. 15 shows a schematic plan view of the particle counting device 120 in a sterilisation tunnel 110. A fundamental requirement of leak tests is that the filter surface of the particulate filter 118 should be scanned in partially overlapping paths. In order to achieve the required travel path of the probe 122, at least two directions of movement may be necessary. The particle counting device 120 can be configured to move the probe 122 along a conveyor belt width of the conveyor belt 114 (indicated by arrows 270), preferably at a constant speed. In addition, the particle counting device 120 can be configured to carry out a stepwise movement in the transport direction 116 of the conveyor belt 114. These steps can, in particular, be adjusted in such a way that the required overlap is maintained. After particle counting has ended, the particle counting device 120 can return to the starting point.

The drive 190 of the bogie 184 can drive the movement of the entire particle counting device 120 in the transport direction 116 of the conveyor belt 114. The bogie 184 can scan the conveyor belt 114, and thus the particulate filter 118 located above it, along its length and can therefore be used for sterilisation tunnels 110 of any type and size. In order to be able to properly align the particle counting device 120, in particular comprising the bogie 184 and the transverse runner 180, even in the sterilisation tunnel 110 with smaller conveyor belt widths, for example of up to 600 mm, a width of the bogie 184 cannot exceed this width.

The width of the particulate filter 118 can be scanned along the conveyor belt width by moving the probe 122 transversely, in particular orthogonally, to the transport direction 116 of the conveyor belt 114. This can be made possible, in particular, by means of the transverse runner 180. The width of the particulate filter 118 essentially corresponds to the width of the conveyor belt 114 or smaller. The width of the conveyor belt 114 and thus also the dimensions of the particulate filters 118 can vary depending on the type of sterilisation tunnel 110. A length of the transverse runner 180 can therefore be smaller than the width of the conveyor belt 114, since in particular the distance from a side boundary of the conveyor belt 114 to a side wall of the sterilisation tunnel 110 is sometimes only a few millimetres. The different widths of the different sterilisation tunnels 110 can be addressed by an exchangeable transverse runner 180 in order to be able to scan the entire width in each sterilisation tunnel 110. The particle counting device 120 can thus be flexibly adapted to different widths of the particulate filter 118. The transverse runner 180 can be changed without using tools. The components of the transverse runner 180 can, in particular, be selected such that the probe 122 can reach a predetermined travel speed of 5.9 cm/s and can be kept constant. The probe 122 can also be moved as close as possible in terms of construction to the outer boundaries on both sides of the conveyor belt 114 in order to ensure that the largest possible area can be scanned. A maximum passage height in the sterilisation tunnel 110 can also vary. The particle counting device 120 can be suitable for use in all sterilisation tunnels 110, since a maximum overall height of the particle counting device 120 does not exceed the smallest maximum passage height of 160 mm.

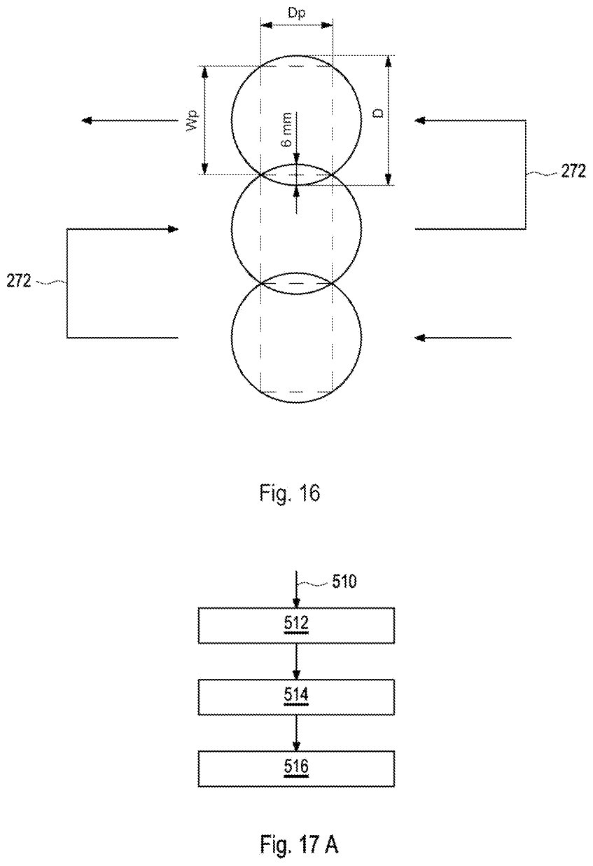

FIG. 16 shows geometric dimensions of the particle counting device 120, in particular a meandering travel path 272 of the particle counting device 120, in a sterilisation tunnel 110.

The filter surface of the particulate filter 118 can thus be scanned with a circular, isokinetic probe 122 with a diameter D of 36 mm in partially overlapping paths. The overlap of the paths is generally set at 6 mm in standard guidelines and should be taken into account as the path distance when moving the probe 122 to the next path. To simplify calculation, a fictitious rectangular probe with edge lengths Wp and Dp is considered. To calculate the number of paths to be scanned per particulate filter 118 and to calculate the measurement duration, the edge lengths Wp and Dp are required. The edge length Wp can correspond to the path distance by which the particle counting device 120 should move forward stepwise. The edge length Dp is the distance from intersection points of the probe paths that overlap by 6 mm.

The edge length Wp can be determined at:

$${W}_{p} = D - 6 mm \qquad ( 30 )$$

$${W}_{p} = 36 mm - 6 mm \qquad ( 31 )$$

$${W}_{p} = 30 mm \qquad ( 32 )$$

The edge length Dp can be determined at:

$${D}_{p} = 2 * \sqrt{{( \frac{D}{2} )}^{2} - {( \frac{D - 6 mm}{2} )}^{2}} \qquad ( 33 )$$

$${D}_{p} = 2 * \sqrt{{( \frac{36 mm}{2} )}^{2} - {( \frac{36 mm - 6 mm}{2} )}^{2}} \qquad ( 34 )$$

$${D}_{p} = 2 * \sqrt{18 {mm}^{2} - 15 {mm}^{2}} \qquad ( 35 )$$

$${D}_{p} = 2 * 9.95 mm \qquad ( 36 )$$

$${D}_{p} = 19.9 mm \qquad ( 37 )$$

Furthermore, a measurement duration t per particulate filter 118, in particular per filter element, can be calculated. A maximum permissible side distance a of the probe 122 from an outer edge of the conveyor belt 114 can be defined respectively as 20 mm on both sides. This value can be maintained by a suitable design of the transverse runner 180. To calculate the measurement duration t per filter element, a filter width B and a filter length L of the filter element can also be used. As stated above, the particulate filter 118 can have a filter length L from 250 mm to 580 mm and a filter width B from 600 mm to 720 mm. A fixed, in particular prescribed, scanning speed v of 59 mm/s results in the following formula:

$$t = \frac{B - 2 * a}{v} * \frac{L}{{W}_{p}} \qquad ( 38 )$$

This results in the measurement time t for a particulate filter 118 with width 700 mm and length 570 mm:

$$t = \frac{700 mm - 2 * 20 mm}{59 \frac{mm}{s}} * \frac{570 mm}{30 mm} \qquad ( 39 )$$

$$t = 212 s \qquad ( 40 )$$

The calculated values for the total measuring time per filter element are listed below; only the total transverse movements of the probe were taken into account: For a particulate filter 118 with a width of 700 mm, this results in t=167 s for a length of 450 mm and t=212 s for a length of 570 mm. For a particulate filter 118 with a width of 600 mm, this results in t=126 s for a length of 400 mm and t=183 s for a length of 580 mm. For a particulate filter 118 with a width of 720 mm, this results in t=100 s for a length of 260 mm, t=153 s for a length of 400 mm and t=215 s for a length of 560 mm. In addition, a duration of the particle counting device 120 for the steps of forward movement in the transport direction 116 of the conveyor belt 114 can be taken into account.

Taking into account a diameter of the drive wheels 254 with O-rings 256, the necessary steps that the stepper motor takes for a movement, in particular a forward movement, of the scanner 176 can essentially be determined. For example, the drive wheel 254 with O-ring 256 can have an outer diameter of 79 mm. The drive wheel can thus have a circumference U of 248.2 mm. With a step angle of 1.8°, the motor, in particular the stepper motor, may need 200 steps for one revolution.

$${n}_{S t e p s} = \frac{200 * {W}_{P}}{U} \qquad ( 41 )$$

$${n}_{S t e p s} = \frac{200 * 30 mm}{248.2 mm} = 24.17 \approx 24 \qquad ( 42 )$$

The stepper motor must therefore move 24 steps in order to move the bogie 184 by a required path distance of 30 mm.

The particle counting device 120, in particular the scanner 176, can be programmed and the user interface configured via the TIA Portal software, in particular in version 13. The programming language SCL (structured control language) can primarily be used here. In individual cases, function blocks can be programmed in FBD (function block diagram, graphic programming language within STEP-7).

A program can include at least one operational part, which in particular includes individual functions required for operation, and at least one documentary part, which contains system messages. This subdivision can increase clarity, which can be particularly helpful when troubleshooting. An operation block OB1, which can be responsible in particular for controlling the scanner 176, can be divided into further function blocks, which are referred to below as FB or FC. The controller of the scanner 176 can be modular. The four possible types of movement, which can be referred to as left, right, forward and backward, can be programmed as separate functions in OB1. Depending on the requirement, for example when guiding the probe holder 178 transversely to the transport direction 116, which can also be referred to as sideways movement, in manual operation or in a propulsion in an automatic measuring mode, these functions can be activated or can remain deactivated. The way the motor driver works can certainly also, for technical reasons, prevent activation of opposite directions of travel. In addition, secondary functions for step counters, conversions and temperature monitoring can be stored in OB1.

The programming of the travel directions can, in particular, be implemented using a sequence of steps.

The controller 186 can in each case have at least one digital output for a motor driver release and at least one digital output for a direction of movement for the first motor 191 and for the second motor 192. The digital output for motor driver release can be referred to as ENA. The digital output for the direction of movement can, in particular, be a digital output for a direction of travel and can be referred to as DIR. In addition, the controller 186 for the first motor 191 and for the second motor 192 can each have at least one digital output for generating a pulse signal, which can also be referred to as PUL.

FIG. 17A shows an exemplary sequence of steps for a particle counting method according to the invention. After controlling a direction of travel, which is shown schematically by arrow 510, the digital output for the motor driver release, ENA, can be activated. This corresponds to field 512 in the sequence of steps. The digital output for the direction of movement, DIR, can then be activated. This corresponds to field 512 in the sequence of steps. This step can be optional. To select an opposite direction of travel, DIR can remain deactivated. A pulse signal can then be generated. The pulse signal may be required for an actual movement. This corresponds to field 514 in the sequence of steps. The sequence of steps can take place with a time lag. For example, the activation of the digital output for the motor driver release, ENA, and the activation of the digital output for the direction of movement, DIR, can occur 100 ms apart. Furthermore, the activation of the digital output for the direction of movement, DIR, and the generation of the pulse signal can take place 100 ms apart.

The programming of the travel directions can be implemented using the sequence of steps shown in FIG. 17A, in particular according to the time diagram of control signals shown in FIG. 17B. Thus, t₁ corresponds to a required time lag between the binary signals for ENA and DIR, t₂ corresponds to a required time lag between the binary signals for DIR and PUL, t₃ corresponds to a duration of how long the binary PUL signal is present and t4 to a duration of how long the binary PUL signal is not present. “High level” corresponds to a voltage greater than 3.5 V DC so that this is definitively identified as a (binary) 1-signal and “low level” corresponds to a voltage of less than 0.5 V DC so that this is definitively identified as a (binary) 0-signal.

The following binary states, which are shown in Tables 1 and 2, thus result for the movement functions.

TABLE 1
Logic table for transverse drive, in particular guidance of the
probe holder 178 transversely to the transport direction 116

| Movement function | ENA | DIR | PUL |
|---|---|---|---|
| Digital output | DQ0.2 | DQ0.3 | DQe0.1 |
| Left | 1 | 1 | 1 |
| Right | 1 | 0 | 1 |

TABLE 2
Logic table of propulsion, in particular movement
of the bogie 184 in the transport direction 116

| Movement function | ENA | DIR | PUL |
|---|---|---|---|
| Digital output | DQ0.0 | DQ0.1 | DQe0.0 |
| Forward | 1 | 1 | 1 |
| Backward | 1 | 0 | 1 |

The resulting travel direction functions can be used to control the movement. In manual mode, it is possible to activate a travel direction selection of a corresponding stored function. In particular, a “left” movement function can be shown with a button that shows an arrow pointing in a left direction. In particular, a “right” movement function can be shown with a button that shows an arrow pointing in a right direction. In particular, a “forward” movement function can be shown with a button that shows an arrow pointing upwards. In particular, a “backward” movement function can be shown with a button that shows an arrow pointing downwards.

In the measuring mode, which can also be referred to as “par-measuring mode”, alternately required travel direction functions can be activated by a programmed loop function for a meandering track, also referred to as a meandering travel path 272.

FIG. 17C shows a further exemplary sequence of steps for a method according to the invention for counting particles in an automatic operation. In particular, after starting a measurement, which is indicated by arrow 518, a movement function can be triggered. In particular, the probe holder 178 can initially have a left end position and a movement function can initially be started to the right, which is shown schematically with field 520. If the probe holder 178 is in the right end position, a movement function can be started in a forward direction, which is shown schematically with field 522. If the probe holder 178 is in the right end position and taking into account a temporary counter, in particular of 30, a movement function to the left can be started, which is shown schematically with field 524. Subsequently, when the probe holder 178 is in the left end position, the movement function can again be started in the forward direction, which is shown schematically with arrow 526. After starting the movement function in the forward direction, with the probe holder 178 in the left end position, and taking into account a temporary counter, in particular of 30, the movement function can be started to the right, which is shown schematically with arrow 528.

Pulses from the motor drivers can be used for the Cartesian position representation and for a distance from measuring paths running transverse to the transport direction 116. In principle, position determination can be carried out without additional use of incremental encoders. Pulses can be counted using a forward and reverse counter for the transport direction 116 and for the direction transverse to the transport direction 116.

To determine a path distance when moving in the transport direction 116, a further, in particular an additional, forward and reverse counter can be used, which, after reaching the next measuring path, is reset to the value zero by the controller 186. In order to prevent increment carryover in the long term, the forward and reverse counter for the direction transverse to the transport direction 116 is zeroed at a left end stop, at which point the forward and reverse counter should have the value zero anyway.

Since the forward and reverse counters are basically configured to count pulses from the motor drivers, the pulses can be converted in a separate function block to display coordinates.

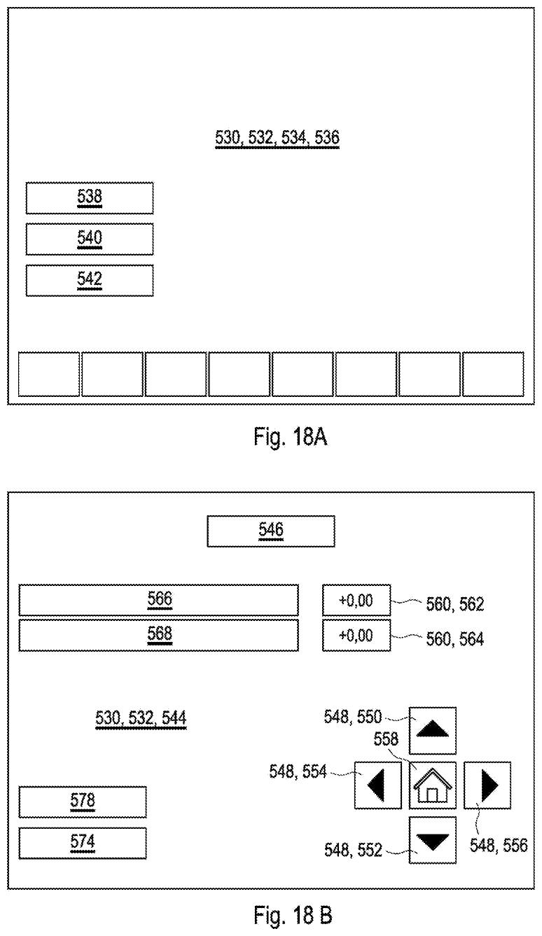

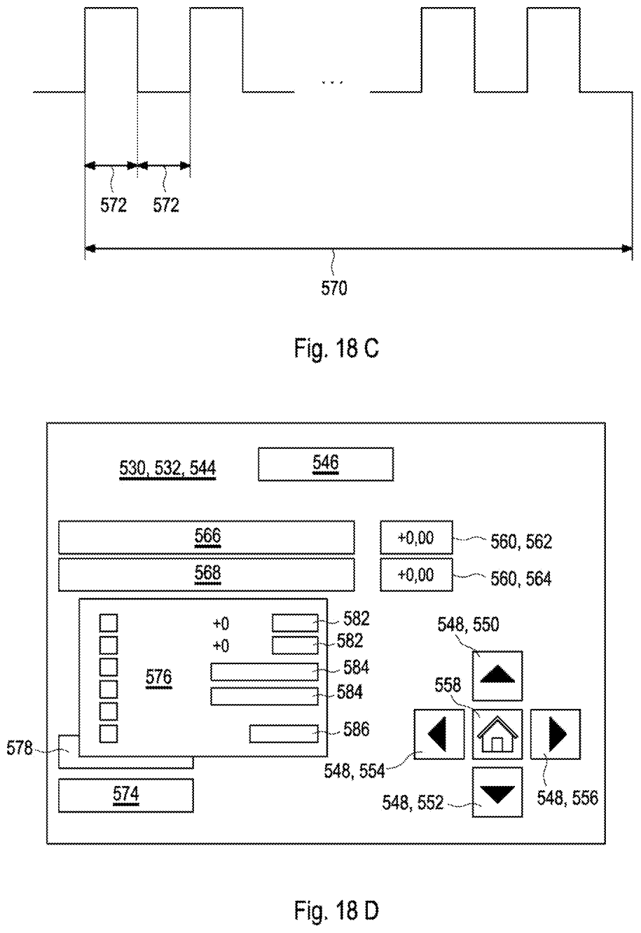

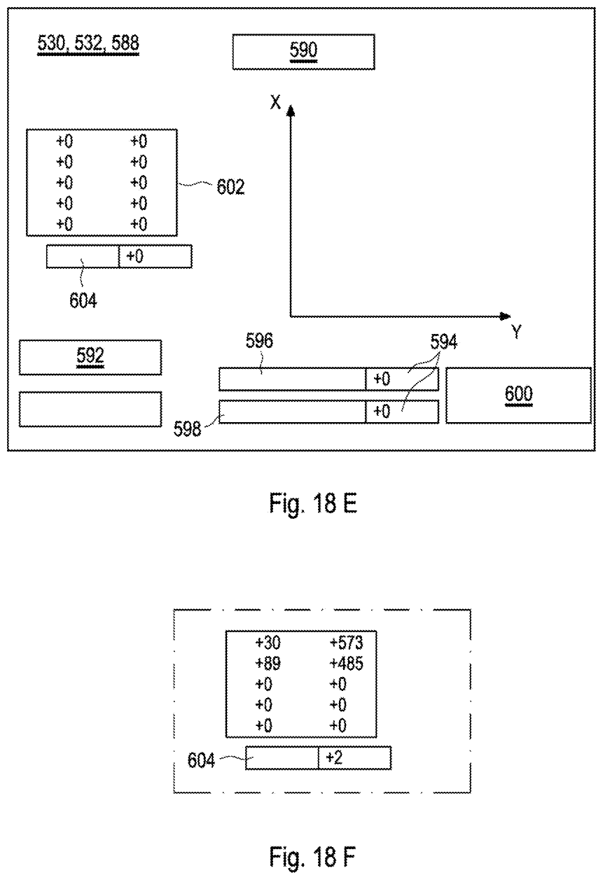

FIGS. 18A to 18F show various operator interfaces of a user interface 530, in particular of a graphical user interface 532 (FIGS. 18A, 18B, 18D, 18E and 18F) and a pulse representation (FIG. 18C).

FIG. 18A shows a main menu 534, which can also be referred to as the basic screen 536. From the main menu 534, three operator interfaces, which can also be referred to as subordinate menu items, can be accessed, in particular through a TIA-internal function, which can be referred to as “ActivateScreen”. In particular, the main menu 534 can have a first button 538, which can have the name “Par-measuring operation” and which can be used to access a first operator interface, which corresponds to an automatic, in particular a partially or fully automatic, operation. Furthermore, the main menu 534 can have a second button 540, which can have the name “Manual operation” and which can be used to access a second operator interface, which corresponds to manual operation, in particular hand operation. Furthermore, the main menu 534 can have a third button 542, which can have the name “Service” and which can be used to access a third operator interface, which corresponds to an interface for system settings.

FIG. 18B shows an exemplary embodiment of a second operator interface 544. The second operator interface 544 can, in particular, have a position 546 at which the label “Manual operation” is displayed. The second operator interface 544 can, in particular, provide a separate control option for directions of movement, in particular directions of movement of the bogie 184 and/or the probe holder 178. The second operator interface 544 can be configured for manual driving of the scanner 176, in particular for manual guiding of the probe holder 178 transversely to the transport direction 116 of the conveyor belt 114 and/or for manual driving of the bogie 184 in the transport direction 116 of the conveyor belt 114. The second operator interface 544 can, in particular, have several buttons 548, which are configured to control the first motor 191 of the linear guide 182 and/or the second motor 192 of the bogie 184, in particular in a direction of travel 116. In particular, the buttons 548 can include at least a first button 550 for controlling a forward movement of the bogie 184 in the transport direction 116. Furthermore, the buttons 548 can include at least a second button 552 for controlling a backwards movement of the bogie 184 against the transport direction 116. Furthermore, the buttons 548 can include at least a third button 554 for controlling the probe holder 178 transversely to the transport direction 116, in particular from the first end of the guide rail 194 to the second end of the guide rail 194. Furthermore, the buttons 548 can include at least a fourth button 556 for controlling the probe holder 178 transversely to the transport direction 116, in particular from the second end of the guide rail 194 to the first end of the guide rail 194. Guiding the probe holder 178 from the first end of the guide rail 194 to the second end of the guide rail 194 can also be referred to as moving or guiding the probe holder 178 to the left. Furthermore, guiding the probe holder 178 from the second end of the guide rail 194 to the first end of the guide rail 194 can also be referred to as moving or guiding the probe holder 178 to the right, or vice versa. The first button 550, the second button 552, the third button 554 and the fourth button 556 can in particular be arranged as a D-pad. Furthermore, the second operator interface 544 can include a home button 558, which can in particular be positioned in the centre of the D-pad.

In particular, the second operator interface 544 can be configured to move the scanner 176 back to a starting position after the particle counting method has been carried out, in particular to move the bogie 184 back to the starting position. In particular, moving the bogie 184 back to the starting position can include a backwards movement of the bogie 184 into the starting position. For this reason in particular, the second operator interface 544 can include at least one, in particular at least two, input fields 560 for a speed. In particular, the second operator interface 455 can include a first input field 562 for a speed of the bogie 184 in the transport direction 116 and a second input field 564 for a speed of the probe holder 178 transverse to the transport direction 116. Before the first input field 560, in particular at a position 566, the second operator interface 544 can comprise the label “Speed in longitudinal direction”. Before the second input field 564, in particular at a position 568, the second operator interface 544 can comprise the label “Speed in transverse direction”. Input speed values can be configured to manipulate holding times of on-switch delays or off-switch delays, which can be used for pulse generators.

As the input fields can be of data type REAL and the holding times of data type TIME, the input value can first be converted or translated to the TIME data type. For the real speed SpeedReal, the following strictly applies:

$${Speed}_{R e a l} = \frac{Path}{Time} \qquad ( 43 )$$

If you now assume one revolution for the speed calculation, the circumference of the driving gear can be obtained for the distance:

$$Path = π * {Diameter}_{g e a r} \qquad ( 44 )$$

Depending on the parameter setting, one revolution can include a defined number of pulses, each of which can be programmed as time delay elements with an identical delay time. This is shown schematically in the pulse representation in FIG. 18C. Arrow 570 shows a complete revolution and arrows 572 show individual time delay elements. Therefore, a product of individual time delays can be used for time in the basic formula for calculating speed:

$$Time = {\sum }_{i = 1}^{{Pul}_{per revolution}} ( 2 * {Pulse}_{Length} ) \qquad ( 45 )$$

This means that a basic formula for calculating speed can be:

$${Speed}_{R e a l} = \frac{π * {Diameter}_{g e a r}}{{Σ}_{i = 1}^{{Pul}_{per revolution}} ( 2 * {Pulse}_{L e n g t h} )} \qquad ( 46 )$$

Because the pulse lengths are always identical, it can be written as follows:

$${Speed}_{R e a l} = \frac{π * {Diameter}_{g e a r}}{2 * {Pulse}_{per revolution} * {Pulse}_{L e n g t h}} \qquad ( 47 )$$

Since the pulse length is required for programming from the entered speed value SpeedReal, the following applies:

$${Pulse}_{Length} = \frac{π * {Diameter}_{g e a r}}{2 * {Pulse}_{per revolution} * {Speed}_{R e a l}} \qquad ( 48 )$$

After the conversion, the data type can be translated, particularly in two steps. Thus, the value can be translated using the following statements:

$${Pulse}_{Length ( D W o r d )} := REAL\_TO \_DWORD ( IN := {Pulse}_{Length ( R e a l )} ) \qquad ( 49 )$$

$${Pulse}_{Length ( T i m e )} := DWORD\_TO \_TIME ( IN := {Pulse}_{Length ( D W o r d )} ) \qquad ( 50 )$$

and can be linked to the corresponding timers.

Furthermore, the second operator interface 544 can have at least one “back” button 574, by means of which the second operator interface 544 switches to the main menu 534 with a screen change and in particular ends the manual mode. Furthermore, the second operator interface 544 can have one or more information windows 576, in particular as one or more test runs, for example for troubleshooting, can be carried out via the second operator interface 544. An exemplary information window is shown in FIG. 18D. The second operator interface 544 can in particular have a service button 578 by means of which the information window 576 is activated. Activation can be realised in the programming in particular by means of a visibility function for screen elements. In particular, the service button 578 can be configured to activate a bit that is used for a visibility query of elements. The information window 576 can in particular have a close button, which can in particular be configured to deactivate the bit, whereby the elements lose their visibility. The information window 576 can have several fields 580, each of which corresponds to the digital outputs of the motor drivers and can each be labelled with “x-ENA”, “x-DIR”, “x-PUL”, “y-ENA”, “y-DIR” and “y-PUL”. The information window 576 can also include one or more buttons 582 for resetting increments for the movement of the bogie 184 and/or for guiding the probe holder 178. In addition, the information window can have one or more buttons 584 for simulating end stops. The information window 576 can be closed using a close button 586.

FIG. 18E shows an exemplary embodiment of a first operator interface 588. The first operator interface 588 can, in particular, have a location 590 at which the label “Par-measuring operation” is displayed. The first operator interface 588 can in particular include starting and stopping a defined, automatic run of the bogie 184 and/or the probe 122. The first operator interface 588 can in particular include a start button 592 which is used to start a programmed sequence of steps. Furthermore, the first operator interface 592 can be configured to make a stop button visible (not shown in FIG. 18E), which appears in particular at a position of the first operator interface 588 that corresponds to a position of the start button 592. Furthermore, the first operator interface 588 can be configured to stop the programmed sequence of steps via the stop button and in particular to deactivate the visibility of the stop button. Furthermore, the first operator interface 588 can have two output fields 594, which display a current Cartesian position of the probe 122. The label “position of x direction” can be shown in a position 596 in front of one of the output fields 594. The label “position of y direction” can be shown in another position 598 in front of one of the output fields 594. Furthermore, the first operator interface 588 can have a “Save position” button 600. The first operator interface 588 can be configured to temporarily store coordinates recorded by pressing the “Save position” button, in particular coordinates recorded at the time of pressing, in particular in a data block. This allows a counter, particularly a leakage counter, to increase a number of possible leaks. A representation of positions of possible leaks 602 can have several, in particular five, variables, each for a position in the transport direction 116 and for a position transverse to the transport direction 116. The positions can also be referred to as x and y positions. Values of these variables can be zero by default, especially if the leakage counter has a value of zero. As soon as the leakage counter is increased to the value of one, current position values will be displayed in a first pair of variables, in particular a first pair of x-y variables. The representation of positions of possible leaks can in particular have a “clear” button 604, which is configured to reset the variables to the value zero, in particular by resetting the leak counter. As already stated, storage of up to 5 leaks can be provided in particular, since this number of abnormalities indicates a critical condition of the filter element being checked. FIG. 18F shows a detailed representation of the positions of possible leaks 602.

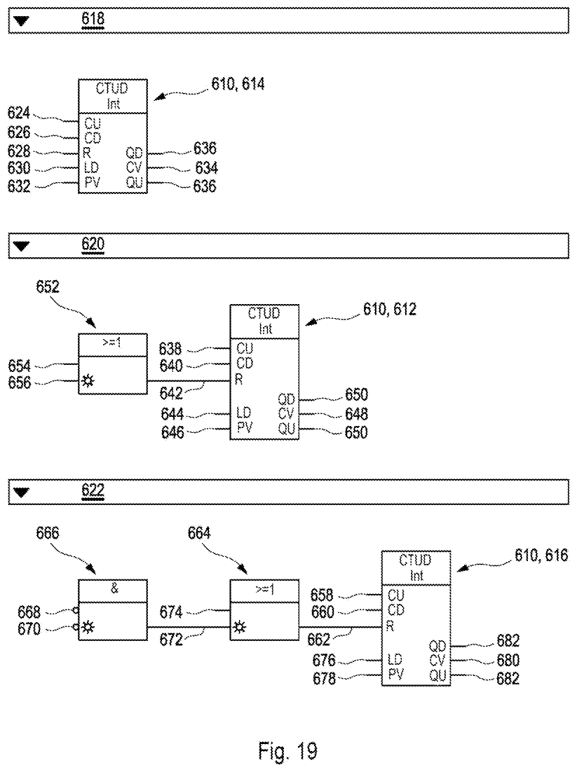

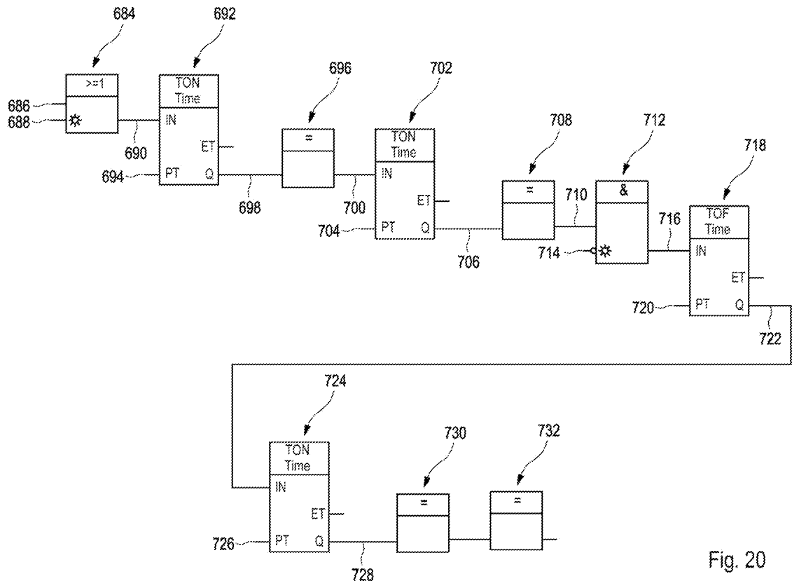

FIGS. 19 and 20 show various exemplary embodiments of programming of the controller 186, in particular of a programmable logic controller (PLC). In these exemplary embodiments, programming is effected via the TIA Portal software in version 13 with the SCL programming language. In particular, FIG. 19 shows an exemplary embodiment of programming of the controller 186 comprising at least one forward and reverse counter 610. The controller 186 may include a first forward and reverse counter 612, a second forward and reverse counter 614, and a third forward and reverse counter 616. As shown in FIG. 19, the second forward and reverse counter 614 can be contained in a first network 618, the first forward and reverse counter 612 in a second network 620, and the third forward and reverse counter 616 in a third network 622 of the controller 186.

The particle counting device 120, in particular the controller 186, can be configured to count second pulses of the second motor 192, in particular of the second stepper motor driver, by means of at least one second forward and reverse counter 614. The second forward and reverse counter 614 can thus, in particular, be a counter on the x axis. A second counting input for counting up 624 can be assigned a “x-PUL_V” signal. A second counting input for counting down 626 can be assigned a “x-PUL_R” signal. A second reset input 628 can be assigned a “Reset_x axis” signal. A second charging input 630 can be assigned a “false” signal. A second load value 632 can be assigned a value of 0. Furthermore, the second forward and reverse counter 614 can have a second output for the counter reading 634 and further second outputs for querying the counter status 636. In particular, the “x position” can be taken from the second output for the counter reading 634.

The particle counting device 120, in particular the controller 186, can be configured to count first pulses of the first motor 191, in particular of the first stepper motor driver, by means of at least a first forward and reverse counter 612. The first forward and reverse counter 614 can thus, in particular, be a counter on the y axis. A first counting input for counting up 638 can be assigned a “y-PUL_R” signal. A first counting input for counting down 640 can be assigned a “y-PUL_L” signal. A first reset input 642 can be assigned a signal from an OR element 652, with which the input signals “Reset y axis” 654 and “Limit_Left” 656 are in contact. A first charging input 644 can be assigned a “false” signal. A first load value 646 can be assigned a value of 0. Furthermore, the first forward and reverse counter 612 can have a first output for the counter reading 648 and further first outputs for querying the counter status 650. In particular, the “y position” can be taken from the first output for the counter reading 648.

The particle counting device 120, in particular the controller 186, can thus be configured to determine a position of the probe 122 on the linear guide 182 using the first pulses of the first motor 191 and a position of the bogie 184 on the conveyor belt 114 using the second pulses of the second motor 192.

The third forward and reverse counter 616 can therefore, in particular, be an “x-axis_temp” counter. A third counting input for counting up 658 can be assigned a “x-PUL_V” signal. A third counting input for counting down 660 can be assigned a “x-PUL_R” signal. A third reset input 662 can be assigned a signal from an OR element 664 with an upstream AND element 666. The input signals 668 “Limit_Left” 670 and “Limit_Right” 670 are in contact with the AND element 666. The OR element 664 is supplied with an output signal 672 of the AND element 666 and an input signal “Reset_x-axis” 674. A third charging input 676 can be assigned a “Right drive” signal. A third load value 678 can be assigned a value of 0. Furthermore, the third forward and reverse counter 616 can have a third output for the counter reading 680 and further third outputs for querying the counter status 682. In particular, the “x-Position_Temp” can be taken from the third output for the counter reading 680.

In addition, the particle counting device 120, in particular the controller 186, can comprise at least one further forward and reverse counter (not shown), which is configured to count pulses of the second motor 192, in particular of the second stepper motor driver. The further forward and reverse counter can therefore be configured in particular to determine a path distance during a movement in the transport direction 116. The particle counting device 120, in particular the controller 186, can be configured to reset the further forward and reverse counter to zero after a stepwise movement of the bogie 184, in particular after reaching a next measuring path. Furthermore, the linear guide 182 can have a first end stop and a second end stop and the controller 186 can be configured to reset the first forward and reverse counter 612 to zero when the probe 122 is at the first end stop. In particular, the first end stop can be a left end stop.

FIG. 20 shows an exemplary embodiment of programming of the controller 186 comprising a sequence of steps for controlling the movement of the particle device 120. The sequence of steps shown in FIG. 20 begins with an OR element 684 with which the input signals “Start_L” 686 or “Initial travel” 688 are in contact. The output signal 690 of the OR element 684 can be in contact as the start condition of a function block 692 with the “Left_ENA” function. A switch-on delay with a time specification 694 of, for example, 100 ms can be present at function block 692. A memory element “y-ENA” 696 can pass on the output signal 698 of the function block 692 as an input signal 700 for a start condition of a further function block 702 with the “Left DIR” function. A switch-on delay with a time specification 704 of, for example, 100 ms can be present at function block 702. An output signal 706 of the function block 702 can be passed on via a memory element “y-DIR” 708 as an input signal 710 of an AND element 712. A further input signal “Pulse_Restart” 714 can be present at the AND element 712. An output signal 716 of the AND element 712 can be in contact as the start condition of a further function block 718 with the “Left PUL” function. There can be a switch-off delay with a time specification 720 “Time_PUL” at function block 718. An output signal 722 of the function block 718 can serve as a starting condition for another function block 724 with the “Waiting_time_for_restart_L” function. A switch-on delay with a time specification “Time_PUL” 726 can be present at function block 724. An output signal 728 of the function block 724 can be present as an input signal from memory elements “Pulse_Restart” 730 and “y-PUL” 732 connected in series.

### EXAMPLES

The following examples serve to explain the invention. They should not be construed as limiting the scope of protection.

The stepper motors were controlled using a board with a microcontroller (Arduino) to check their functionality. The board was connected to a computer via a USB interface. Using the related software, the program for controlling the movement of the linear guide or the movement of the bogie was created and loaded onto the board. The program specified, among other things, the steps, the speed and the direction of rotation of the stepper motor. The board transmitted the signals to the motor driver via a breadboard. The motor driver passed signals to the stepper motor and supplied it with the required voltage. A mains adaptor also connected to the motor driver converted a voltage from 230 V to the 24 V required by the motor driver. The mains adaptor was selected to supply two motor drivers and thus both stepper motors of the particle counting device 120. This setup enabled a realistic functional test to be carried out.

### Example 1: Checking the Repeat Accuracy of the Stepper Motor of the Linear Guide

To check the repeat accuracy of the stepper motor of the linear guide, a movement sequence of the probe holder or the rack and pinion drive was tested in several consecutive paths. This checked whether steps were skipped when the stepper motor rotated. This could essentially lead to a travel path not being completely scanned. This could result in subsequent errors, such as a collision with the rack mount. For the particulate filter 118, in particular the HEPA filter, in a tunnel type with a length L of 570 mm, many paths are basically required to scan the entire filter surface. It should strictly be ensured that a forward movement of the bogie follows each guidance of the probe holder transverse to the transport direction. A number of paths to be scanned no was determined and programmed. A path distance W_{P} of 30 mm was used.

$${n}_{B} = \frac{L}{{W}_{P}} \qquad ( 51 )$$

$${n}_{B} = \frac{570 mm}{30 mm} = 19 \qquad ( 52 )$$

The guide carriage was positioned at an initial distance of 5 mm from the rack mount. A starting position was measured with a digital calliper and the program was started. The guide carriage scanned the number of calculated paths no consecutively at a speed of 5.9 cm/s and then stopped at the end position. In this case, the end position was the same as the start position. The distance between the rack mount and the end position was measured again with the digital calliper.

In order to obtain a meaningful result about average repeat accuracy and to be able to obtain a mean value, this process was repeated five times in succession and the difference between the start and end positions was determined. This is summarised in Table 3 below.

TABLE 3
Measurement results of the repeat accuracy of
the rack and pinion drive of the linear guide

| Run | 1 | 2 | 3 | 4 | 5 |
|---|---|---|---|---|---|
| Starting position | 5.73 mm | 5.71 mm | 5.50 mm | 5.79 mm | 5.70 mm |
| End position | 5.63 mm | 5.86 mm | 5.71 mm | 5.66 mm | 5.75 mm |
| Deviation x | 0.10 mm | 0.15 mm | 0.11 mm | 0.13 mm | 0.05 mm |

An arithmetic mean x₁ of the measured deviations was calculated:

$$\overline{{x}_{1}} = \frac{1}{n} {\sum }_{i = 1}^{n} {x}_{i} \qquad ( 53 )$$

$$\overline{{x}_{1}} = \frac{0.1 mm + 0.15 mm + 0.11 mm + 0.13 mm + 0.05 mm}{5} = 0.1 \; 08 mm \qquad ( 54 )$$

To prove that no steps were skipped, the deviation x₁ must strictly be smaller than the distance travelled by the spur gear per step S₁.

$${S}_{1} = \frac{{l}_{Z}}{\frac{360 °}{Step angle}} \qquad ( 55 )$$

$${S}_{1} = \frac{59.69 mm}{\frac{360 °}{1.8 °}} = 0.299 mm \qquad ( 56 )$$

Thus, l_{z} corresponds to a distance travelled during one revolution of the gear. For further details, reference is made to the descriptions above.

Because the deviation x₁ is smaller than the distance travelled by the spur gear per step S₁, it was possible to demonstrate perfect function and precise repeat accuracy of the stepper motor and thus of the rack and pinion drive of the linear guide. It was shown that the stepper motor does not skip steps. The deviations determined between the start and end positions were smaller than 0.299 mm in all runs and averaged 0.108 mm.

### Example 2: Checking the Repeat Accuracy of the Bogie Stepper Motor

The function of the bogie was then tested. A new program for controlling the bogie was loaded onto the board with a microcontroller, which sequentially moves the bogie forward by a path distance W_{P} of 30 mm at a time and pauses between individual forward movements. This program was used to simulate the movement of the bogie in the sterilisation tunnel. During pauses in the movement of the bogie, the probe holder can be guided on the transverse runner. Here too, the repeat accuracy of the distance travelled was checked.

The repeat accuracy of the bogie was tested on a flat surface. During the test carried out on the flat surface, the drive wheels moved at a speed of 30 revolutions/min. The bogie, with mounted transverse runner, was moved forwards 19 times in steps according to the number of paths n_{b} to be scanned, by a path distance W_{P} of 30 mm each time. The distance travelled by the bogie had to be 570 mm with 19 repetitions. The end position of the bogie was measured from the starting position using a tape measure with 0.50 mm pitch. This process was also repeated five times in succession.

TABLE 4
Measurement results of the repeat accuracy of the bogie drive

| Run | 1 | 2 | 3 | 4 | 5 |
|---|---|---|---|---|---|
| Starting position | 0.0 mm | 0.0 mm | 0.0 mm | 0.0 mm | 0.0 mm |
| End position | 570.0 mm | 570.5 mm | 569.5 mm | 570.0 mm | 569.5 mm |
| Deviation x | 0.0 mm | 0.5 mm | 0.5 mm | 0.0 mm | 0.5 mm |

An arithmetic mean of the deviations x₂ of the travel path of the bogie was determined:

$$\overline{{x}_{2}} = \frac{1}{n} {\sum }_{i = 1}^{n} {x}_{i} \qquad ( 57 )$$

$$\overline{{x}_{2}} = \frac{0. mm + 0.5 mm + 0.5 mm + 0. mm + 0.5 mm}{5} = 0.3 mm \qquad ( 58 )$$

To show that no motor steps were skipped and the bogie moves forward without errors, the deviation x₂ must be substantially smaller than the distance travelled by the drive wheels and thus the bogie per step S₂:

$${S}_{2} = \frac{U}{\frac{360 °}{Step angle}} \qquad ( 59 )$$

$${S}_{2} = \frac{248.2 mm}{\frac{360 °}{1.8 °}} = 1.241 mm \qquad ( 60 )$$

Thus, U corresponds to a circumference of the drive wheels. Reference is made to the above descriptions.

Because the deviation of the travel path x₂ is smaller than the distance travelled by the drive wheels per step S₂, perfect function and correct repeat accuracy of the bogie were demonstrated. It was also shown here that the stepper motor does not skip steps. The deviations determined between the start and end positions were smaller than 1.241 mm in all runs and averaged 0.3 mm.

### Example 3: Parameter Setting of the Motor Drivers

The scanner can be driven by two separate stepper motors with their related motor drivers. For a functioning drive, the current consumption of the connected motors and the number of pulses per revolution can be set on the motor driver.

Type 17HS24-2104S stepper motors can be used, which have a current consumption of up to 2.1 A. The determination of the number of pulses basically depends on the speed as well as on the lowest adjustable time delay for the pulse programming.

If a minimum time delay of 1 ms, a speed of 5 cm/s and the geometry of the driving gear are now used, this results in 596.90 pulses per revolution. Regarding the calculation, reference is made to the above formulas 22 and 23. Since no value can be selected for the delay time that is smaller than 1 ms, the calculated 596.9 pulses per revolution correspond to a maximum in order to be able to guarantee the speed of 5 cm/s.

According to the parameter table of the stepper driver DM556N (see tables 5 and 6), a current consumption of up to 2.3 A and a speed of 400 pulses per revolution can be set.

TABLE 5
Table of parameters for DM556N motor driver, part 1

| | Root-mean | | | |
|---|---|---|---|---|
| Peak current | square current | SW1 | SW2 | SW3 |
| 1.4 A | 1.0 A | ON | ON | ON |
| 2.1 A | 1.5 A | OFF | ON | ON |
| 2.7 A | 1.9 A | ON | OFF | ON |
| 3.2 A | 2.3 A | OFF | OFF | ON |
| 3.8 A | 2.7 A | ON | ON | OFF |
| 4.3 A | 3.0 A | OFF | ON | OFF |
| 4.9 A | 3.5 A | ON | OFF | OFF |
| 5.6 A | 4.0 A | OFF | OFF | OFF |

TABLE 6
Table of parameters for DM556N motor driver, part 2

| Microstep | Pulses per revolution | SW5 | SW6 | SW7 | SW8 |
|---|---|---|---|---|---|
| 2 | 400 | OFF | ON | ON | ON |
| 4 | 800 | ON | OFF | ON | ON |
| 8 | 1600 | OFF | OFF | ON | ON |
| 16 | 3200 | ON | ON | OFF | ON |
| 32 | 6400 | OFF | ON | OFF | ON |
| 64 | 12800 | ON | OFF | OFF | ON |
| 128 | 25600 | OFF | OFF | OFF | ON |
| 5 | 1000 | ON | ON | ON | OFF |
| 19 | 2000 | OFF | ON | ON | OFF |
| 20 | 4000 | ON | OFF | ON | OFF |
| 25 | 5000 | OFF | OFF | ON | OFF |
| 40 | 8000 | ON | ON | OFF | OFF |
| 50 | 10000 | OFF | ON | OFF | OFF |
| 100 | 20000 | ON | OFF | OFF | OFF |
| 125 | 25000 | OFF | OFF | OFF | OFF |

This results in the following parameter setting for the two motor drivers according to Table 7

TABLE 7
Parameter setting of the motor drivers

| | | | | | | | |
|---|---|---|---|---|---|---|---|
| SW1 | SW2 | SW3 | SW4 | SW5 | SW6 | SW7 | SW8 |

OFF
ON

### LIST OF REFERENCE SIGNS

110 Sterilisation tunnel
112 Medicament filling system
114 Conveyor belt
116 Transport direction
118 Particulate filter
120 Particle counting device
122 Probe
124 Probe opening
126 Probe funnel
128 Supply air duct
130 Fan
132 Suction device
134 Warm-up zone
136 Sterilisation zone
138 Cooling zone
140 First supply air duct
142 First fan
144 First particulate filter
146 Further fan
148 Second fan
150 Second particulate filter
152 Third fan
154 Fourth particulate filter
156 Fourth fan
158 Drain channel
160 Untreated air side
162 Aerosol generator
164 Test socket
166 Particle counter
168 Dilution stage
170 Clean air side
172 Isokinetic probe
174 Particle counter
176 Scanner
178 Probe holder
180 Transverse runner
182 Linear guide
184 Bogie
186 Controller
188 Drive for guiding the probe holder
190 Drive for moving the bogie
191 First motor
192 Second motor
194 Guide rail
196 Profiled guide rail
198 T-shaped guide rail
200 Guide carriage
202 Base plate
204 Floating bearing
206 No floating bearing
208 Floating bearing in z direction
210 Floating bearing in y direction
212 Floating bearing in yz direction
214 Rack and pinion drive
215 Stepper motor
216 Rack
218 Spur gear
220 Rack mount
222 Mounting brackets
224 Screw connection
226 Boreholes
228 Groove
230 Clamping plate
232 Pipeline
234 Frame
236 Rear of the bogie
238 Axle
240 First bevel gear
242 Second bevel gear
244 Shaft of the first bevel gear
246 Compensating coupling
248 Holder
250 Adjusting ring
252 Wheel
254 Drive wheel
256 O-ring
258 Rear wheel
260 Ball bearing
262 Grooved ball bearing
264 Locking ring
266 Shaft
268 Self-locking nut
270 Conveyor belt width
272 Meandering travel path
510 Arrow
512 Field
514 Field
516 Field
518 Arrow
520 Field
522 Field
524 Field
526 Arrow
528 Arrow
530 User interface
532 Graphical user interface
534 Main menu
536 Basic screen
538 First button
540 Second button
542 Third button
544 Second operator interface
546 Position
548 Button
550 First button
552 Second button
554 Third button
556 Fourth button
558 Home button
560 Input field
562 First input field
564 Second input field
566 Position
568 Position
570 Arrow
572 Arrow
574 “Back” button
576 Information window
578 Service button
580 Field
582 Button
584 Button
586 Close button
588 First operator interface
590 Position
592 Start button
594 Output field
596 Position
598 Further position
600 Button
602 Representation of positions of possible leaks
604 Clear button
610 Forward and reverse counter
612 First forward and reverse counter
614 Second forward and reverse counter
616 Third forward and reverse counter
618 First network
620 Second network
622 Third network
624 Second counting input for counting up
626 Second counting input for counting down
628 Second reset input
630 Second charging input
632 Second load value
634 Second output to the counter reading
636 Second output for requesting the counter status
638 First counting input for counting up
640 First counting input for counting down
642 First reset input
644 First charging input
646 First load value
648 First output to the counter reading
650 First output for requesting the counter status
652 OR element
654 Input signal
656 Input signal
658 Third counting input for counting up
660 Third counting input for counting down
662 Third reset input
664 OR element
666 AND element
668 Input signal
670 Input signal
672 Output signal
674 Input signal
676 Third charging input
678 Third load value
680 Third output to the counter reading
682 Third output for requesting the counter status
684 OR element
686 Input signal
688 Input signal
690 Output signal
692 Function block
694 Timing
696 Storage element
698 Output signal
700 Input signal
702 Function block
704 Timing
706 Output signal
708 Storage element
710 Input signal
712 AND element
714 Input signal
716 Output signal
718 Function block
720 Timing
722 Output signal
724 Function block
726 Timing
728 Output signal
730 Storage element
732 Storage element

1. A particle counting device for counting particles in a sterilisation tunnel of a medicament filling system, wherein the sterilisation tunnel comprises at least one conveyor belt, wherein the particle counting device comprises:
at least one probe that can be connected to a particle counter for receiving particles in the sterilisation tunnel;
at least one scanner with at least one probe holder for mounting the probe, wherein the scanner comprises:
at least one transverse runner with at least one linear guide, wherein the linear guide is configured to guide the probe holder transversely to a transport direction of the conveyor belt of the sterilisation tunnel;
at least one bogie, wherein the transverse runner is mounted on the bogie, wherein the bogie is configured to move the linear guide in the transport direction of the conveyor belt; and
at least one controller, wherein the controller is configured to control a movement of the scanner.

2. The particle counting device according to claim 1, wherein the controller comprises a programmable logic controller.

3. The particle counting device according to claim 1, wherein the probe holder is mounted on the linear guide.

4. The particle counting device according to claim 1, wherein the particle counting device further comprises at least one particle counter that can be connected to the probe.

5. The particle counting device according to claim 4, wherein the particle counter is designed as a stationary particle counter and wherein the particle counter and the probe are connected to one another by means of at least one pipeline.

6. The particle counting device according to claim 1, further comprising at least one stationary user interface, wherein the user interface is connected to the scanner and wherein a movement of the scanner is controllable by means of the user interface.

7. The particle counting device according to claim 6, wherein the user interface comprises a graphical user interface.

8. The particle counting device according to claim 6, wherein at least one travel path and/or measuring positions for particle counting are predeterminable by means of the user interface.

9. The particle counting device according to claim 8, wherein the travel path comprises a meandering pattern with alternating movements of the probe transversely and parallel to the transport direction.

10. Particle counting device according to claim 8, wherein the at least one travel path and/or the measuring positions are predeterminable in manual operation or in automatic operation.

11. The particle counting device according to claim 10, wherein at least one speed is further predeterminable for the at least one travel path.

12. The particle counting device according to claim 6, wherein the user interface is further configured to specifically move the probe to at least one predeterminable probe position and to carry out a particle count there.

13. The particle counting device according to claim 6, wherein the user interface is configured to record and in particular to display particle counts as a function of a probe position, wherein the user interface is, in particular, additionally connected to a particle counter.

14. The particle counting device according to claim 6, wherein the user interface is configured to move the linear guide stepwise in the transport direction of the conveyor belt by means of the bogie, wherein the user interface is further configured to guide the probe holder transversely to the transport direction by means of the transverse runner.

15. The particle counting device according to claim 1, wherein the probe comprises a probe opening, wherein the user interface is configured to execute the stepwise movement of the linear guide in the transport direction of the conveyor belt by means of the bogie in such a way that an increment of the particle counting device in the transport direction of the conveyor belt by means of the bogie is smaller than the outer diameter of the probe opening.

16. The particle counting device according to claim 1, wherein the particle counting device has at least one y-position sensor for determining a position of the probe in a dimension in the transport direction on the conveyor belt.

17. The particle counting device according to claim 16, wherein the y-position sensor is connected to the bogie.

18. The particle counting device according to claim 17, wherein the bogie has a stepper motor, wherein the y-position sensor comprises an incremental encoder of the stepper motor.

19. The particle counting device according to claim 1, wherein the particle counting device has at least one x-position sensor for determining a position of the probe in a dimension transverse to the transport direction on the conveyor belt.

20. The particle counting device according to claim 19, wherein the x-position sensor is connected to the linear guide.

21. The particle counting device according to claim 20, wherein the linear guide has a stepper motor, wherein the x-position sensor comprises an incremental encoder of the stepper motor.

22. The particle counting device according to one claim 1, wherein the transverse runner comprises at least two limit switches for determining the positions of the limits of the probe transverse to the transport direction of the conveyor belt.

23. The particle counting device according to claim 1, wherein the bogie has at least one limit switch for determining at least one position of at least one limit of the particle counting device in the transport direction.

24. The particle counting device according to claim 1, wherein the particle counting device further comprises at least one temperature sensor, wherein the temperature sensor is configured to record a temperature in the sterilisation tunnel.

25. The particle counting device according to claim 1, wherein the particle counting device is configured to issue a warning when at least one temperature threshold is exceeded.

26. The particle counting device according to claim 1, wherein the particle counting device is configured to interrupt a movement of the linear guide in the transport direction of the conveyor belt by the bogie when the at least one temperature threshold is exceeded.

27. The particle counting device according to claim 1, wherein the linear guide is mounted on a base plate, wherein the linear guide is mounted on the bogie by means of the base plate.

28. The particle counting device according to claim 27, wherein the base plate is made of aluminium.

29. The particle counting device according to claim 27, wherein the base plate is mounted on the bogie by means of at least one connection selected from the group consisting of: at least one screw connection, at least one click connection, at least one tension lever connection.

30. The particle counting device according to claim 1, wherein the linear guide has a slide bearing.

31. The particle counting device according to claim 1, wherein the linear guide has a guide rail and a guide carriage mounted on the guide rail, wherein the probe can be attached to the guide carriage.

32. The particle counting device according to claim 31, wherein the guide rail is configured as a floating bearing.

33. The particle counting device according to claim 1, wherein the linear guide has at least one drive selected from the group consisting of: a spindle drive; a toothed belt drive; a rack and pinion drive, wherein the linear guide has at least one rack and pinion drive, wherein the rack and pinion drive has at least one rack and at least one spur gear.

34. The particle counting device according to claim 33, wherein the rack has a round cross-section.

35. The particle counting device according to claim 33, wherein the rack is made of an austenitic stainless steel.

36. The particle counting device according to claim 33, wherein the rack is arranged centrally above the linear guide.

37. The particle counting device according to claim 33, wherein the spur gear is made of polyoxymethylene (POM).

38. The particle counting device according to claim 33, wherein the drive comprises at least a first motor selected from the group consisting of: a servo motor; a stepper motor.

39. The particle counting device according to claim 33, wherein the drive comprises the rack and pinion drive comprising the rack and the spur gear, wherein the spur gear is tensioned on a shaft of the first motor, wherein the first motor is mounted on the guide carriage with a mounting bracket.

40. The particle counting device according to claim 1, wherein the probe holder is mounted on the guide carriage.

41. The particle counting device according to claim 1, wherein the probe holder is made of polyoxymethylene (POM).

42. The particle counting device according to claim 1, wherein the probe holder has at least one groove for receiving the probe, wherein the probe holder further comprises at least one clamping plate, wherein the clamping plate is configured to fix the probe.

43. The particle counting device according to claim 1, wherein the transverse runner is mounted centrally on the bogie.

44. The particle counting device according to claim 1, wherein the bogie is made of at least one austenitic stainless steel.

45. The particle counting device according to claim 1, wherein the bogie has at least two drive wheels.

46. The particle counting device according to claim 45, wherein the drive wheels are made of polyoxymethylene (POM).

47. The particle counting device according to claim 45, wherein the drive wheels each have several O-rings which are arranged spaced apart from one another on at least one peripheral surface of the drive wheels.

48. The particle counting device according to claim 47, wherein the O-rings are each received in grooves of the peripheral surface of the drive wheels.

49. The particle counting device according to claim 45, wherein the drive wheels are each mounted on an axle.

50. The particle counting device according to claim 1, wherein the particle counting device comprises at least a second motor selected from the group consisting of: a stepper motor, a servo motor, wherein the second motor is configured to drive at least one of the drive wheels.

51. A sterilisation tunnel of a medicament filling system, wherein the sterilisation tunnel comprises:
at least one conveyor belt, wherein the conveyor belt is configured to guide at least one vessel along a transport direction of the conveyor belt;
at least one particulate filter; and
at least one particle counting device arranged between the particulate filter and the conveyor belt according to claim 1, wherein the probe of the particle counting device has a probe opening facing the particulate filter.

52. The sterilisation tunnel according to claim 51, wherein the sterilisation tunnel further comprises at least one supply air duct, wherein the supply air duct comprises at least one fan to suck in ambient air.

53. The sterilisation tunnel according to claim 51, wherein the sterilisation tunnel further comprises at least one suction device, wherein the suction device is configured to suck out air from below the conveyor belt.

54. (canceled)

55. A method for counting particles in a sterilisation tunnel of a medicament filling system by means of the particle counting device according to claim 1 relating to a particle counting device, wherein the method comprises the following steps:
a) a movement of the transverse runner in the transport direction of the conveyor belt by means of the bogie;
b) guiding the probe holder transversely to the transport direction by means of the linear guide;
wherein steps a) and b) are carried out one after the other and repeatedly.

56. The method according to claim 55, wherein before carrying out step a) the probe holder is arranged at a first end of the linear guide, wherein in step b) the probe holder is guided from the first end to a second end of the linear guide.

57. The method according to claim 55, wherein a temperature in the sterilisation tunnel is further recorded during execution of the method by means of at least one temperature sensor, wherein step a) is aborted if the temperature exceeds a defined limit.

58. The method according to claim 55, wherein steps a) and/or b) are carried out in manual operation.

59. The method according to claim 55, wherein steps a) and/or b) are carried out automatically.

60. The method according to claim 55, wherein the linear guide has at least one drive, wherein the drive has at least a first motor, wherein the particle counting device further has at least one second motor, wherein the second motor is configured to drive thebogie, wherein the controller for the first motor and for the second motor each has at least one digital output for a direction of movement and at least one digital output for a motor driver release, wherein the following sequence of steps is carried out when step a) and/or step b) is performed:
i. activation of the digital output for motor driver release; and
ii. generation of a pulse signal.

61. The method according to claim 60, wherein steps i. and ii. are carried out at different times.

62. The method according to claim 60, wherein the digital output for the direction of movement is activated after step i. has been carried out.

63. The method according to claim 60, wherein first pulses of the first motor are counted by means of at least one first forward and reverse counter, wherein second pulses of the second motor are counted by means of at least one second forward and reverse counter, wherein a position of the probe on the linear guide is determined by means of the first pulses of the first motor and a position of the bogie on the conveyor belt is determined by means of the second pulses of the second motor.

64. The method according to claim 63, wherein the linear guide has a first end stop and a second end stop, wherein the first forward and reverse counter of the first motor is reset to zero after step b) has been performed if the probe is at the first end stop.

65. The method according to claim 55, wherein the position of the probe on the conveyor belt is recorded if a particle count exceeds a defined limit.

66. The method according to claim 63, wherein the probe comprises a probe opening with an outer diameter, wherein step a) is carried out such that an increment of the particle counting device in the transport direction of the conveyor belt by means of the bogie is smaller than the outer diameter of the probe opening.

67. (canceled)

68. (canceled)
